(19) United States
(12) Patent Application Publication
(10) Pub. No.: US 2009/0227601 A1
(43) Pub. Date: Sep. 10, 2009

(54) Bradykinin 1 Receptor Antagonists

(75) Inventors: Jiawang Zhu, Thousand Oaks, CA (US); Jian Jeffrey Chen, Camarillo, CA (US); Jason B. Human, Boston, MA (US); Wenyuan Qian, Camarillo, CA (US)

Correspondence Address: AMGEN INC., 1120 VETERANS BOULEVARD, SOUTH SAN FRANCISCO, CA, 94080, US

(73) Assignee: Amgen Inc., Thousand Oaks, CA (US)

(21) Appl. No.: 12/085,872

(22) PCT Filed: Dec. 6, 2006
(86) PCT No.: PCT/US2006/046566

§ 371 Date: Mar. 13, 2009

Related U.S. Application Data
(60) Provisional application No. 60/748,381, filed on Dec. 7, 2005.

(51) Int. Cl. A61K 31/497; C07D 401/06; C07D 401/14; A61P 29/00

(52) U.S. Cl. 514/255.05; 544/408; 544/405

(57) ABSTRACT

The invention encompasses novel compounds and pharmaceutically acceptable derivatives thereof, pharmaceutical compositions and methods for treatment of diseases mediated by B1 bradykinin receptor.

### BACKGROUND OF THE INVENTION

1. Field of the Invention

This invention is directed to compounds that are bradykinin receptor 1 antagonists, compositions comprising the same, and methods for treating diseases mediated by bradykinin 1 receptor such as inflammation-related disorders, including pain.

2. State of the Art

More than two million people in the United States alone are incapacitated by chronic pain on any given day (T. Jessell & D. Kelly, Pain and Analgesia in PRINCIPLES OF NEURAL SCIENCE, third edition (E. Kandel, J. Schwartz, T. Jessell, eds., (1991)). Unfortunately, current treatments for pain are only partially effective, and many cause lifestyle altering, debilitating, and/or dangerous side effects. For example, non-steroidal anti-inflammatory drugs (“NSAIDs”) such as aspirin, ibuprofen, and indomethacin are moderately effective against inflammatory pain but they are also renally toxic, and high doses tend to cause gastrointestinal irritation, ulceration, bleeding, increased cardiovascular risk, and confusion. Patients treated with opioids frequently experience confusion and constipation, and long-term opioid use is associated with tolerance and dependence. Local anesthetics such as lidocaine and mixelitine simultaneously inhibit pain and cause loss of normal sensation. In addition, when used systemically, local anesthetics are associated with adverse cardiovascular effects. Thus, there is currently an unmet need in the treatment of chronic pain.

Pain is a perception based on signals received from the environment and transmitted and interpreted by the nervous system (for review, see M. Millan, Prog. Neurobiol. 57:1-164 (1999)). Noxious stimuli such as heat and touch cause specialized sensory receptors in the skin to send signals to the central nervous system (“CNS”). This process is called nociception, and the peripheral sensory neurons that mediate it are nociceptors. Depending on the strength of the signal from the nociceptor(s) and the abstraction and elaboration of that signal by the CNS, a person may or may not experience a noxious stimulus as painful. When one's perception of pain is properly calibrated to the intensity of the stimulus, pain serves its intended protective function. However, certain types of tissue damage cause a phenomenon, known as hyperalgesia or pronociception, in which relatively innocuous stimuli are perceived as intensely painful because the person's pain thresholds have been lowered. Both inflammation and nerve damage can induce hyperalgesia. Thus, persons afflicted with inflammatory conditions, such as sunburn, osteoarthritis, colitis, carditis, dermatitis, myositis, neuritis, inflammatory bowel disease, collagen vascular diseases (which include rheumatoid arthritis and lupus) and the like, often experience enhanced sensations of pain. Similarly, trauma, surgery, amputation, abscess, causalgia, collagen vascular diseases, demyelinating diseases, trigeminal neuralgia, cancer, chronic alcoholism, stroke, thalamic pain syndrome, diabetes, herpes infections, acquired immune deficiency syndrome (“AIDS”), toxins and chemotherapy cause nerve injuries that result in pain.

As the mechanisms by which nociceptors transduce external signals under normal and hyperalgesic conditions become better understood, processes implicated in hyperalgesia can be targeted to inhibit the lowering of the pain threshold and thereby lessen the amount of pain experienced.

Bradykinin (BK) and the related peptide, kallidin (Lys-BK) mediate the physiological actions of kinins on the cardiovascular and renal systems. However, the active peptides, BK and kallidin, are quickly degraded by peptidases in the plasma and other biological fluids and by those released from a variety of cells, so that the half-life of BK in plasma is reported to be approximately 17 seconds (1). BK and kallidin are rapidly metabolized in the body by carboxypeptidase N, which removes the carboxyterminal arginine residue to generate des-Arg BK or des-Arg kallidin. Des-Arg-kallidin is among the predominant kinins in man and mediates the pathophysiological actions of kinins in man. In addition to being a very potent proinflammatory peptide, des-Arg-BK or des-Arg-kallidin is known to induce vasodilation, vascular permeability, and bronchoconstriction (for review, see Regoli and Barabe, Pharmacological Rev, 32(1), 1-46 (1980)). In addition, des-Arg-BK and des-Arg-kallidin appear to be particularly important mediators of inflammation and inflammatory pain as well as being involved in the maintenance thereof. There is also a considerable body of evidence implicating the overproduction of des-Arg-kallidin in conditions in which pain is a prominent feature such as septic shock, arthritis, angina, and migraine.

The membrane receptors that mediate the pleiotropic actions of kinins are of two distinct classes, designated B1 and B2. Both classes of receptors have been cloned and sequenced from a variety of species, including man (Menke, et al, J. Biol. Chem. 269, 21583-21586 (1994); Hess et al, Biochem. Biophys. Res. Commun. 184, 260-268 (1992)). They are typical G protein coupled receptors having seven putative membrane spanning regions. In various tissues, BK receptors are coupled to every known second messenger. B2 receptors, which have a higher affinity for BK, appear to be the most prevalent form of bradykinin receptor. Essentially all normal physiological responses and many pathophysio-logical responses to bradykinin are mediated by B2 receptors.

B1 receptors, on the other hand, have a higher affinity for des-Arg-BK compared with BK, whereas des-Arg-BK is inactive at B2 receptors. In addition, B1 receptors are not normally expressed in most tissues. Their expression is induced upon injury or tissue damage as well as in certain kinds of chronic inflammation or systemic insult (F. Marceau, et al., Immunopharmacology, 30, 1-26 (1995)). Furthermore, responses mediated by B1 receptors are up-regulated from a null level following administration of bacterial lipopolysaccharide (LPS) or inflammatory cytokines in rabbits, rats, and pigs.

The pain-inducing properties of kinins coupled with the inducible expression of B1 receptors make the B1 receptor an interesting target in the development of anti-inflammatory, antinociceptive, antihyperalgesic and analgesic agents that may be directed specifically at injured tissues with minimal actions in normal tissues.

Certain compounds have been described as bradykinin antagonists. WO 03/07958, published 30 Jan. 2003, describes tetrahydroquinoxalines. Dihydroquinoxalinones are described in a JACS communication. Piperazine-2,3,5-triones are described in Tet. Lett., 40, 7557-7560 (1999). European application 641779, published 8 Mar. 1995, describes 3,6-dioxopiperazines as platelet aggregation inhibitors.

Clearly, there is a need for new, safe and effective treatments for inflammation and pain. Such agents are provided in the present invention.

### SUMMARY OF THE INVENTION

In one aspect, this invention is directed to a compound of Formula (I):

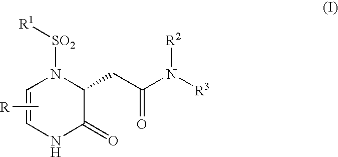

wherein:

R is hydrogen or alkyl;

R¹ is aryl or heteroaryl optionally substituted with R^{a}, R^{b}, and R^{c} independently selected from alkyl, halo, haloalkyl, haloalkoxy, hydroxy, cyano, alkylamino, dialkylamino, or alkoxy;

R² is hydrogen, alkyl, haloalkyl, cycloalkyl, cycloalkylalkyl, hydroxyalkyl, alkoxyalkyl, or aminoalkyl;

R³ is bridged heterocyclyl, bridged cycloalkyl wherein bridged heterocyclyl or bridged cycloalkyl is optionally substituted with one, two or three substitutents selected from alkyl, alkoxy, hydroxyl, hydroxyalkyl, or alkoxyalkyl; or R³ is a group of formula (a):

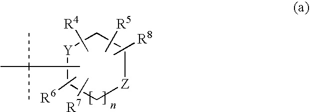

where:

n is 0, 1, 2, or 3;

Y is —CH₂— or NH—;

Z is —(O)—, —C(═NOH), —CONH—, —O—, —C(O)O—, —S—, —SO—, —SO₂—, —NH—, or —CH₂—;

R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano; and

R⁷ and R⁸ are independently hydrogen or alkyl; or

R⁴ and R⁷, when attached to the same carbon atom, can combine together with the carbon atom to which they are attached to form a saturated or unsaturated monocyclic (C₃-C₈)cycloalkyl optionally substituted with R^{d}, R^{e}, and R^{f} as defined above; or a saturated or unsaturated monocyclic heterocyclyl ring containing three to six ring atom wherein one or two ring atoms are selected from —C(O)—, —C(═NOH), —, —S—, —SO—, —SO₂—, or —NH— and wherein the heterocyclyl ring is substituted with R^{m}, Rⁿ and R^{o} where R^{m} and Rⁿ are independently selected from hydrogen, alkyl, hydroxyl, alkoxy, halo, haloalkyl, haloalkoxy, carboxy, or alkoxycarbonyl and R^{o} is selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy and where the aryl, heteroaryl, or heterocyclyl ring in R^{o} is optionally substituted with R^{d}, R^{e}, and R^{f} as defined above; provided that when Y and Z are —CH₂— and R⁴ and R⁷ do not form a cycloalkyl or heterocyclyl ring, then at least one of R⁴, R⁵, R⁶, R⁷, and R⁸ is not hydrogen; or

R² and R³ together with the nitrogen atom to which they are attached form monocyclic heterocyclyl or spiro heterocycloamino each of which is substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R^{g}, R^{h}, and Rⁱ is optionally substituted with R^{j}, R^{k}, or R^{l} independently selected from alkyl, halo, alkoxy, haloalkyl, hydroxyl, carboxy, alkoxycarbonyl, haloalkoxy, or cyano provided that when R² and R³ together with the nitrogen atom to which they are attached form monocyclic heterocyclyl, then at least one of R^{g}, R^{h}, and Rⁱ is not hydrogen;

or an N-oxide; and/or a pharmaceutically acceptable salt thereof provided that: (i) when R is hydrogen, R¹ is 3,4-dichlorophenyl then R² and R³ together with the nitrogen atom to which they are attached do not form 2- and 3-phenylpyrrolidin-1-yl, 2- and 3-benzylpyrrolidin-1-yl, 2-phenylpiperidin-1-yl, 4-phenylpiperidin-1-yl, 4-phenylpiperazin-1-yl, 2-, 3- and 4-hydroxymethylpiperidin-1-yl, 2-, 3-, and 4-benzylpiperidin-1-yl, 2-, 3- and 4-methylpiperidin-1-yl, 3-phenylpiperidin-1-yl, azabicyclo[3.2.2]non-3-yl, azabicyclo[3.2.1]oct-6-yl, 1,3,3-trimethylazabicyclo[3.2.1]oct-6-yl, 2-phenylazepin-1-yl, 4-(pyridin-2-yl)piperazin-1-yl, 3-, or 4-(cyanomethyl)piperazin-1-yl, 4-hydroxyazepan-1-yl, or 4-hydroxy-4-methylazepan-1-yl (ii) when R is hydrogen, R¹ is 4-methylphenyl then R² and R³ together with the nitrogen atom to which they are attached do not form 4-(2-hydroxyethyl)piperazin-1-yl, 2-(4-methoxycarbonylphenyl)pyrrolidin-1-yl, 2-(4-methoxycarbonylphenyl)piperidin-1-yl, 2-(4-hydroxymethylphenyl)piperidin-1-yl, 4-(4-methylpiperazin-1-yl)piperidin-1-yl, 4-(4-phenylpiperidin-1-yl)piperidin-1-yl, 2-(4-piperidin-1-ylmethylphenyl)piperidin-1-yl, or 5-oxa-2-aza-bicyclo[2.2.1]heptan-2-yl (iii) when R is hydrogen, R¹ is 2,3-dichlorophenyl, then R² and R³ together with the nitrogen atom to which they are attached do not form 4-benzylpiperazin-1-yl, 2-phenylpyrrolidin-1-yl, 2-(4-piperidin-1-ylmethylphenyl)piperidin-1-yl, 2-(3-piperidin-1-ylmethylphenyl)piperidin-1-yl, 2-(4-piperidin-1-ylmethylphenyl)pyrrolidin-1-yl, 2-(3-piperidin-1-ylmethylphenyl)pyrrolidin-1-yl, or 2-phenylpiperidin-1-yl (iv) when R is hydrogen, R¹ is 4-methylphenyl, and R² is hydrogen then R³ is not 1-benzylpiperidin-4-yl, 2-3-, and 4-methylcyclohexyl, 2- 3-, and 4-hydroxycyclohexyl, 2-benzyloxycyclohexyl, 2-ethyloxycarbonylcyclohexyl, tricyclo[3.3.1.1˜3,7]dec-1-yl, 3-hydroxytricyclo[3.3.1.1˜3,7]dec-1-yl, admant-1-yl, or 2- or 3-hydroxymethylcyclohexyl, and (v) when R is hydrogen, R¹ is 2,5-dimethylphenyl, and R² is hydrogen then R³ is not 2-methylcyclohexyl.

In a second aspect, this invention is directed to a compound of Formula (Ia):

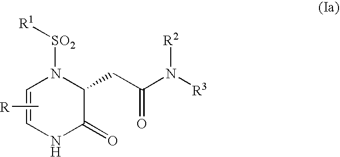

wherein:

R is hydrogen or alkyl;

R¹ is aryl or heteroaryl optionally substituted with R^{a}, R^{b}, and R^{c} independently selected from hydrogen, alkyl, or halo;

R² is hydrogen, alkyl, cycloalkyl, cycloalkylalkyl, hydroxyalkyl, alkoxyalkyl, or aminoalkyl;

R³ is bridged heterocyclyl, bridged cycloalkyl wherein bridged heterocyclyl, or bridged cycloalkyl is optionally substituted with one, two or three substitutents independently selected from alkyl, alkoxy, hydroxyl, hydroxyalkyl, or alkoxyalkyl; or R³ is a group of formula (a):

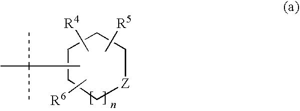

where:

n is 0, 1, 2, or 3;

Z is —C(O)—, —C(═NOH), —CONH—, —, —C(O)O—, —S—, —SO—, —SO₂—, —NH—, or —CH₂—; and

R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano; provided that when Z is —CH₂— then at least one of R⁴, R⁵, and R⁶ is not hydrogen; or

R² and R³ together with the nitrogen atom to which they are attached form monocyclic heterocyclyl substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R^{g}, R^{h}, and Rⁱ is optionally substituted with R^{j}, R^{k}, and R^{l} independently selected from alkyl, halo, alkoxy, haloalkyl, hydroxyl, carboxy, alkoxycarbonyl, haloalkoxy, or cyano provided that at least one of R^{g}, R^{h}, and Rⁱ is not hydrogen;

or a pharmaceutically acceptable salt thereof provided (i) when R is hydrogen, R¹ is 3,4-dichlorophenyl then R² and R³ together with the nitrogen atom to which they are attached do not form 2- and 3-phenylpyrrolidin-1-yl, 2- and 3-benzylpyrrolidin-1-yl, 2-phenylpiperidin-1-yl, 4-phenylpiperidin-1-yl, 4-phenylpiperazin-1-yl, 2-, 3- and 4-hydroxymethylpiperidin-1-yl, 2-, 3-, and 4-benzylpiperidin-1-yl, 2- 3- and 4-methylpiperidin-1-yl, 3-phenylpiperidin-1-yl, azabicyclo[3.2.2]non-3-yl, azabicyclo[3.2.1]oct-6-yl, 1,3,3-trimethylazabicyclo[3.2.1]oct-6-yl, 2-phenylazepin-1-yl, 4-(pyridin-2-yl)piperazin-1-yl, 3-, or 4-(cyanomethyl)piperazin-1-yl, 4-hydroxyazepan-1-yl, or 4-hydroxy-4-methylazepan-1-yl (ii) when R is hydrogen, R¹ is 4-methylphenyl then R² and R³ together with the nitrogen atom to which they are attached do not form 4-(2-hydroxyethyl)piperazin-1-yl, 2-(4-methoxycarbonylphenyl)pyrrolidin-1-yl, 2-(4-methoxycarbonylphenyl)piperidin-1-yl, 2-(4-hydroxymethylphenyl)piperidin-1-yl, 4-(4-methylpiperazin-1-yl)piperidin-1-yl, 4-(4-phenylpiperidin-1-yl)piperidin-1-yl, 2-(4-piperidin-1-ylmethylphenyl)piperidin-1-yl, or 5-oxa-2-aza-bicyclo[2.2.1]heptan-2-yl (iii) when R is hydrogen, R¹ is 2,3-dichlorophenyl, then R² and R³ together with the nitrogen atom to which they are attached do not form 4-benzylpiperazin-1-yl, 2-phenylpyrrolidin-1-yl, 2-(4-piperidin-1-ylmethylphenyl)piperidin-1-yl, 2-(3-piperidin-1-ylmethylphenyl)piperidin-1-yl, 2-(4-piperidin-1-ylmethylphenyl)pyrrolidin-1-yl, 2-(3-piperidin-1-ylmethylphenyl)pyrrolidin-1-yl, or 2-phenylpiperidin-1-yl (iv) when R is hydrogen, R¹ is 4-methylphenyl, and R² is hydrogen then R³ is not 1-benzylpiperidin-4-yl, 2- 3-, and 4-methylcyclohexyl, 2- 3-, and 4-hydroxycyclohexyl, 2-benzyloxycyclohexyl, 2-ethyloxycarbonylcyclohexyl, tricyclo[3.3.1.1˜3,7]dec-1-yl, 3-hydroxytricyclo[3.3.1.1˜3,7]dec-1-yl, admant-1-yl, or 2- or 3- and (v) when R is hydrogen, R¹ is 2,5-dimethylphenyl, and R² is hydrogen then R³ is not 2-methylcyclohexyl.

In a third aspect, this invention is directed to a pharmaceutical composition comprising a compound of Formula (I) or a pharmaceutically acceptable salt thereof and a pharmaceutically acceptable excipient.

In a fourth aspect, this invention is directed to a method of treating a disease in a patient mediated the B1 receptor comprising administering to the patient a pharmaceutical composition comprising a therapeutically effective amount of a compound of Formula (I) or a pharmaceutically acceptable salt thereof and a pharmaceutically acceptable excipient. Specifically, the compounds of the present invention would be useful in the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

In a fifth aspect, this invention is directed to the use of one or more of the compounds of the present invention in the manufacture of a medicament for the treatment of a disorder mediated by B1 such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

### DETAILED DESCRIPTION OF THE INVENTION

### Definitions

Unless otherwise stated, the following terms used in the specification and claims are defined for the purposes of this Application and have the following meaning:

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means a —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or acyl and R′ is selected from hydrogen, alkyl, hydroxyalkyl, alkoxyalkyl, aryl, aralkyl, heteroaryl, heteroaralkyl, or haloalkyl e.g., aminomethyl, aminoethyl, 1,3-diaminopropyl, dimethylaminomethyl, diethylaminoethyl, acetylaminopropyl, and the like.

“Aminoalkoxy” means a —OR radical where R is aminoalkyl as defined above, e.g., 2-aminoethoxy, 2-dimethylaminopropoxy, and the like.

“Acyl” means a —C(O)R radical where R is alkyl, haloalkyl, aryl, aralkyl, heteroaryl, heteroaralkyl, heterocyclyl or heterocyclylalkyl as defined above, e.g., acetyl, trifluoroacetyl, benzoyl, and the like.

“Alkoxycarbonyl” means a —C(O)OR radical where R is alkyl as defined above, e.g., methoxycarbonyl, ethoxycarbonyl, and the like.

“Alkoxycarbonylalkyl” means a linear monovalent hydrocarbon radical of one to six carbon atoms or a branched monovalent hydrocarbon radical of three to six carbons substituted with at least one alkoxycarbonyl group, preferably one or two alkoxycarbonyl group, as defined above, e.g., 2-methoxycarbonylethyl, 1-, 2-, or 3-ethoxycarbonylpropyl, 2-ethoxycarbonylethyl, and the like.

“Aryl” means a monovalent monocyclic or bicyclic aromatic hydrocarbon radical of 6 to 12 ring atoms e.g., phenyl or naphthyl.

“Aralkyl” means -(alkylene)-R radical where R is aryl as defined above.

“Aryloxy” means a —OR radical where R is aryl as defined above, e.g., phenoxy, and the like.

“Aralkyloxy” means a —O-(alkylene)-R radical where R is aryl as defined above e.g., benzyloxy.

“Bridged cycloalkyl” refers to cycloalkyl rings as defined below of seven to ten rings atoms wherein two non-adjacent ring members are joined by an alkylene chain of one or two carbon atoms e.g.,

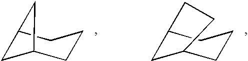

and the like.

“Bridged heterocyclyl” refers to heterocyclyl rings as defined below of seven to nine rings atoms wherein two non-adjacent ring members are joined by an alkylene chain of one or two carbon atoms e.g.,

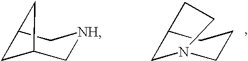

and the like.

Carboxyalkyl means a linear monovalent hydrocarbon radical of one to six carbon atoms or a branched monovalent hydrocarbon radical of three to six carbons substituted with at least one carboxy group, preferably one or two alkoxy groups, as defined above, e.g., 2-carboxyethyl, 1-, 2-, or 3-carboxypropyl, 2-carboxyethyl, and the like.

“Cycloalkyl” means a cyclic saturated or unsaturated monovalent hydrocarbon radical of three to ten carbon atoms and optionally fused to phenyl unless otherwise stated, e.g., cyclopropyl, cyclobutyl, cyclopentyl, or cyclohexyl.

“Cycloalkylalkyl” means -(alkylene)-R radical where R is cycloalkyl as defined above; e.g., cyclopropylmethyl, cyclobutylmethyl, cyclopentylethyl, or cyclohexylmethyl, and the like.

“Cyanoalkyl” means a linear monovalent hydrocarbon radical of one to six carbon atoms or a branched monovalent hydrocarbon radical of three to six carbons substituted with at least one cyano group, preferably one or two cyano group, as defined above, e.g., 2-cyanoethyl, 1-, 2-, or 3-cyanopropyl, and the like.

“Disubstituted amino” means —NRR′ where R and R′ is selected are independently selected from alkyl, acyl, aminoalkyl, hydroxyalkyl, alkoxyalkyl, aryl, aralkyl, heteroaryl, heteroaralkyl, or haloalkyl as defined herein. When R and R′ are alkyl, the group is also referred to herein as dialkylamino.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means a —OR radical where R is haloalkyl as defined above e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means a —OR radical where R is hydroxyalkyl as defined above.

“Heterocyclyl” means a saturated or unsaturated monovalent cyclic group of 3 to 10 ring atoms in which one or two ring atoms are heteroatom selected from —CO—, N, O, or S(O)ₙ, where n is an integer from 0 to 2, preferably N, O, or S(O)ₙ, the remaining ring atoms being C and wherein one or two ring carbon atoms can optionally be replaced by a —CO— group. More specifically the term heterocyclyl includes, but is not limited to, pyrrolidino, piperidino, morpholino, piperazino, tetrahydropyranyl, tetrahydroquinolinyl and thiomorpholino, and the like. When the heterocylyl ring is monocyclic it is also referred to herein as monocyclic heterocylyl ring.

“Heterocyclylalkyl” means a -(alkylene)-R radical where R is heterocycloalkyl ring as defined above e.g., piperazinylmethyl, morpholinylethyl, and the like.

“Heterocyclyloxy” means a —OR radical where R is heterocyclyl as defined above, e.g., piperazinyloxy, pyrrolidinyloxy, and the like.

“Heterocyclylalkyloxy” means a —O-(alkylene)-R radical where R is heterocyclyl as defined above e.g., piperidinylmethyloxy, piperazinylmethyloxy, and the like.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. More specifically the term heteroaryl includes, but is not limited to, pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, benzothiophenyl, benzthiazolyl, quinolinyl, isoquinolinyl, benzofuranyl, benzopyranyl, and thiazolyl, and the like.

“Heteroaralkyl” means a -(alkylene)-R radical where R is heteroaryl as defined above.

“Heteroaryloxy” means a —OR radical where R is heteroaryl as defined above, e.g., pyridinyloxy, furanyloxy, thienyloxy, and the like.

“Heteroaralkyloxy” means a —O-(alkylene)-R radical where R is heteroaralkyl as defined above e.g., pyridinmethyloxy, furanmethyloxy, and the like.

“Monosubstituted amino” means —NHR′ where R′ is selected from hydrogen, alkyl, acyl, hydroxyalkyl, aminoalkyl, alkoxyalkyl, aryl, aralkyl, heteroaryl, heteroaralkyl, or haloalkyl as defined herein. When R′ is alkyl, the group is also referred to herein as alkylamino.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolaamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Additionally, as used herein the terms alkyl includes all the possible isomeric forms of said alkyl group albeit only a few examples are set forth. Furthermore, when the cyclic groups such as aryl, heteroaryl, heterocycloalkyl are substituted, they include all the positional isomers albeit only a few examples are set forth. Furthermore, all polymorphic forms and hydrates of a compound of Formula (I) are within the scope of this invention.

“Optional” or “optionally” means that the subsequently described event or circumstance may but need not occur, and that the description includes instances where the event or circumstance occurs and instances in which it does not. For example, “heterocycloalkyl group optionally mono- or di-substituted with an alkyl group” means that the alkyl may but need not be present, and the description includes situations where the heterocycloalkyl group is mono- or disubstituted with an alkyl group and situations where the heterocycloalkyl group is not substituted with the alkyl group.

“Spiro heterocycloamino” means a saturated or unsaturated bicyclic group of 7 to 12 ring atoms where the rings are connected through just one atom and in which one, two, or three ring atoms are heteroatom selected from —O—, N, O, or S(O)ₙ, where n is an integer from 0 to 2, the remaining ring atoms being C provided that the bicyclic group contains at least one nitrogen ring atom.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

The expression “where the aryl, heteroaryl, or heterocyclyl ring in R⁴, R⁵, and R⁶ is optionally substituted with R^{d}, R^{e}, and R^{f} . . . ” and similar terms in the claims means that the all the rings mentioned, whether they are directly attached or are a part of another group e.g., aralkyl, heteroaralkyl, acyl, mono or disubstituted amino, etc., are optionally substituted. Additionally, the rings can be mono-, di-, or tri-substituted as indicated in the claims.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

Representative compounds of Formula (I) where R² is H and other groups are as listed in Table 1 below are:

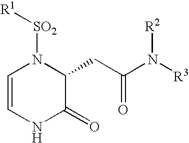

TABLE I

| Cpd # | R¹ | 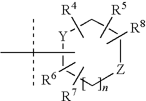 |
|---|---|---|
| 1 | 3,4-diClphenyl | 3-azabicyclo[3.2.2]non-3-yl |
| 2 | 4-methylphenyl | (1R,2R,4R)-1,7,7-trimethylbicyclo[2.2.1]hept-2- |
| | | yl and (1R,2S,4R)-1,7,7- |
| | | trimethylbicyclo[2.2.1]hept-2-yl |
| 3 | 4-methylphenyl | 1-(phenyl)-piperidin-4-yl |
| 4 | 4-methylphenyl | (1S,2S)-2-tert-butylcyclohexyl/(1R,2S)-2-tert- |
| | | butylcyclohexyl/(1S,2R)-2-tert-butylcyclohexyl/ |
| | | (1R,2R)-2-tert-butylcyclohexyl |
| 5 | 4-methylphenyl | 3-(R)-1-(benzyl)pyrrolidin-3-yl and 3-(S)-1- |
| | | (benzyl)pyrrolidin-3-yl |
| 6 | 4-methylphenyl | 3(R)-1-azabicyclo[2.2.2]oct-3-yl |
| 7 | 4-methylphenyl | 1-(pyrimidin-2-yl)piperidin-4-yl |
| 8 | 4-methylphenyl | 3-(S)-1-(tert-butyoxycarbonyl)piperidin-3-yl and |
| | | 3-(R)-1-(tert-butyoxycarbonyl)piperidin-3-yl |
| 9 | 4-methylphenyl | 1-(pyridin-4-yl)piperidin-4-yl |
| 10 | 4-methylphenyl | 3-(R)-1-(benzyl)piperidin-3-yl and 3-(S)-1- |
| | | (benzyl)piperidin-3-yl |
| 11 | 4-methylphenyl | 3-(R)-1-(2-propyl)piperidin-3-yl and 3-(S)-1-(2- |
| | | propyl)piperidin-3-yl |
| 12 | 4-methylphenyl | 3-(S)-1-(tert-butyoxycarbonyl)piperidin-4-yl |
| 13 | 4-methylphenyl | 1-(pyridin-4-ylmethyl)piperidin-4-yl |
| 14 | 4-methylphenyl | 3-(R)-1-(pyridin-4-ylmethyl)piperidin-3-yl and |
| | | 3-(S)-1-(pyridin-4-ylmethyl)piperidin-3-yl |
| 15 | 4-methylphenyl | 4-(R)-1-(tert-butyoxycarbonyl)-3,3-dimethyl- |
| | | piperidin-4-yl and 4-(S)-1-(tert-butyoxycarbonyl)- |
| | | 3,3-dimethyl-piperidin-4-yl |
| 16 | 4-methylphenyl | 1-methylpiperidin-4-yl |
| 17 | 4-methylphenyl | 4-(S)-3,3-dimethylpiperidin-4-yl and 4-(R)-3,3- |
| | | dimethylpiperidin-4-yl |
| 18 | 4-methylphenyl | 4-1-(acetyl)-3,3-dimethylpiperidin-4-yl |
| 19* | 4-methylphenyl | 4-(S)-1-(isobutyl)-3,3-dimethylpiperidin-4-yl |
| 20* | 4-methylphenyl | 4-(R)-1-(isobutyl)-3,3-dimethylpiperidin-4-yl |
| 21* | 4-methylphenyl | 4-(S)-1-benzyl-3,3-dimethylpiperidin-4-yl |
| 22* | 4-methylphenyl | 4-(R)-1-(benzyl)-3,3-dimethylpiperidin-4-yl |
| 23 | 4-methylphenyl | 1,3,3-trimethylpiperidin-4-yl |
| 24 | 4-methylphenyl | 4-(R)-1-(2-propyl)-3,3-dimethylpiperidin-4-yl and |
| | | 4-(S)-1-(2-propyl)-3,3-dimethylpiperidin-4-yl |
| 25 | 4-methylphenyl | 4-(R)-1-(pyridin-4-ylmethyl)-3,3- |
| | | dimethylpiperidin-4-yl and 4-(S)-1-(pyridin-4- |
| | | ylmethyl)-3,3-dimethylpiperidin-4-yl |
| 26* | 4-methylphenyl | 4-(S)-1-(pyridin-2-ylmethyl)-3,3- |
| | | dimethylpiperidin-4-yl |
| 27* | 4-methylphenyl | 4-(R)-1-(pyridin-2-ylmethyl)-3,3- |
| | | dimethylpiperidin-4-yl |
| 28 | 4-methylphenyl | 1-(benzoyl)piperidin-4-yl |
| 29 | 4-methylphenyl | 4-(R)-3,3-dimethyltetrahydropyran-4-yl and |
| | | 4-(S)-3,3-dimethyltetrahydropyran-4-yl |
| 30 | 4-methylphenyl | 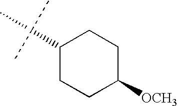 |
| 31 | 4-methylphenyl | 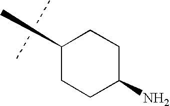 |
| 32 | 4-methylphenyl | 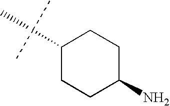 |
| 33 | 2,3- dichlorophenyl | 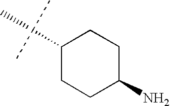 |
| 34 | 4-methylphenyl | (1R,3R,5S)-8-methyl-8-aza-bicyclo[3.2.1]octan- |
| | | 3-yl and (1S,3R,5S)-8-methyl-8-aza- |
| | | bicyclo[3.2.1]octan-3-yl |
| 35 | 4-methylphenyl | 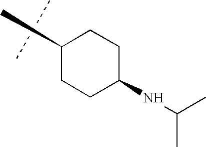 |
| 36 | 4-methylphenyl | 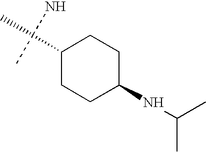 |
| 37 | 4-methylphenyl | 4-(R) and 4-(S)-2,2-dimethyl-4-oxocyclohexyl |
| 38 | 4-methylphenyl | 2,3-dimethylcyclohexyl |
| 39 | 4-methylphenyl | 4-methylpiperazin-1-yl |
| 40 | 4-methylphenyl | 2-cyclohexylcyclohexyl |
| 41 | 4-methylphenyl | 2,2-dimethyl-4-(3-fluoropiperidin-1-yl)cyclohexyl |
| 42 | 4-methylphenyl | 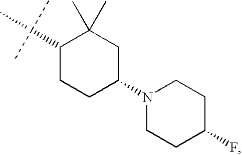 |
| | | 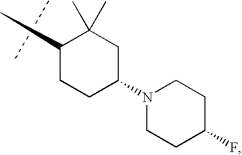 |
| | | 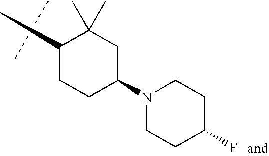 |
| | | 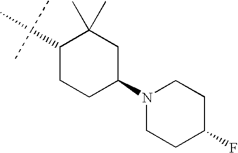 |
| 43 | 4-methylphenyl | 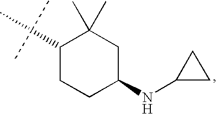 |
| | | 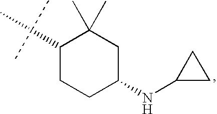 |
| | | 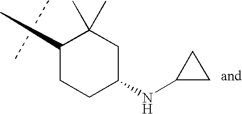 |
| | | 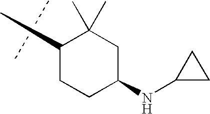 |
| 44 | 4-methylphenyl | 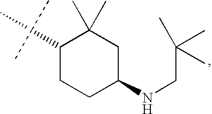 |
| | | 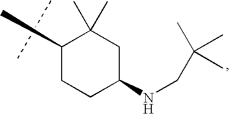 |
| | | 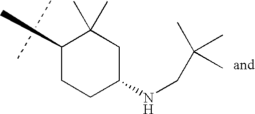 |
| | | 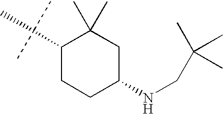 |
| 45 | 4-methylphenyl | 4(R)-3,3-dimethyl-1-cyclopropylpiperidin-4-yl |
| | | and 4(S)-3,3-dimethyl-1-cyclopropylpiperidin-4- |
| | | yl |
| 46 | 4-methylphenyl | 4(R)-3,3-dimethyl-1-(2,2,2- |
| | | trifluoroethyl)piperidin-4-yl and 4(S)-3,3- |
| | | dimethyl-1-(2,2,2-trifluoroethyl)-piperidin-4-yl |
| 47 | 2,3- | 4(R)-3,3-dimethylpiperidin-4-yl and 4(S)-3,3- |
| | dichlorophenyl | dimethylpiperidin-4-yl |
| 48 | 2,5-dimethyl-4- | (4S)-3,3-dimethylpiperidin-4-yl and (4R)-3,3- |
| | chlorophenyl | dimethylpiperidin-4-yl |
| 49 | 3,4- | 4(R)-3,3-dimethylpiperidin-4-yl and 4(S)-3,3- |
| | dichlorophenyl | dimethylpiperidin-4-yl |
| 50 | 4-methylphenyl | 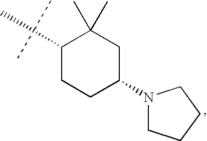 |
| | | 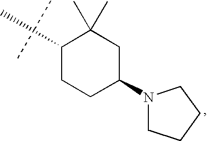 |
| | | 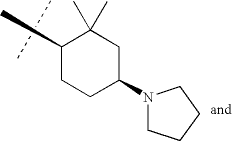 |
| | | 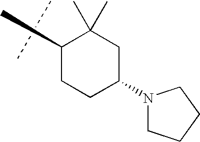 |
| 51 | 4-methylphenyl | 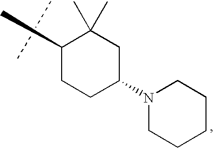 |
| | | 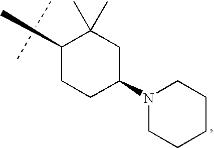 |
| | | 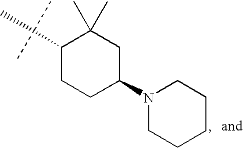 |
| | | 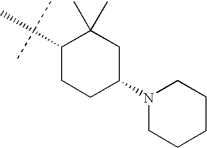 |
| 52 | 4-methylphenyl | 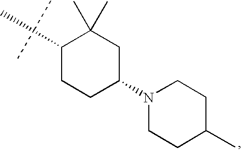 |
| | | 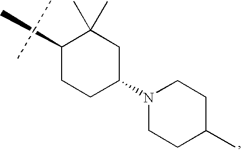 |
| | | 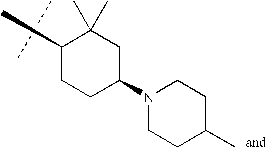 |
| | | 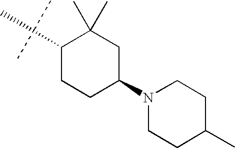 |
| 53 | 4-methylphenyl | 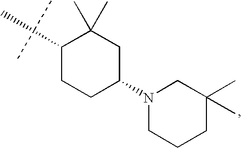 |
| | | 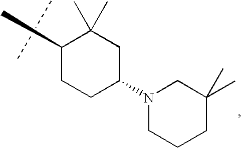 |
| | | 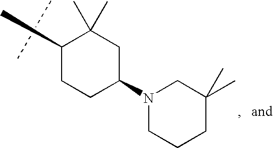 |
| | | 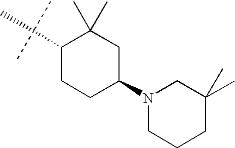 |
| 54 | 4-methylphenyl | 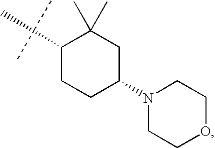 |
| | | 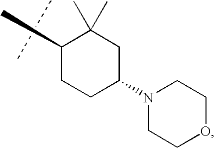 |
| | | 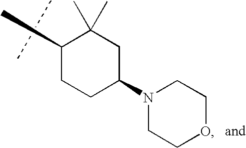 |
| | | 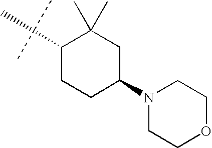 |
| 55 | 4-methylphenyl | 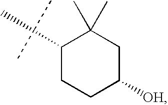 |
| | | 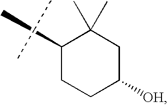 |
| | | 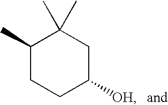 |
| | | 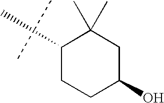 |
| 56* | 4-methylphenyl | 4-(S)-3,3-dimethylpiperidin-4-yl |
| 57* | 4-methylphenyl | 4-(R)-3,3-dimethylpiperidin-4-yl |
| 58 | 4-methylphenyl | 1-(2-phenethyl)-piperidin-4-yl |
| 59* | 4-methylphenyl | 4-(S)-3,3-dimethyltetrahydropyran-4-yl |
| 60* | 4-methylphenyl | 4-(R)-3,3-dimethyltetrahydropyran-4-yl |
| 61 | 4-methylphenyl | 3-(R)-piperidin-3-yl and 3-(S)-piperidin-3-yl |
| 62 | 4-methylphenyl | 1-(tert-butyoxycarbonyl)-4-methylpiperidin-4-yl |
| 63 | 4-methylphenyl | 4-methylpiperidin-4-yl |
| 64 | 4-methylphenyl | 3-(hydroxymethyl)bicyclo[2.2.1]heptan-2-yl |
| 65 | 4-methylphenyl | (1S,2S)-2-(2-hydroxypropan-2-yl)cyclohexyl/(1R, |
| | | 2S)-2-(2-hydroxypropan-2-yl)cyclohexyl/(1S,2R)- |
| | | 2-(2-hydroxypropan-2-yl)cyclohexyl/(1R,2R)-2- |
| | | tert-butylcyclohexyl |
| 66 | 4-methylphenyl | 1-tert-butoxycarbonylpiperidin-4-yl |
| 67 | 4-methylphenyl | 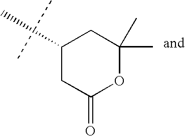 |
| | | 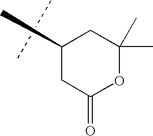 |
| 68 | 4-methylphenyl | 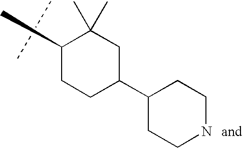 |
| | | 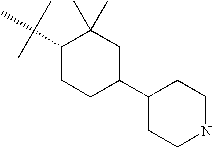 |
| 69 | 4-methylphenyl | (1S,2S)-2-hydroxymethylcyclohexyl and |
| | | (1R,2R)-2-hydroxymethylcyclohexyl |
| 70 | 4-methylphenyl | 3(R)-and 3(S)-1-tert-butoxycarbonylpiperazin-3-yl |
| 71 | 4-methylphenyl | tetrahydropyran-4-yl |
| 72 | 4-methylphenyl | 4(R)-3,3-dimethyl-1-(isobutyl)piperidin-4-yl |
| 73 | 4-chloro-2,5- | 4(R)-azepan-4-yl and 4(S)-azepan-yl |
| | dimethylphenyl | |
| 74 | 4-methylphenyl | 4(R)-azepan-4-yl and 4(S)-azepan-4-yl |
| 75 | 2,3-diClphenyl | 3(R)-pyrrolidin-3-yl |
| 76 | 4-methylphenyl | 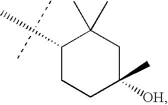 |
| | | 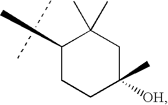 |
| | | 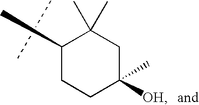 |
| | | 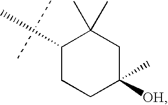 |
| 77 | 4-methylphenyl | (1,2,2,6,6-pentamethyl)piperidin-4-yl |
| 78 | 4-methylphenyl | 4(R)-1-methylazepan-4-yl and 4(S)-1- |
| | | methylazepan-4-yl |
| 79 | 4-methylphenyl | 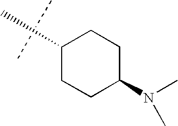 |
| 80 | 2,3-diClphenyl | 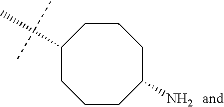 |
| | | 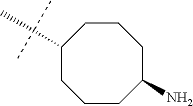 |
| 81* | 4-methylphenyl | 3,3-dimethyl-1-(pyridin-4-ylmethyl)-4(R)- |
| | | piperidin-4-yl |
| 82* | 4-methylphenyl | 3,3-dimethyl-1-(pyridin-4-ylmethyl)-4(S)- |
| | | piperidin-4-yl |
| 83* | 4-methylphenyl | 3,3-dimethyl-1-(pyridin-3-ylmethyl)-4(R)- |
| | | piperidin-4-yl |
| 84* | 4-methylphenyl | 3,3-dimethyl-1-(pyridin-3-ylmethyl)-4(S)- |
| | | piperidin-4-yl |
| 85 | 2,3-diClphenyl | 4-methylpiperazin-1-yl |
| 86 | 4-methylphenyl | 4,4-difluorocyclohex-1-yl |
| 87 | 2,5-dimethyl-4- | 4-methylpiperazin-1-yl |
| | chlorophenyl | |
| 88* | 4-methylphenyl | 4-(S)-3,3-dimethyl-1-tert- |
| | | butoxycarbonylpiperidin-4-yl |
| 89* | 4-methylphenyl | 4-(R)-3,3-dimethyl-1-tert- |
| | | butoxycarbonylpiperidin-4-yl |
| 90 | 4-methylphenyl | 1-cyclopropylpiperidin-4-yl |
| 91 | 4-methylphenyl | 4(R)-1-cyclopropylazepan-4-yl or 4(S)-1- |
| | | cyclopropylazepan-4-yl |
| 92 | 2,5-dimethyl-4- | 4-(R)-1-cyclopropylazepan-4-yl or 4-(S)-1- |
| | chlorophenyl | cyclopropyl-azepan-4-yl |
| 93* | 2,5-dimethyl-4- | 4-(S)-3,3 -dimethylpiperidin-4-yl |
| | chlorophenyl | |
| 94* | 4-methylphenyl | 4-(S)-1,3,3-trimethylpiperidin-4-yl |
| 95* | 4-methylphenyl | 4-(S)-1-ethyl-3,3-dimethylpiperidin-4-yl |
| 96* | 4-methylphenyl | 4-(S)-1-n-propyl-3,3-dimethylpiperidin-4-yl |
| 97* | 4-methylphenyl | 4-(S)-2,2-dimethylpropyl-3,3-dimethylpiperidin- |
| | | 4-yl |
| 98* | 4-methylphenyl | 4-(S)-1-cyclobutyl-3,3-dimethylpiperidin-4-yl |
| 99* | 4-methylphenyl | 4-(S)-1-cyclopentyl-3,3-dimethylpiperidin-4-yl |
| 100* | 2,3-dichloro- | 4-(S)-1-tert-butoxycarbonyl-3,3- |
| | phenyl | dimethylpiperidin-4-yl |
| 101* | 2,5-dimethyl-4- | 4-(S)-1,3,3-trimethylpiperidin-4-yl |
| | chlorophenyl | |
| 102* | 2,3-dichloro- | 4-(S)-3,3-dimethylpiperidin-4-yl |
| | phenyl | |
| 103* | 2,3-dichloro- | 4-(S)-1,3,3-trimethylpiperidin-4-yl |
| | phenyl | |
| 104* | 4-methylphenyl | 4-(S)-1-tert-butylcarbonyl-3,3-dimethylpiperidin- |
| | | 4-yl |
| 105 | 4-methylphenyl | 5-azocan-5-yl |
| 106* | 2,5-dimethyl-4- | 4-(S)-1-tert-butylcarbonyl-3,3-dimethylpiperidin- |
| | chlorophenyl | 4-yl |
| 107 | 4-methylphenyl | 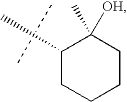 |
| | | 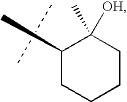 |
| | | 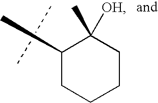 |
| | | 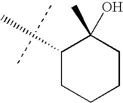 |
| 108 | 4-methylphenyl | 1-(pyridin-4-yl)piperidin-4-yl |
| 109 | 4-methylphenyl | 1-(pyridin-4-ylmethyl)piperidin-4-yl |
| 100 | 4-methylphenyl | piperidin-1-yl |
| 111 | 4-methylphenyl | 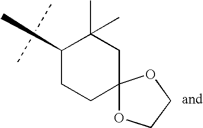 |
| | | 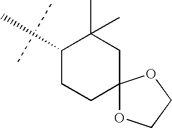 |
| 112* | 4-methylphenyl | 5(R)-6-oxopiperidin-2-yl |
| 113* | 4-methylphenyl | 5(S)-6-oxopiperidin-2-yl |
| 114 | 4-Cl-2,5- | 4-(R) and 4(S)-1-tert-butoxycarbonylazepan-4-yl |
| | diCH₃phenyl | |
| 115* | 4-methylphenyl | 4(S)-3,3-dimethyl-1,4′-bipiperidin-4-yl |
| 116* | 4-methylphenyl | 4(R)-3,3-dimethyl-1,4′-bipiperidin-4-yl |
| 117* | 2,3-dichloro- | 4(R)-1-cyclopropyl-3,3-dimethy1-4-piperidinyl |
| | phenyl | |
| 118* | 2,3-dichloro- | 4(S)-1-cyclopropyl-3,3-dimethyl-4-piperidinyl |
| | phenyl | |
| 119 | 4-methylphenyl | 4(S) and 4(R)-3,3-dimethyl-1-(2- |
| | | pyridinylmethyl)-4-piperidinyl |
| 120* | 4-methylphenyl | 4(R)-3,3-dimethyl-1-(2-(methyloxy)ethyl)-4- |
| | | piperidinyl |
| 121 | 4-methylphenyl | 4(5) and 4(R)-3,3-dimethy1-1-(cyclopropyl)-4- |
| | | piperidinyl n-oxide |

Note:
In Table 1, in compounds with * next to the compound number, the stereochemistry at the 4- position (chiral center) was arbitually assigned based on chiral HPLC separation of the diastereomers or stereospecific syntheses. The absolute stereochemistry was not confirmed unequivocally.

Representative compounds of Formula (I) where R¹, R² and R³ are as listed in Table II below are:

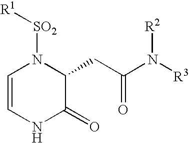

TABLE II

| Cpd. # | R¹ | R² | 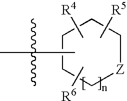 |
|---|---|---|---|
| 1 | 4-methylphenyl | methyl | 1-methylpiperidin-4-yl |
| 2 | 2,3- | methyl | 1-methylpiperidin-4-yl |
| | dichlorophenyl | | |
| 3 | 3,4- | methyl | 1-methylpiperidin-4-yl |
| | dichlorophenyl | | |
| 4 | 2,5-dimethyl-4- | methyl | 1-methylpiperidin-4-yl |
| | chlorophenyl | | |
| 5 | 4-methylphenyl | methyl | (1R,4R)-4-aminocyclohexyl and |
| | | | (1S,4R)-4-aminocyclohexyl and |
| 6 | 4-methylphenyl | methyl | 4(R)-3,3-dimethylpiperidin-4-yl |
| | | | and 4(S)-3,3-dimethylpiperidin-4- |
| | | | yl |
| 7 | 4-methylphenyl | methyl | 4(R)-3,3-dimethyl-1-cyclopropyl- |
| | | | piperidin-4-yl and 4(S)-3,3- |
| | | | dimethyl-1-cyclopropyl-piperidin- |
| | | | 4-yl |
| 8 | 4-methylphenyl | ethyl | 1-tert-butoxycarbonylpiperidin-4-yl |
| 9 | 4-methylphenyl | methyl | piperidin-4-yl |
| 10 | 4-methylphenyl | ethyl | piperidin-4-yl |
| 11 | 4-methylphenyl | n-propyl | piperidin-4-yl |
| 12 | 2,5-dimethyl-4- | methyl | 1-methylpiperidin-1-yl |
| | chlorophenyl | | |
| 13 | 4-methylphenyl | methyl | (1-tert-butoxycarbonyl)piperidin-4- |
| | | | yl |
| 14 | 4-methylphenyl | n-propyl | (1-tert-butoxycarbonyl)piperidin-4- |
| | | | yl |
| 15 | 4-methylphenyl | 2-meth- | (1-methyl)piperidin-4-yl |
| | | oxyethyl | |
| 16 | 4-methylphenyl | methyl | 4-(R)- and 4-(S)-1-tert- |
| | | | butoxycarbonylazepan-4-yl |
| 17 | 4-methylphenyl | ethyl | 4-(R)- and 4-(S)-1-tert- |
| | | | butoxycarbonylazepan-4-yl |
| 18 | 4-methylphenyl | methyl | 4-(R)- and 4-(S)-azepan-4-yl |
| 19 | 4-methylphenyl | ethyl | 4-(R)- and 4-(S)-azepan-4-yl |
| 20 | 4-methylphenyl | cyclo- | (1-tert-butoxycarbonyl)piperidin-1- |
| | | propyl | yl |
| 21 | 4-methylphenyl | cyclo- | 4-piperidinyl |
| | | propyl | |
| 22 | 4-methylphenyl | methyl | 1-methyl-4-piperidinyl |

Representative compounds of Formula (I) where R¹ is as listed in Table III and R² and R³ together with the nitrogen atom to which they are attached forms a group as listed in Table III below are:

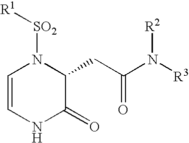

TABLE III

| Cpd.# | R¹ | —NR²R³ |
|---|---|---|
| 1 | 4-methylphenyl | 4-(4-trifluoromethylphenyl)piperidin-1-yl |
| 2 | 4-methylphenyl | 2-(4-methoxyphenyl)thiomorpholin-4-yl |
| 3 | 4-methylphenyl | 2-(4-chlorophenyl)pyrrolidin-1-yl |
| 4 | 2,3-diClphenyl | 3(S)-dimethylaminopyrrolidin-1-yl |
| 5 | 2,3-diClphenyl | 3(R)-dimethylaminopyrrolidin-1-yl |
| 6 | 4-methylphenyl | 3(S)-dimethylaminopyrrolidin-1-yl |
| 7 | 2,5-dimethyl-4- | 3(R)-dimethylaminopyrrolidin- l-yl |
| | chlorophenyl | |
| 8 | 2,5-dimethyl-4- | 4-(piperidin-1-yl)piperidin-1-yl |
| | chlorophenyl | |
| 9 | 2,5-dimethyl-4- | 4-dimethylaminopiperidin-1-yl |
| | chlorophenyl | |
| 10 | 4-methylphenyl | 3-(S)-amino-4(R)phenylpyrrolidin-1-yl |
| 11 | 2,5-dimethyl-4- | 3(S)-pyrrolidin-1-ylpiperdin-1-yl |
| | chlorophenyl | |
| 22 | 2,5-dimethyl-4- | 3(S)-pyrrolidin-1-ylpyrrolidin-1-yl |
| | chlorophenyl | |
| 13 | 2,5-dimethyl-4- | 3(S)-dimethylaminopiperidin-1-yl |
| | chlorophenyl | |
| 14 | 2,5-dimethyl-4- | 3(S)-dimethylaminoazepan-1-yl |
| | chlorophenyl | |
| 15 | 2,5-dimethyl-4- | 4-hydroxymethylpiperidin-1-yl |
| | chlorophenyl | |
| 16 | 2,5-dimethyl-4- | 3(S)-hydroxymethylpiperidin-1-yl and |
| | chlorophenyl | 3(R)-hydroxymethylpiperidin-1-yl |
| 17 | 2,5-dimethyl-4- | 2(S)-hydroxymethylpiperidin-1-yl and |
| | chlorophenyl | 2(R)-hydroxymethylpiperidin-1-yl |
| 18 | 2,5-dimethyl-4- | 4-(pyrrolidin-1-ylmethyl)piperidin-1-yl |
| | chlorophenyl | |
| 19 | 2,5-dimethyl-4- | 3(R)-(pyrrolidin-1-ylmethyl)piperidin-1-yl |
| | chlorophenyl | and 3(S)-(pyrrolidin-1-ylmethyl)piperidin-1-yl |
| 20 | 2,5-dimethyl-4- | 3(S)-aminopyrrolidin-1-yl |
| | chlorophenyl | |
| 21 | 2,5-dimethyl-4- | 4(R)-(dimethylamino)azepan-1-yl and |
| | chlorophenyl | 4(S)-(dimethylamino)azepan-1-yl |
| 22 | 2,5-dimethyl-4- | 3(R)-dimethylaminomethylpiperidin-1-yl |
| | chlorophenyl | and 3(S)-dimethylaminomethylpiperidin-1-yl |
| 23 | 2,5-dimethyl-4- | 3-(S)-(pyrro1idin-1-yl)azepan-1-yl |
| | chlorophenyl | |
| 24 | 2,5-dimethyl-4- | 3(S)-methyl-4(R)-pyrrolidin-1-yl]- |
| | chlorophenyl | pyrrolidin-1-yl; |
| | | 3(S)-methyl-4(S)-pyrrolidin-1-yl]- |
| | | pyrrolidin-1-yl; |
| | | 3(R)-methyl-4(R)-pyrrolidin-1-yl]- |
| | | pyrrolidin-1-yl; or |
| | | 3(R)-methyl-4(S)-pyrrolidin-1-yl]- |
| | | pyrrolidin-1-yl |
| 25 | 2,5-dimethyl-4- | 3-(S)-(pyrrolidin-1-ylmethyl)pyrrolidin-1-yl or |
| | chlorophenyl | 3(R)-(pyrrolidin-1-ylmethyl)pyrrolidin- 1-yl |
| 26 | 2,5-dimethyl-4- chlorophenyl | 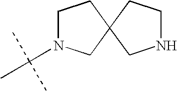 |
| 27 | 2,3-dichloro- | 4-(R)-dimethylaminoazepan-1-yl or 4-(S)- |
| | phenyl | dimethylaminoazepan-1-yl |
| 28 | 2,5-dimethyl-4- | 4′-(R)-N-cycloproyl-N-methylamin- |
| | chlorophenyl | oazepan-1-yl or 4-(S)-N-cyclopropyl-N- |
| | | methylaminoazepan-1-yl |
| 29 | 2,5-dimethyl-4- | 4-(S)-hydroxy-3,3-dimnethylpiperidin-1-yl or |
| | chlorophenyl | 4-(R)-hydroxy-3,3-dimethylpiperidin-1-yl |
| 30 | 2,5-dimethyl-4- | 4-(S)-pyrrolidin-1-yl-3,3-dimethyl- |
| | chlorophenyl | piperidin-1-yl or 4-(R)-pyrrolidin-1-yl-3,3- |
| | | dimethyl-piperidin-1-yl |
| 31 | 2,5-dimethyl-4- | 3-(R)-dimethylaminopiperidin-1-yl |
| | chlorophenyl | |
| 32 | 2,5-dimethyl-4- chlorophenyl | 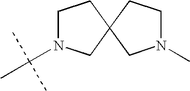 |
| 33 | 2,5-dimethyl-4- | 4-(R)-dimethylaminoazocan-1-yl or 4-(S)- |
| | chlorophenyl | dimethylaminoazocan-1-yl |
| 34 | 2,3-dichloro- | 4-(R)-dimethylaminoazocan-1-yl or 4-(S)- |
| | phenyl | dimethylaminoazocan-1-yl |
| 35 | 2,3-diClphenyl | 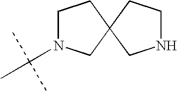 |
| 36 | 2,3-diClphenyl | 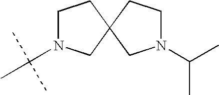 |
| 37 | 2,5-dimethyl-4- chlorophenyl | 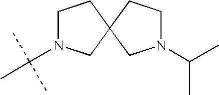 |
| 38 | 2,5-dimethyl-4- chlorophenyl | 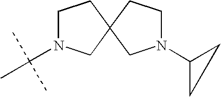 |
| 39 | 2,3-diClphenyl | 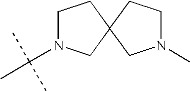 |
| 40 | 4-methylphenyl | 4-(benzoylamino)piperidin-1-yl |
| 41 | 4-methylphenyl | 4-isopropylaminopiperidin-1-yl |
| 42 | 4-methylphenyl | 2-benzylpyrrolidin-1-yl |
| 43 | 4-methylphenyl | 2-phenylpyrrolidin-1-yl |
| 44 | 4-methylphenyl | 4-phenoxypiperidin-1-yl |
| 45 | 4-methylphenyl | 3-oxopiperazin-1-yl |
| 46 | 4-methylphenyl | 2-methoxymethylpyrrolidin-1-yl |
| 47 | 4-methylphenyl | 2-(4-methoxybenzyl)pyrrolidin-1-yl |
| 48 | 4-methylphenyl | 4-(4-methylphenyl)piperidin-1-yl |
| 49 | 4-methylphenyl | 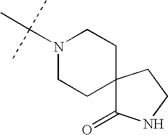 |
| 50 | 4-methylphenyl | 2-(indol-2-yl)pyrrolidin-1-yl |
| 51 | 4-methylphenyl | 4-(2-Cl-phenoxy)piperidin-1-yl |
| 52 | 4-methylphenyl | 2-(phenyl)thiomorpholin-4-yl |
| 53 | 4-methylphenyl | 2-(phenyl)morpholin-4-yl |
| 54 | 4-methylphenyl | 4-(indol-3-yl)piperidin-1-yl |
| 55 | 4-methylphenyl | 2-(2-methylphenyl)pyrrolidin-1-yl |
| 56 | 2,3-diClphenyl | 4-methylazepin-1-yl |
| 57 | 2,3-diClphenyl | 3-R-dimethylaminopyrrolidin-1-yl |

The compounds in Tables I, II, and III are named as follows but may not necessarily follow the compound numbering above:
(3R)-3-(2-(3-azabicyclo[3.2.2]non-3-yl)-2-oxoethyl)-4-((3,4-dichlorophenyl)-sulfonyl)-3,4-dihydro-2(1H)-pyrazinone;
2-((2R)-1-((4-methylphenyl)sulfonyl)-3-oxo-1,2,3,4-tetrahydro-2-pyrazinyl)-N-((1R,2R,4R)-1,7,7-trimethylbicyclo[2.2.1]hept-2-yl)acetamide; and 2-((2R)-1-((4-methylphenyl)sulfonyl)-3-oxo-1,2,3,4-tetrahydro-2-pyrazinyl)-N-((1R,2S,4R)-1,7,7-trimethylbicyclo[2.2.1]hept-2-yl)acetamide;
2-((2R)-1-((4-methylphenyl)sulfonyl)-3-oxo-1,2,3,4-tetrahydro-2-pyrazinyl)-N-(1-phenyl-4-piperidinyl)acetamide;
N-((1S,2S)-2-(1,1-dimethylethyl)cyclohexyl)-2-((2R)-1-((4-methylphenyl)sulfonyl)-3-oxo-1,2,3,4-tetrahydro-2-pyrazinyl)acetamide; N-((1R,2R)-2-(1,1-dimethylethyl)cyclohexyl)-2-((2R)-1-((4-methylphenyl)-sulfonyl)-3-oxo-1,2,3,4-tetrahydro-2-pyrazinyl)acetamide; N-((1S,2R)-2-(1,1-dimethylethyl)cyclohexyl)-2-((2R)-1-((4-methylphenyl)-sulfonyl)-3-oxo-1,2,3,4-tetrahydro-2-pyrazinyl)acetamide; and N-((1R,2S)-2-(1,1-dimethylethyl)cyclohexyl)-2-((2R)-1-((4-methylphenyl)-sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-((3S)-1-(phenylmethyl)-3-pyrrolidinyl)acetamide; and 2-((2R)-1-((4-methylphenyl)sulfonyl)-3-oxo-1,2,3,4-tetrahydro-2-pyrazinyl)-N-((3R)-1-(phenylmethyl)-3-pyrrolidinyl)acetamide;
N-((3R)-1-azabicyclo[2.2.2]oct-3-yl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(2-pyrimidinyl)-4-piperidinyl)acetamide;
1,1-dimethylethyl (3R)-3-((((2R)-1-((4-methylphenyl)sulfonyl)-3-oxo-1,2,3,4-tetrahydro-2-pyrazinyl)acetyl)amino)-1-piperidinecarboxylate; and 1,1-dimethylethyl (3S)-3-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(1-(4-pyridinyl)-4-piperidinyl)acetamide;
2-((2R)-1-((4-methylphenyl)sulfonyl)-3-oxo-1,2,3,4-tetrahydro-2-pyrazinyl)-N-((3R)-1-(phenylmethyl)-3-piperidinyl)acetamide; and 2-((2R)-1-((4-methylphenyl)sulfonyl)-3-oxo-1,2,3,4-tetrahydro-2-pyrazinyl)-N-((3S)-1-(phenylmethyl)-3-piperidinyl)acetamide;
N-((3R)-1-(1-methylethyl)-3-piperidinyl)-2-((2R)-1-((4-methylphenyl)sulfonyl)-3-oxo-1,2,3,4-tetrahydro-2-pyrazinyl)acetamide; and N-((3S)-1-(1-methylethyl)-3-piperidinyl)-2-((2R)-1-((4-methylphenyl)sulfonyl)-3-oxo-1,2,3,4-tetrahydro-2-pyrazinyl)acetamide;
1,1-dimethylethyl 4-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(1-(4-pyridinylmethyl)-4-piperidinyl)acetamide;
2-((2R)-1-((4-methylphenyl)sulfonyl)-3-oxo-1,2,3,4-tetrahydro-2-pyrazinyl)-N-((3S)-1-(4-pyridinylmethyl)-3-piperidinyl)acetamide; and 2-((2R)-1-((4-methylphenyl)sulfonyl)-3-oxo-1,2,3,4-tetrahydro-2-pyrazinyl)-N-((3R)-1-(4-pyridinylmethyl)-3-piperidinyl)acetamide;
1,1-dimethylethyl (4R)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate; and 1,1-dimethylethyl (4S)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(1-methyl-4-piperidinyl)acetamide;
N-((4S)-3,3-dimethyl-4-piperidinyl)-2-((2R)-1-((4-methylphenyl)sulfonyl)-3-oxo-1,2,3,4-tetrahydro-2-pyrazinyl)acetamide; and N-((4R)-3,3-dimethyl-4-piperidinyl)-2-((2R)-1-((4-methylphenyl)sulfonyl)-3-oxo-1,2,3,4-tetrahydro-2-pyrazinyl)acetamide;
N-(1-acetyl-3,3-dimethyl-4-piperidinyl)-2-((2R)-1-((4-methylphenyl)sulfonyl)-3-oxo-1,2,3,4-tetrahydro-2-pyrazinyl)acetamide;
N-((4S)-3,3-dimethyl-1-(2-methylpropyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-methylpropyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(phenylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(phenylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1,3,3-trimethyl-4-piperidinyl)acetamide;
N-((4R)-3,3-dimethyl-1-(1-methylethyl)-4-piperidinyl)-2-((2R)-1-((4-methyl-phenyl)sulfonyl)-3-oxo-1,2,3,4-tetrahydro-2-pyrazinyl)acetamide; and N-((4S)-3,3-dimethyl-1-(1-methylethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(4-pyridinylmethyl)-4-piperidinyl)-2-((2R)-1-((4-methylphenyl)sulfonyl)-3-oxo-1,2,3,4-tetrahydro-2-pyrazinyl)acetamide; and N-((4S)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(phenylcarbonyl)-4-piperidinyl)acetamide;
N-((4S)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide; and N-((4R)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-(trans-4-(methyloxy)cyclohexyl)-2-((2R)-1-((4-methylphenyl)sulfonyl)-3-oxo-1,2,3,4-tetrahydro-2-pyrazinyl)acetamide;
N-(cis-4-aminocyclohexyl)-2-((2R)-1-((4-methylphenyl)sulfonyl)-3-oxo-1,2,3,4-tetrahydro-2-pyrazinyl)acetamide;
N-(trans-4-aminocyclohexyl)-2-((2R)-1-((4-methylphenyl)sulfonyl)-3-oxo-1,2,3,4-tetrahydro-2-pyrazinyl)acetamide;
N-(trans-4-aminocyclohexyl)-2-((2R)-1-((2,3-dichlorophenyl)sulfonyl)-3-oxo-1,2,3,4-tetrahydro-2-pyrazinyl)acetamide;
N-((3-endo)-8-methyl-8-azabicyclo[3.2.1]oct-3-yl)-2-((2R)-1-((4-methylphenyl)-sulfonyl)-3-oxo-1,2,3,4-tetrahydro-2-pyrazinyl)acetamide; and N-((3-exo)-8-methyl-8-azabicyclo[3.2.1]oct-3-yl)-2-((2R)-1-((4-methylphenyl)-sulfonyl)-3-oxo-1,2,3,4-tetrahydro-2-pyrazinyl)acetamide;
N-(cis-4-((1-methylethyl)amino)cyclohexyl)-2-((2R)-1-((4-methylphenyl)sulfonyl)-3-oxo-1,2,3,4-tetrahydro-2-pyrazinyl)acetamide;
N-(trans-4-((1-methylethyl)amino)cyclohexyl)-2-((2R)-1-((4-methylphenyl)-sulfonyl)-3-oxo-1,2,3,4-tetrahydro-2-pyrazinyl)acetamide;
N-((1R)-2,2-dimethyl-4-oxocyclohexyl)-2-((2R)-1-((4-methylphenyl)sulfonyl)-3-oxo-1,2,3,4-tetrahydro-2-pyrazinyl)acetamide; and N-((1S)-2,2-dimethyl-4-oxocyclohexyl)-2-((2R)-1-((4-methylphenyl)sulfonyl)-3-oxo-1,2,3,4-tetrahydro-2-pyrazinyl)acetamide;
N-(2,3-dimethylcyclohexyl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(4-methyl-1-piperazinyl)acetamide;
N-1,1′-bi(cyclohexyl)-2-yl-2-((2R)-1-((4-methylphenyl)sulfonyl)-3-oxo-1,2,3,4-tetrahydro-2-pyrazinyl)acetamide;
N-(4-(3-fluoro-1-piperidinyl)-2,2-dimethylcyclohexyl)-2-((2R)-1-((4-methyl-phenyl)sulfonyl)-3-oxo-1,2,3,4-tetrahydro-2-pyrazinyl)acetamide;
N-((1R,4S)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1R,4R)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1S,4S)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide;
N-((1S,4R)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide; N-((1S,4S)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide; N-((1R,4R)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide; and N-((1R,4S)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide;
N-((1R,4S)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; N-((1R,4R)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; N-((1S,4S)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-((2,2-dimethylpropyl)amino)-2,2-dimethylcyclohexyl)-2′-((2R)-1-((4-methylphenyl)sulfonyl)-3-oxo-1,2,3,4-tetrahydro-2-pyrazinyl)acetamide;
N-((4S)-1-cyclopropyl-3,3-dimethyl-4-piperidinyl)-2-((2R)-1-((4-methylphenyl)-sulfonyl)-3-oxo-1,2,3,4-tetrahydro-2-pyrazinyl)acetamide; and N-((4R)-1-cyclopropyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4R)-3,3-dimethyl-1-(2,2,2-trifluoroethyl)-4-piperidinyl)-2-((2R)-1-((4-methyl-phenyl)sulfonyl)-3-oxo-1,2,3,4-tetrahydro-2-pyrazinyl)acetamide; and N-((4S)-3,3-dimethyl-1-(2,2,2-trifluoroethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-((4S)-3,3-dimethyl-4-piperidinyl)acetamide; and 2-((2R)-1-((2,3-dichlorophenyl)sulfonyl)-3-oxo-1,2,3,4-tetrahydro-2-pyrazinyl)-N-((4R)-3,3-dimethyl-4-piperidinyl)acetamide;
2-((2R)-1-((4-chloro-2,5-dimethylphenyl)sulfonyl)-3-oxo-1,2,3,4-tetrahydro-2-pyrazinyl)-N-((4S)-3,3-dimethyl-4-piperidinyl)acetamide; and 2-((2R)-1-((4-chloro-2,5-dimethylphenyl)sulfonyl)-3-oxo-1,2,3,4-tetrahydro-2-pyrazinyl)-N-((4R)-3,3-dimethyl-4-piperidinyl)acetamide;
2-((2R)-1-((3,4-dichlorophenyl)sulfonyl)-3-oxo-1,2,3,4-tetrahydro-2-pyrazinyl)-N-((4R)-3,3-dimethyl-4-piperidinyl)acetamide; and 2-((2R)-1-((3,4-dichlorophenyl)sulfonyl)-3-oxo-1,2,3,4-tetrahydro-2-pyrazinyl)-N-((4S)-3,3-dimethyl-4-piperidinyl)acetamide;
N-((1R,4S)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; and N-((1S,4R)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide;
N-((1R,4S)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide; and N-((1S,4R)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide;
N-((1S,4R)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; N-((1S,4S)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; N-((1R,4R)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; and N-((1R,4S)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide;
N-((1R,4S)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1R,4R)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1S,4S)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide;
N-((1S,4R)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; and N-((1R,4S)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide;
N-((1S,4R)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; N-((1S,4S)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; N-((1R,4R)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; and N-((1R,4S)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide;
N-((4S)-3,3-dimethyl-4-piperidinyl)-2-((2R)-1-((4-methylphenyl)sulfonyl)-3-oxo-1,2,3,4-tetrahydro-2-pyrazinyl)acetamide; 123
N-((4R)-3,3-dimethyl-4-piperidinyl)-2-((2R)-1-((4-methylphenyl)sulfonyl)-3-oxo-1,2,3,4-tetrahydro-2-pyrazinyl)acetamide;
N-(1-(2-phenethyl)-4-piperidinyl)-2-((2R)-1-((4-methylphenyl)sulfonyl)-3-oxo-1,2,3,4-tetrahydro-2-pyrazinyl)acetamide;
N-((4S)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-((4R)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-((3R)-3-piperidinyl)-2-((2R)-1-((4-methylphenyl)sulfonyl)-3-oxo-1,2,3,4-tetrahydro-2-pyrazinyl)acetamide; and N-((3S)-3-piperidinyl)-2-((2R)-1-((4-methylphenyl)sulfonyl)-3-oxo-1,2,3,4-tetrahydro-2-pyrazinyl)acetamide;
1,1-dimethylethyl 4-methyl-4-((((2R)-1-((4-methylphenyl)sulfonyl)-3-oxo-1,2,3,4-tetrahydro-2-pyrazinyl)acetyl)amino)-1-piperidinecarboxylate;
N-(4-methyl-4-piperidinyl)-2-((2R)-1-((4-methylphenyl)sulfonyl)-3-oxo-1,2,3,4-tetrahydro-2-pyrazinyl)acetamide;
N-(3-(hydroxymethyl)bicyclo[2.2.1]hept-2-yl)-2-((2R)-1-((4-methylphenyl)sulfonyl)-3-oxo-1,2,3,4-tetrahydro-2-pyrazinyl)acetamide;
N-((1RS,2RS)-2-(1-hydroxy-1-methylethyl)cyclohexyl)-2-((2R)-1-((4-methylphenyl)sulfonyl)-3-oxo-1,2,3,4-tetrahydro-2-pyrazinyl)acetamide;
1,1-dimethylethyl 4-((((2R)-1-((4-methylphenyl)sulfonyl)-3-oxo-1,2,3,4-tetrahydro-2-pyrazinyl)acetyl)amino)-1-piperidinecarboxylate;
N-((4RS)-2,2-dimethyl-6-oxotetrahydro-2H-pyran-4-yl)-2-((2R)-1-((4-methylphenyl)sulfonyl)-3-oxo-1,2,3,4-tetrahydro-2-pyrazinyl)acetamide;
N-((4RS)-3,3-dimethyl-1,4′-bipiperidin-4-yl)-2-((2R)-1-((4-methylphenyl)sulfonyl)-3-oxo-1,2,3,4-tetrahydro-2-pyrazinyl)acetamide;
N-((1RS,2RS)-2-(hydroxymethyl)cyclohexyl)-2-((2R)-1-((4-methylphenyl)sulfonyl)-3-oxo-1,2,3,4-tetrahydro-2-pyrazinyl)acetamide;
1,1-dimethylethyl (3RS)-3-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(tetrahydro-2H-pyran-4-yl)acetamide;
N-((4R)-3,3-dimethyl-1-(2-methylpropyl)-4-piperidinyl)-2-((2R)-1-((4-methylphenyl)sulfonyl)-3-oxo-1,2,3,4-tetrahydro-2-pyrazinyl)acetamide;
N-((4R/S)-4-azepanyl)-2-((2R)-1-((4-chloro-2,5-dimethylphenyl)sulfonyl)-3-oxo-1,2,3,4-tetrahydro-2-pyrazinyl)acetamide trifluoroacetic acid salt;
N-((4R)-4-azepanyl)-2-((2R)-1-((4-methylphenyl)sulfonyl)-3-oxo-1,2,3,4-tetrahydro-2-pyrazinyl)acetamide and N-((4S)-4-azepanyl)-2-((2R)-1-((4-methylphenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-((3R)-3-pyrrolidinyl)acetamide;
N-((1R/S,4R/S)-4-hydroxy-2,2,4-trimethylcyclohexyl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1,2,2,6,6-pentamethyl-4-piperidinyl)acetamide;
N-((4R/S)-1-methyl-4-azepanyl)-2-((2R)-1-((4-methylphenyl)sulfonyl)-3-oxo-1,2,3,4-tetrahydro-2-pyrazinyl)acetamide;
N-(trans-4-(dimethylamino)cyclohexyl)-2-((2R)-1-((4-methylphenyl)sulfonyl)-3-oxo-1,2,3,4-tetrahydro-2-pyrazinyl)acetamide;
N-(cis/trans-5-aminocyclooctyl)-2-((2R)-1-((2,3-dichlorophenyl)sulfonyl)-3-oxo-1,2,3,4-tetrahydro-2-pyrazinyl)acetamide;
N-((4R)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(3-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(3-pyridinylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-(4-methyl-1-piperazinyl)acetamide 3,3,3-trifluoropropanoic acid salt;
N-(4,4-difluorocyclohexyl)-2-((2R)-1-((4-methylphenyl)sulfonyl)-3-oxo-1,2,3,4-tetrahydro-2-pyrazinyl)acetamide;
2-((2R)-1-((4-chloro-2,5-dimethylphenyl)sulfonyl)-3-oxo-1,2,3,4-tetrahydro-2-pyrazinyl)-N-(4-methyl-1-piperazinyl)acetamide;
1,1-dimethylethyl (4S)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
1,1-dimethylethyl (4R)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
N-(1-cyclopropyl-4-piperidinyl)-2-((2R)-1-((4-methylphenyl)sulfonyl)-3-oxo-1,2,3,4-tetrahydro-2-pyrazinyl)acetamide;
N-((4R/S)-1-cyclopropyl-4-azepanyl)-2-((2R)-1-((4-methylphenyl)sulfonyl)-3-oxo-1,2,3,4-tetrahydro-2-pyrazinyl)acetamide;
2-((2R)-1-((4-chloro-2,5-dimethylphenyl)sulfonyl)-3-oxo-1,2,3,4-tetrahydro-2-pyrazinyl)-N-((4RS)-1-cyclopropyl-4-azepanyl)acetamide trifluoroacetic acid salt;
2-((2R)-1-((4-chloro-2,5-dimethylphenyl)sulfonyl)-3-oxo-1,2,3,4-tetrahydro-2-pyrazinyl)-N-((4S)-3,3-dimethyl-4-piperidinyl)acetamide;
2-((2R)-1-((4-methylphenyl)sulfonyl)-3-oxo-1,2,3,4-tetrahydro-2-pyrazinyl)-N-((4S)-1,3,3-trimethyl-4-piperidinyl)acetamide;
N-((4S)-1-ethyl-3,3-dimethyl-4-piperidinyl)-2-((2R)-1-((4-methylphenyl)sulfonyl)-3-oxo-1,2,3,4-tetrahydro-2-pyrazinyl)acetamide;
N-((4S)-3,3-dimethyl-1-propyl-4-piperidinyl)-2-((2R)-1-((4-methylphenyl)sulfonyl)-3-oxo-1,2,3,4-tetrahydro-2-pyrazinyl)acetamide;
N-((4S)-1-(2,2-dimethylpropyl)-3,3-dimethyl-4-piperidinyl)-2-((2R)-1-((4-methylphenyl)sulfonyl)-3-oxo-1,2,3,4-tetrahydro-2-pyrazinyl)acetamide;
N-((4S)-1-cyclobutyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4S)-1-cyclopentyl-3,3-dimethyl-4-piperidinyl)-2-((2R)-1-((4-methylphenyl)-sulfonyl)-3-oxo-1,2,3,4-tetrahydro-2-pyrazinyl)acetamide;
1,1-dimethylethyl (4S)-4-((((2R)-1-((2,3-dichlorophenyl)sulfonyl)-3-oxo-1,2,3,4-tetrahydro-2-pyrazinyl)acetyl)amino)-3,3-dimethyl-1-piperidinecarboxylate;
2-((2R)-1-((4-chloro-2,5-dimethylphenyl)sulfonyl)-3-oxo-1,2,3,4-tetrahydro-2-pyrazinyl)-N-((4S)-1,3,3-trimethyl-4-piperidinyl)acetamide;
2-((2R)-1-((2,3-dichlorophenyl)sulfonyl)-3-oxo-1,2,3,4-tetrahydro-2-pyrazinyl)-N-((4S)-3,3-dimethyl-4-piperidinyl)acetamide;
2-((2R)-1-((2,3-dichlorophenyl)sulfonyl)-3-oxo-1,2,3,4-tetrahydro-2-pyrazinyl)-N-((4S)-1,3,3-trimethyl-4-piperidinyl)acetamide;
N-((4S)-1-(2,2-dimethylpropanoyl)-3,3-dimethyl-4-piperidinyl)-2-((2R)-1-((4-methylphenyl)sulfonyl)-3-oxo-1,2,3,4-tetrahydro-2-pyrazinyl)acetamide;
N-5-azocanyl-2-((2R)-1-((4-methylphenyl)sulfonyl)-3-oxo-1,2,3,4-tetrahydro-2-pyrazinyl)acetamide;
1,1-dimethylethyl (4S)-4-((((2R)-1-((4-chloro-2,5-dimethylphenyl)sulfonyl)-3-oxo-1,2,3,4-tetrahydro-2-pyrazinyl)acetyl)amino)-3,3-dimethyl-1-piperidinecarboxylate;
N-((1RS,2RS)-2-hydroxy-2-methylcyclohexyl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(4-pyridinyl)-4-piperidinyl)acetamide;
2-((2R)-1-((4-methylphenyl)sulfonyl)-3-oxo-1,2,3,4-tetrahydro-2-pyrazinyl)-N-(1-(4-pyridinylmethyl)-4-piperidinyl)acetamide;
2-((2R)-1-((4-methylphenyl)sulfonyl)-3-oxo-1,2,3,4-tetrahydro-2-pyrazinyl)-N-1-piperidinylacetamide;
N-((8RS)-7,7-dimethyl-1,4-dioxaspiro[4.5]dec-8-yl)-2-((2R)-1-((4-methylphenyl)-sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-((3S)-6-oxo-3-piperidinyl)acetamide;
2-((2R)-1-((4-methylphenyl)sulfonyl)-3-oxo-1,2,3,4-tetrahydro-2-pyrazinyl)-N-((3R)-6-oxo-3-piperidinyl)acetamide;
1,1-dimethylethyl (4RS)-4-((((2R)-1-((4-chloro-2,5-dimethylphenyl)sulfonyl)-3-oxo-1,2,3,4-tetrahydro-2-pyrazinyl)acetyl)amino)-1-azepanecarboxylate;
N-((4S)-3,3-dimethyl-1,4′-bipiperidin-4-yl)-2-((2R)-1-((4-methylphenyl)sulfonyl)-3-oxo-1,2,3,4-tetrahydro-2-pyrazinyl)acetamide;
N-((4R)-3,3-dimethyl-1,4′-bipiperidin-4-yl)-2-((2R)-1-((4-methylphenyl)sulfonyl)-3-oxo-1,2,3,4-tetrahydro-2-pyrazinyl)acetamide;
N-((4R)-1-cyclopropyl-3,3-dimethyl-4-piperidinyl)-2-((2R)-1-((2,3-dichloro-phenyl)sulfonyl)-3-oxo-1,2,3,4-tetrahydro-2-pyrazinyl)acetamide;
N-((4S)-1-cyclopropyl-3,3-dimethyl-4-piperidinyl)-2-((2R)-1-((2,3-dichloro-phenyl)sulfonyl)-3-oxo-1,2,3,4-tetrahydro-2-pyrazinyl)acetamide;
N-((4S)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-(methyloxy)ethyl)-4-piperidinyl)-2-((2R)-1-((4-methylphenyl)sulfonyl)-3-oxo-1,2,3,4-tetrahydro-2-pyrazinyl)acetamide;
N-((4S) and 4(R)-1-cyclopropyl-3,3-dimethyl-4-piperidinyl)-2-((2R)-1-((4-methylphenyl)sulfonyl)-3-oxo-1,2,3,4-tetrahydro-2-pyrazinyl)acetamide n-oxide;
2-((2R)-1-((4-methylphenyl)sulfonyl)-3-oxo-1,2,3,4-tetrahydro-2-pyrazinyl)-N-methyl-N-(1-methyl-4-piperidinyl)acetamide;
2-((2R)-1-((2,3-dichlorophenyl)sulfonyl)-3-oxo-1,2,3,4-tetrahydro-2-pyrazinyl)-N-methyl-N-(1-methyl-4-piperidinyl)acetamide;
2-((2R)-1-((3,4-dichlorophenyl)sulfonyl)-3-oxo-1,2,3,4-tetrahydro-2-pyrazinyl)-N-methyl-N-(1-methyl-4-piperidinyl)acetamide;
2-((2R)-1-((4-chloro-2,5-dimethylphenyl)sulfonyl)-3-oxo-1,2,3,4-tetrahydro-2-pyrazinyl)-N-methyl-N-(1-methyl-4-piperidinyl)acetamide;
N-(cis-4-aminocyclohexyl)-N-methyl-2-((2R)-1-((4-methylphenyl)sulfonyl)-3-oxo-1,2,3,4-tetrahydro-2-pyrazinyl)acetamide; and N-(trans-4-aminocyclohexyl)-N-methyl-2-((2R)-1-((4-methylphenyl)sulfonyl)-3-oxo-1,2,3,4-tetrahydro-2-pyrazinyl)acetamide;
N-((4R)-3,3-dimethyl-4-piperidinyl)-N-methyl-2-((2R)-1-((4-methylphenyl)-sulfonyl)-3-oxo-1,2,3,4-tetrahydro-2-pyrazinyl)acetamide; and N-((4S)-3,3-dimethyl-4-piperidinyl)-N-methyl-2-((2R)-1-((4-methylphenyl)-sulfonyl)-3-oxo-1,2,3,4-tetrahydro-2-pyrazinyl)acetamide;
N-((4RS)-1-cyclopropyl-3,3-dimethyl-4-piperidinyl)-N-methyl-2-((2R)-1-((4-methylphenyl)sulfonyl)-3-oxo-1,2,3,4-tetrahydro-2-pyrazinyl)acetamide;
1,1-dimethylethyl 4-(ethyl(((2R)-1-((4-methylphenyl)sulfonyl)-3-oxo-1,2,3,4-tetrahydro-2-pyrazinyl)acetyl)amino)-1-piperidinecarboxylate;
N-methyl-2-((2R)-1-((4-methylphenyl)sulfonyl)-3-oxo-1,2,3,4-tetrahydro-2-pyrazinyl)-N-4-piperidinylacetamide;
N-ethyl-2-((2R)-1-((4-methylphenyl)sulfonyl)-3-oxo-1,2,3,4-tetrahydro-2-pyrazinyl)-N-4-piperidinylacetamide;
N-propyl-2-((2R)-1-((4-methylphenyl)sulfonyl)-3-oxo-1,2,3,4-tetrahydro-2-pyrazinyl)-N-4-piperidinylacetamide;
2-((2R)-1-((4-chloro-2,5-dimethylphenyl)sulfonyl)-3-oxo-1,2,3,4-tetrahydro-2-pyrazinyl)-N-methyl-N-(1-methyl-4-piperidinyl)acetamide;
1,1-dimethylethyl 4-(methyl(((2R)-1-((4-methylphenyl)sulfonyl)-3-oxo-1,2,3,4-tetrahydro-2-pyrazinyl)acetyl)amino)-1-piperidinecarboxylate;
1,1-dimethylethyl 4-((((2R)-1-((4-methylphenyl)sulfonyl)-3-oxo-1,2,3,4-tetrahydro-2-pyrazinyl)acetyl)(propyl)amino)-1-piperidinecarboxylate;
N-(2-(methyloxy)ethyl)-2-((2R)-1-((4-methylphenyl)sulfonyl)-3-oxo-1,2,3,4-tetrahydro-2-pyrazinyl)-N-(1-methyl-4-piperidinyl)acetamide;
1,1-dimethylethyl (4RS)-4-(methyl(((2R)-1-((4-methylphenyl)sulfonyl)-3-oxo-1,2,3,4-tetrahydro-2-pyrazinyl)acetyl)amino)-1-azepanecarboxylate;
1,1-dimethylethyl (4R/S)-4-(ethyl(((2R)-1-((4-methylphenyl)sulfonyl)-3-oxo-1,2,3,4-tetrahydro-2-pyrazinyl)acetyl)amino)-1-azepanecarboxylate;
N-((4R/S)-4-azepanyl)-N-methyl-2-((2R)-1-((4-methylphenyl)sulfonyl)-3-oxo-1,2,3,4-tetrahydro-2-pyrazinyl)acetamide;
N-((4R/S)-4-azepanyl)-N-ethyl-2-((2R)-1-((4-methylphenyl)sulfonyl)-3-oxo-1,2,3,4-tetrahydro-2-pyrazinyl)acetamide;
1,1-dimethylethyl 4-(cyclopropyl(((2R)-1-((4-methylphenyl)sulfonyl)-3-oxo-1,2,3,4-tetrahydro-2-pyrazinyl)acetyl)amino)-1-piperidinecarboxylate;
N-cyclopropyl-2-((2R)-1-((4-methylphenyl)sulfonyl)-3-oxo-1,2,3,4-tetrahydro-2-pyrazinyl)-N-4-piperidinylacetamide;
N-methyl-2-((2R)-1-((4-methylphenyl)sulfonyl)-3-oxo-1,2,3,4-tetrahydro-2-pyrazinyl)-N-(1-methyl-4-piperidinyl)acetamide;
(3R)-3-(2-(2-(4-(methyloxy)phenyl)-4-thiomorpholinyl)-2-oxoethyl)-4-((4-methyl-phenyl)sulfonyl)-3,4-dihydro-2(1H)-pyrazinone;
(3R)-3-(2-(2-(4-chlorophenyl)-1-pyrrolidinyl)-2-oxoethyl)-4-((4-methylphenyl)-sulfonyl)-3,4-dihydro-2(1H)-pyrazinone;
(3R)-4-((2,3-dichlorophenyl)sulfonyl)-3-(2-((3S)-3-(dimethylamino)-1-pyrrolidinyl)-2-oxoethyl)-3,4-dihydro-2(1H)-pyrazinone;
(3R)-4-((2,3-dichlorophenyl)sulfonyl)-3-(2-((3R)-3-(dimethylamino)-1-pyrrolidinyl)-2-oxoethyl)-3,4-dihydro-2(1H)-pyrazinone;
(3R)-3-(2-((3S)-3-(dimethylamino)-1-pyrrolidinyl)-2-oxoethyl)-4-((4-methylphenyl)sulfonyl)-3,4-dihydro-2(1H)-pyrazinone;
(3R)-4-((4-chloro-2,5-dimethylphenyl)sulfonyl)-3-(2-((3R)-3-(dimethylamino)-1-pyrrolidinyl)-2-oxoethyl)-3,4-dihydro-2(1H)-pyrazinone;
(3R)-3-(2-(1,4′-bipiperidin-1′-yl)-2-oxoethyl)-4-((4-chloro-2,5-dimethylphenyl)-sulfonyl)-3,4-dihydro-2(1H)-pyrazinone;
(3R)-4-((4-chloro-2,5-dimethylphenyl)sulfonyl)-3-(2-(4-(dimethylamino)-1-piperidinyl)-2-oxoethyl)-3,4-dihydro-2(1H)-pyrazinone;
(3R)-3-(2-((3S)-3-(amino)-4(R)-4-phenyl-1-pyrrolidinyl)-2-oxoethyl)-4-((4-methylphenyl)sulfonyl)-3,4-dihydro-2(1H)-pyrazinone;
(3R)-4-((4-chloro-2,5-dimethylphenyl)sulfonyl)-3-(2-oxo-2-((3S)-3-(1-pyrrolidinyl)-1-piperidinyl)ethyl)-3,4-dihydro-2(1H)-pyrazinone;
(3R)-3-(2-((3′S)-1,3′-bipyrrolidin-1′-yl)-2-oxoethyl)-4-((4-chloro-2,5-dimethylphenyl)sulfonyl)-3,4-dihydro-2(1H)-pyrazinone;
(3R)-4-((4-chloro-2,5-dimethylphenyl)sulfonyl)-3-(2-((3S)-3-(dimethylamino)-1-piperidinyl)-2-oxoethyl)-3,4-dihydro-2(1H)-pyrazinone;
(3R)-4-((4-chloro-2,5-dimethylphenyl)sulfonyl)-3-(2-((3S)-3-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-(4-(hydroxymethyl)-1-piperidinyl)-2-oxoethyl)-3,4-dihydro-2(1H)-pyrazinone;
(3R/S)-4-((4-chloro-2,5-dimethylphenyl)sulfonyl)-3-(2-((3R)-3-(hydroxymethyl)-1-piperidinyl)-2-oxoethyl)-3,4-dihydro-2(1H)-pyrazinone;
(3R)-4-((4-chloro-2,5-dimethylphenyl)sulfonyl)-3-(2-((2R/S)-2-(hydroxymethyl)-1-piperidinyl)-2-oxoethyl)-3,4-dihydro-2(1H)-pyrazinone;
(3R)-4-((4-chloro-2,5-dimethylphenyl)sulfonyl)-3-(2-oxo-2-(4-(1-pyrrolidinylmethyl)-1-piperidinyl)ethyl)-3,4-dihydro-2(1H)-pyrazinone;
(3R)-4-((4-chloro-2,5-dimethylphenyl)sulfonyl)-3-(2-oxo-2-((3R/S)-3-(1-pyrrolidinylmethyl)-1-piperidinyl)ethyl)-3,4-dihydro-2(1H)-pyrazinone;
(3R)-3-(2-((3S)-3-amino-1-pyrrolidinyl)-2-oxoethyl)-4-((4-chloro-2,5-dimethyl-phenyl)sulfonyl)-3,4-dihydro-2(1H)-pyrazinone;
(3R)-4-((4-chloro-2,5-dimethylphenyl)sulfonyl)-3-(2-((4R/S)-4-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-((3R/S)-3-((dimethylamino)-methyl)-1-piperidinyl)-2-oxoethyl)-3,4-dihydro-2(1H)-pyrazinone;
(3R)-4-((4-chloro-2,5-dimethylphenyl)sulfonyl)-3-(2-oxo-2-((3S)-3-(1-pyrrolidinyl)-1-azepanyl)ethyl)-3,4-dihydro-2(1H)-pyrazinone;
(3R)-4-((4-chloro-2,5-dimethylphenyl)sulfonyl)-3-(2-((3′R/S,4′R/S)-4′-methyl-1,3′-bipyrrolidin-1′-yl)-2-oxoethyl)-3,4-dihydro-2(1H)-pyrazinone;
(3R)-4-((4-chloro-2,5-dimethylphenyl)sulfonyl)-3-(2-oxo-2-((3R/S)-3-(1-pyrrolidinyl-methyl)-1-pyrrolidinyl)ethyl)-3,4-dihydro-2(1H)-pyrazinone;
(3R)-4-((4-chloro-2,5-dimethylphenyl)sulfonyl)-3-(2-(2,7-diazaspiro[4.4]non-2-yl)-2-oxoethyl)-3,4-dihydro-2(1H)-pyrazinone;
(3R)-4-((2,3-dichlorophenyl)sulfonyl)-3-(2-((4R/S)-4-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-((4R/S)-4-(cyclopropyl(methyl)amino)-1-azepanyl)-2-oxoethyl)-3,4-dihydro-2(1H)-pyrazinone trifluoroacetic acid salt;
(3R)-4-((4-chloro-2,5-dimethylphenyl)sulfonyl)-3-(2-((4R/S)-4-hydroxy-3,3-dimethyl-1-piperidinyl)-2-oxoethyl)-3,4-dihydro-2(1H)-pyrazinone;
(3R)-4-((4-chloro-2,5-dimethylphenyl)sulfonyl)-3-(2-((4R/S)-3,3-dimethyl-4-(1-pyrrolidinyl)-1-piperidinyl)-2-oxoethyl)-3,4-dihydro-2(1H)-pyrazinone;
(3R)-4-((4-chloro-2,5-dimethylphenyl)sulfonyl)-3-(2-((3R)-3-(dimethylamino)-1-piperidinyl)-2-oxoethyl)-3,4-dihydro-2(1H)-pyrazinone trifluoroacetic acid salt;
(3R)-4-((4-chloro-2,5-dimethylphenyl)sulfonyl)-3-(2-(7-methyl-2,7-diazaspiro-[4.4]non-2-yl)-2-oxoethyl)-3,4-dihydro-2(1H)-pyrazinone;
(3R)-4-((4-chloro-2,5-dimethylphenyl)sulfonyl)-3-(2-((4R)-4-(dimethylamino)-1-azocanyl)-2-oxoethyl)-3,4-dihydro-2(1H)-pyrazinone;
(3R)-4-((2,3-dichlorophenyl)sulfonyl)-3-(2-((4R/S)-4-(dimethylamino)-1-azocanyl)-2-oxoethyl)-3,4-dihydro-2(1H)-pyrazinone;
(3R)-3-(2-(2,7-diazaspiro[4.4]non-2-yl)-2-oxoethyl)-4-((2,3-dichlorophenyl)sulfonyl)-3,4-dihydro-2(1H)-pyrazinone;
(3R)-4-((2,3-dichlorophenyl)sulfonyl)-3-(2-(7-(1-methylethyl)-2,7-diazaspiro[4.4]non-2-yl)-2-oxoethyl)-3,4-dihydro-2(1H)-pyrazinone;
(3R)-4-((4-chloro-2,5-dimethylphenyl)sulfonyl)-3-(2-(7-(1-methylethyl)-2,7-diazaspiro[4.4]non-2-yl)-2-oxoethyl)-3,4-dihydro-2(1H)-pyrazinone;
(3R)-4-((4-chloro-2,5-dimethylphenyl)sulfonyl)-3-(2-(7-cyclopropyl-2,7-diazaspiro[4.4]non-2-yl)-2-oxoethyl)-3,4-dihydro-2(1H)-pyrazinone;
(3R)-4-((2,3-dichlorophenyl)sulfonyl)-3-(2-(7-methyl-2,7-diazaspiro[4.4]non-2-yl)-2-oxoethyl)-3,4-dihydro-2(1H)-pyrazinone;
N-(1-(2-((2R)-1-((4-methylphenyl)sulfonyl)-3-oxo-1,2,3,4-tetrahydro-2-pyrazinyl)acetyl)-4-piperidinyl)benzamide;
(3R)-3-(2-(4-((1-methylethyl)amino)-1-piperidinyl)-2-oxoethyl)-4-((4-methylphenyl)sulfonyl)-3,4-dihydro-2(1H)-pyrazinone;
(3R)-4-((4-methylphenyl)sulfonyl)-3-(2-oxo-2-(2RS-(phenylmethyl)-1-pyrrolidinyl)ethyl)-3,4-dihydro-2(1H)-pyrazinone;
(3R)-4-((4-methylphenyl)sulfonyl)-3-(2-oxo-2-(2RS-phenyl-1-pyrrolidinyl)ethyl)-3,4-dihydro-2(1H)-pyrazinone;
(3R)-4-((4-methylphenyl)sulfonyl)-3-(2-oxo-2-(4-(phenyloxy)-1-piperidinyl)ethyl)-3,4-dihydro-2(1H)-pyrazinone;
(3R)-4-((4-methylphenyl)sulfonyl)-3-(2-oxo-2-(3-oxo-1-piperazinyl)ethyl)-3,4-dihydro-2(1H)-pyrazinone;
(3R)-3-(2-(2-RS-((methyloxy)methyl)-1-pyrrolidinyl)-2-oxoethyl)-4-((4-methyl-phenyl)sulfonyl)-3,4-dihydro-2(1H)-pyrazinone;
(3R)-3-(2-(2-RS-((4-(methyloxy)phenyl)methyl)-1-pyrrolidinyl)-2-oxoethyl)-4-((4-methylphenyl)sulfonyl)-3,4-dihydro-2(1H)-pyrazinone;
(3R)-3-(2-(4-(4-methylphenyl)-1-piperidinyl)-2-oxoethyl)-4-((4-methylphenyl)-sulfonyl)-3,4-dihydro-2(1H)-pyrazinone;
8-(((2R)-1-((4-methylphenyl)sulfonyl)-3-oxo-1,2,3,4-tetrahydro-2-pyrazinyl)acetyl)-2,8-diazaspiro[4.5]decan-1-one;
(3R)-3-(2-(2-RS-(1H-indol-2-yl)-1-pyrrolidinyl)-2-oxoethyl)-4-((4-methylphenyl)sulfonyl)-3,4-dihydro-2(1H)-pyrazinone;
(3R)-3-(2-(4-((2-chlorophenyl)oxy)-1-piperidinyl)-2-oxoethyl)-4-((4-methylphenyl)sulfonyl)-3,4-dihydro-2(1H)-pyrazinone;
(3R)-4-((4-methylphenyl)sulfonyl)-3-(2-oxo-2-(2-RS-phenyl-4-thiomorpholinyl)ethyl)-3,4-dihydro-2(1H)-pyrazinone;
(3R)-4-((4-methylphenyl)sulfonyl)-3-(2-oxo-2-(2-RS-phenyl-4-morpholinyl)ethyl)-3,4-dihydro-2(1H)-pyrazinone;
(3R)-3-(2-(4-(1H-indol-3-yl)-1-piperidinyl)-2-oxoethyl)-4-((4-methylphenyl)sulfonyl)-3,4-dihydro-2(1H)-pyrazinone;
(3R)-3-(2-(2-(2-RS-methylphenyl)-1-pyrrolidinyl)-2-oxoethyl)-4-((4-methylphenyl)sulfonyl)-3,4-dihydro-2(1H)-pyrazinone;
(3R)-4-((2,3-dichlorophenyl)sulfonyl)-3-(2-(4-methyl-1,4-diazepan-1-yl)-2-oxoethyl)-3,4-dihydro-2(1H)-pyrazinone; and
(3R)-4-((2,3-dichlorophenyl)sulfonyl)-3-(2-((3R)-3-(dimethylamino)-1-pyrrolidinyl)-2-oxoethyl)-3,4-dihydro-2(1H)-pyrazinone.

### EMBODIMENTS

While the broadest definition of this invention is set forth in the Summary of the Invention, certain compounds of Formula (I) and (aa) are preferred. For example,

A. One preferred group of compounds is that wherein R² is hydrogen.
B. Another preferred group of compounds is that wherein R² is alkyl, alkoxyalkyl, or cycloalkyl, preferably alkyl. More preferably, R² is 2-methoxyethyl, cyclopropyl, methyl, ethyl, propyl, or butyl, preferably methyl, ethyl, propyl, or butyl.
(i) Within the above groups A and B, and groups contained therein, one preferred group of compounds is that wherein R³ is bridged heterocyclyl optionally substituted with one, two or three substitutents independently selected from alkyl, alkoxy, hydroxyl, hydroxyalkyl, or alkoxyalkyl.
(ii) Within the above groups A and B, and groups contained therein, another preferred group of compounds is that wherein R³ is bridged cycloalkyl optionally substituted with one, two or three substitutents independently selected from alkyl, alkoxy, hydroxyl, hydroxyalkyl, or alkoxyalkyl.
(iii) Within the above groups A and B, and groups contained therein, yet another preferred group of compounds is that wherein R³ is cyclopentyl or cyclohexyl substituted with R⁴, R⁵, and R⁶ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano; and R⁷ and R⁸ are hydrogen. Preferably, R³ is a group of formula:

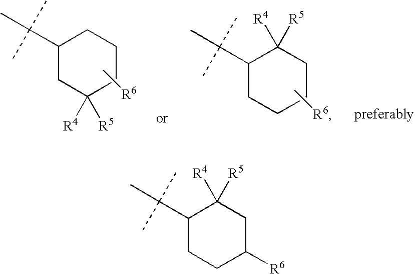

wherein R⁴ and R⁵ are independently hydrogen or alkyl, preferably hydrogen or methyl, even more preferably methyl and R⁶ is as defined immediately above. Preferably R⁶ is alkyl, halo, hydroxyl, alkoxy, hydroxyalkyl, aminoalkyl, monosubstituted or disubstituted amino, heteroaryl, heteroaralkyl, heterocyclyl or heterocyclylalkyl, optionally substituted with R^{d}, R^{e}, and R^{f} as defined in the Summary of the Invention. Preferably, R³ is 2-(2-hydroxymethyl)cyclohexyl, 2,2-dimethyl-4-(hydroxy)-4-methylcyclohexyl, 4-dimethylaminocyclohexyl, 4,4-difluorocyclohexyl, 2-hydroxy-2-methylcyclohexyl, 2-methylcyclohexyl, 4-hydroxycyclohexyl, benzyloxycyclohexyl, 2-(ethoxycarbonyl)-cyclohexyl, 2-(hydroxymethyl)cyclohexyl, 2-tert-butylcyclohexyl, 2-(2-hydroxypropan-2-yl)cyclohexyl, 4-methoxycyclohexyl, 4-aminocyclohexyl, 4-(isopropylamino)cyclohexyl, 2,3-dimethylcyclohexyl, 2,2-dimethyl-4-(3-fluoropiperidin-1-yl)cyclohexyl, 2,2-dimethyl-4-(4-fluoropiperidin-1-yl)cyclohexyl, 2,2-dimethyl-4-cyclopropylaminocyclohexyl, 2,2-dimethyl-4-(2,2-dimethylpropylamino)cyclohexyl, 2,2-dimethyl-4-pyrrolidin-1-ylcyclohexyl, 2,2-dimethyl-4-piperidin-1-ylcyclohexyl, 2,2-dimethyl-4-(4-methylpiperidin-1-yl)cyclohexyl, 2,2-dimethyl-4-(3,3-dimethylpiperidin-1-yl)cyclohexyl, 2,2-dimethyl-4-(morpholin-4-yl)cyclohexyl, 2,2-dimethyl-4-(hydroxy)cyclohexyl, or 2,2-dimethyl-4-piperidin-4-ylcyclohexyl. Preferably, R³ is 2-methylcyclohexyl, 4-hydroxycyclohexyl, benzyloxycyclohexyl, 2-(ethoxycarbonyl)-cyclohexyl, 2-(hydroxymethyl)cyclohexyl, 2-tert-butylcyclohexyl, 2-(2-hydroxypropan-2-yl)cyclohexyl, 4-methoxycyclohexyl, 4-aminocyclohexyl, 4-(isopropylamino)cyclohexyl, 2,3-dimethylcyclohexyl, 2,2-dimethyl-4-(3-fluoropiperidin-1-yl)cyclohexyl, 2,2-dimethyl-4-(4-fluoropiperidin-1-yl)cyclohexyl, 2,2-dimethyl-4-cyclopropylaminocyclohexyl, 2,2-dimethyl-4-2,2-dimethylpropylaminocyclohexyl, 2,2-dimethyl-4-pyrrolidin-1-ylcyclohexyl, 2,2-dimethyl-4-piperidin-1-ylcyclohexyl, 2,2-dimethyl-4-(4-methylpiperidin-1-yl)cyclohexyl, 2,2-dimethyl-4-(3,3-dimethylpiperidin-1-yl)cyclohexyl, 2,2-dimethyl-4-(morpholin-4-yl)cyclohexyl, 2,2-dimethyl-4-(hydroxy)cyclohexyl, or 2,2-dimethyl-4-piperidin-4-ylcyclohexyl.
(iv) Within the above groups A and B, and groups contained therein, yet another preferred group of compounds is that wherein R³ is:

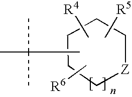

where n is 0, 1, 2 or 3 and Z is —NH— and R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano; and R⁷ and R⁸ are hydrogen. Preferably, R³ is piperidinyl, pyrrolidinyl, azocanyl, or azepanyl attached to the amido nitrogen via a carbon ring atom and substituted with R⁴ and R⁵ which are independently hydrogen or alkyl, and R⁶ are as defined immediately above. Preferably R⁶ is hydrogen, alkyl, haloalkyl, alkoxycarbonyl, cycloalkyl, cycloalkylalkyl, aralkyl, heteroaralkyl, heteroaryl, aryl,

acyl, aminoalkyl, or alkoxyalkyl. Preferably, R³ is

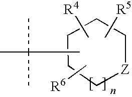

wherein R⁴ and R⁵ are independently hydrogen or alkyl, preferably hydrogen or methyl and R⁶ are as defined immediately above. Preferably R⁶ is hydrogen, alkyl, haloalkyl, alkoxycarbonyl, cycloalkyl, cycloalkylalkyl, aralkyl, heteroaralkyl, heteroaryl, aryl, acyl, aminoalkyl, or alkoxyalkyl optionally substituted with R^{d}, R^{e}, and R^{f} as defined in the Summary of the Invention. Preferably, R³ is 3,3-dimethylpiperidin-4-yl, 1-benzyl-3,3-dimethylpiperidin-4-yl, 1-methyl-3,3-dimethylpiperidin-4-yl, 1-(isobutyl)-3,3-dimethylpiperidin-4-yl, 1-(pyridin-2-ylmethyl)-3,3-dimethylpiperidin-4-yl, 1-(2-propyl)-3,3-dimethylpiperidin-4-yl, 1-(pyridin-4-ylmethyl)-3,3-dimethylpiperidin-4-yl, 1-(benzyl)-3,3-dimethylpiperidin-4-yl, 1-(acetyl)-3,3-dimethylpiperidin-4-yl, 1-(2-phenethyl)-piperidin-4-yl, 1-(phenyl)-piperidin-4-yl, 1-(pyridin-4-ylmethyl)piperidin-4-yl, 1-(pyridin-4-yl)piperidin-4-yl, 1-(benzyl)piperidin-3-yl, 1-(benzyl)pyrrolidin-3-yl, 1-(tert-butyoxy-carbonyl)piperidin-4-yl, 1-(pyridin-4-ylmethyl)piperidin-3-yl, 1-(methyl)piperidin-4-yl, piperidin-3-yl, 1-(tert-butyoxycarbonyl)-3,3-dimethyl-piperidin-4-yl, 1-(2-propyl)piperidin-3-yl, 1-(pyrimidin-2-yl)piperidin-4-yl, 1-(benzoyl)piperidin-4-yl, 3,4-dimethylpiperidin-4-yl, 1-cyclopropyl-3,3-dimethylpiperidin-4-yl, 1-(pyridin-3-ylmethyl)-3,3-dimethylpiperidin-4-yl, 1-(2-methoxyethyl)-3,3-dimethylpiperidin-4-yl, 3,3-dimethyl-1-(2,2,2-trifluoroethyl)piperidin-4-yl, 1-(tert-butyoxy-carbonyl)piperidin-3-yl, piperidin-3-yl, pyrrolidin-3-yl, 1-methylazepan-4-yl, 1-cyclopropylpiperidin-4-yl, 1-cyclopropylazepan-4-yl, 1-ethyl-3,3-dimethylpiperidin-4-yl, 1-n-propyl-3,3-dimethylpiperidin-4-yl, 1-2,2-dimethylpropyl-3,3-dimethylpiperidin-4-yl, 1-cyclobutyl-3,3-dimethylpiperidin-4-yl, 1-cyclopentyl-3,3-dimethylpiperidin-4-yl, azocan-5-yl, or 1-(tert-butyoxy-carbonyl)azepan-4-yl. Preferably, R³ is 3,3-dimethylpiperidin-4-yl, 1-benzyl-3,3-dimethylpiperidin-4-yl, 1-methyl-3,3-dimethylpiperidin-4-yl, 1-(isobutyl)-3,3-dimethylpiperidin-4-yl, 1-(pyridin-2-ylmethyl)-3,3-dimethylpiperidin-4-yl, 1-(2-propyl)-3,3-dimethylpiperidin-4-yl, 1-(pyridin-4-ylmethyl)-3,3-dimethylpiperidin-4-yl, 1-(benzyl)-3,3-dimethylpiperidin-4-yl, 1-(acetyl)-3,3-dimethylpiperidin-4-yl, 1-(2-phenethyl)-piperidin-4-yl, 1-(phenyl)-piperidin-4-yl, 1-(pyridin-4-ylmethyl)piperidin-4-yl, 1-(pyridin-4-yl)piperidin-4-yl, 1-(benzyl)piperidin-3-yl, 1-(benzyl)pyrrolidin-3-yl, 1-(tert-butyoxy-carbonyl)piperidin-4-yl, 1-(pyridin-4-ylmethyl)piperidin-3-yl, 1-(methyl)piperidin-4-yl, piperidin-3-yl, 1-(tert-butyoxycarbonyl)-3,3-dimethyl-piperidin-4-yl, 1-(2-propyl)piperidin-3-yl, 1-(pyrimidin-2-yl)piperidin-4-yl, 1-(benzoyl)piperidin-4-yl, 3,4-dimethylpiperidin-4-yl, 1-cyclopropyl-3,3-dimethylpiperidin-4-yl, 1-(pyridin-3-ylmethyl)-3,3-dimethylpiperidin-4-yl, or 1-(2-methoxyethyl)-3,3-dimethylpiperidin-4-yl. Preferably, R³ is 3,3-dimethylpiperidin-4-yl, 1-benzyl-3,3-dimethylpiperidin-4-yl, 1-methyl-3,3-dimethylpiperidin-4-yl, 1-(isobutyl)-3,3-dimethylpiperidin-4-yl, 1-(pyridin-2-ylmethyl)-3,3-dimethylpiperidin-4-yl, 1-(2-propyl)-3,3-dimethylpiperidin-4-yl, 1-(pyridin-4-ylmethyl)-3,3-dimethylpiperidin-4-yl, 1-(benzyl)-3,3-dimethylpiperidin-4-yl, 1-(acetyl)-3,3-dimethylpiperidin-4-yl, 1-(tert-butyoxycarbonyl)-3,3-dimethyl-piperidin-4-yl, 1-cyclopropyl-3,3-dimethylpiperidin-4-yl, 1-(pyridin-3-ylmethyl)-3,3-dimethylpiperidin-4-yl, or 1-(2-methoxyethyl)-3,3-dimethylpiperidin-4-yl.
(v) Within the above preferred groups A and B, and more preferred groups contained therein, yet another preferred group of compounds is that wherein R³ is a group of formula (a) where n is 1, Y is —CH₂—, and Z is O and R⁴, R⁵, and R⁶ are as defined in the Summary of the Invention and R⁷ and R⁸ are hydrogen. Preferably, R³ is:

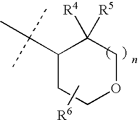

wherein n is 0, 1, or 2, preferably 0 or 1, R⁴ and R⁵ are independently hydrogen or alkyl, preferably hydrogen or methyl and R⁶ are as defined in the Summary of the Invention.
(vi) Within the above preferred groups A and B, and another more preferred groups contained therein, yet another preferred group of compounds is that wherein R³ is a group of formula (a) where n is 0, 1, or 2, Y is CH₂—, and Z is S or SO₂ and R⁴, R⁵, and R⁶ are as defined in the Summary of the Invention; and R⁷ and R⁸ are hydrogen. Preferably, R³ is

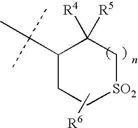

wherein R⁴ and R⁵ are independently hydrogen or alkyl, preferably hydrogen or methyl and R⁶ are as defined immediately above.
(vii) Within the above preferred groups A and B, and more preferred groups contained therein, yet another preferred group of compounds is that wherein R³ is a group of formula (a) where n is 1, Y is —CH₂—, and Z is —CONH— and R⁴, R⁵, and R⁶ are as defined in the Summary of the Invention; and R⁷ and R⁸ are hydrogen. Preferably, R³ is

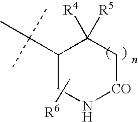

wherein R⁴ and R⁵ are independently hydrogen or alkyl, preferably hydrogen or methyl and R⁶ are as defined immediately above.
(viii) Within the above groups A and B, and groups contained therein, yet another preferred group of compounds is that wherein R³ is a group of formula (a) where R⁴ and R⁷ combine together with the carbon atom to which they are attached to form a saturated or unsaturated monocyclic (C₃-C₈)cycloalkyl optionally substituted with R^{d}, R^{e}, and R^{f} as defined in the Summary of the Invention, preferably R⁴ and R⁷ combine together with the carbon atom to which they are attached to form a saturated monocyclic C₃-C₈ cycloalkyl optionally substituted with R^{d}, R^{e}, and R^{f}. Preferably, R³ is

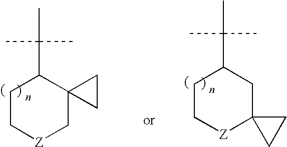

where n and Z are as defined in the Summary of the Invention, preferably n is 0, 1, or 2 and Z is —NR⁶— where R⁶ is as defined in the Summary of the Invention.
(ix) Within the above groups A and B, and groups contained therein, yet another preferred group of compounds is that wherein R³ is a group of formula (a) where R⁴ and R⁷ can combine together with the carbon atom to which they are attached to form a a saturated or unsaturated monocyclic heterocyclyl ring containing three to six ring atom wherein one or two ring atoms are selected from —C(O)—, —O—, —S—, —SO—, —SO₂—, or —NH— and wherein the heterocyclic ring is substituted with R^{m}, Rⁿ and R^{o} as defined in the Summary of the Invention. Preferably, R³ is:

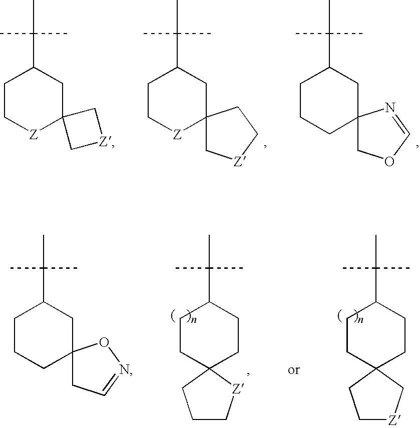

where Z is —CH₂—, —CO—, or —NH—, n is 0, 1, or 2, preferably 0 or 1, and Z′ is —NH—, —O—, or —SO₂—, and the rings are substituted with R⁶, R⁸, R^{m} and R^{o} as defined in the Summary of the Invention with R⁵ and Rⁿ being hydrogen.
C. Yet another preferred group of compounds is that wherein R² and R³ together with the nitrogen atom to which they are attached form monocyclic heterocyclyl substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R^{g}, R^{h}, and Rⁱ is optionally substituted with R^{j}, R^{k}, or R^{l} independently selected from alkyl, halo, alkoxy, haloalkyl, hydroxyl, carboxy, alkoxycarbonyl, haloalkoxy, or cyano provided that at least one of R^{g}, R^{h}, and Rⁱ is not hydrogen; preferably, R² and R³ together with the nitrogen atom to which they are attached form morpholin-4-yl, thiomorpholin-4-yl, pyrrolidin-1-yl, piperidin-1-yl, piperazin-1-yl, azepan-1-yl or azepan-1-yl, more preferably piperidin-1-yl, piperazin-1-yl, azepan-1-yl or azepan-1-yl, optionally substituted with the groups listed above, preferably R^{g}, R^{h}, and Rⁱ independently selected from hydrogen, alkyl, phenyl (optionally substituted with alkyl, haloalkyl, alkoxy, or halo), hydroxyl, phenoxy (optionally substituted with alkyl, haloalkyl, alkoxy, or halo), amino, monosubstituted amino, or disubstituted amino, heterocyclyl, hydroxyalkyl, alkoxyalkyl, aminoalkyl, or heterocyclylalkyl, more preferably amino, monosubstituted amino, or disubstituted amino; and R⁷ and R⁸ are hydrogen.
D. Yet another preferred group of compounds is that wherein R² and R³ together with the nitrogen atom to which they are attached form spiro heterocycloamino each of which is substituted with R^{g}, R^{h}, and Rⁱ as defined in the Summary of the Invention. Preferably, R² and R³ together form:

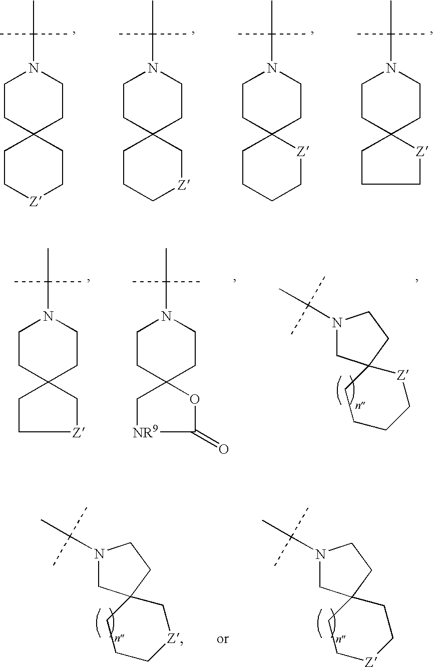

wherein n″ is 0-2 and Z′ is —NH—, —O—, or —SO₂— and the rings are optionally substituted with R^{g}, R^{h} and Rⁱ as defined in the Summary of the Invention. Preferably, R^{g} and R^{h} are hydrogen and Rⁱ is hydrogen, alkyl, cycloalkyl, or cycloalkylalkyl.

Within the above groups A, B, C, D, A(i-viii), (i-viii), and B(i-viii) and groups contained therein a particularly preferred group is that wherein R¹ is phenyl substituted with R^{a}, R^{b}, or R^{c} independently selected from hydrogen, alkyl, or halo, preferably methyl, chloro or fluoro, more preferably R¹ is 4-methylphenyl, 2,3-dichlorophenyl, 3,4-dichlorophenyl, and 2,5-dimethyl-4-chlorophenyl.

Within the above groups A, B, C, D, A(i-viii), (i-viii), and B(i-viii) and groups contained therein a particularly preferred group is that wherein R¹ is heteroaryl substituted with R^{a}, R^{b}, or R^{c} independently selected from hydrogen, alkyl, or halo.

Within the above groups A, B, C, D, A(i-viii), (i-viii), and B(i-viii) and groups contained therein a particularly preferred group is that wherein R is hydrogen and R¹ is phenyl substituted with R^{a}, R^{b}, or R^{c} independently selected from hydrogen, alkyl, or halo, preferably methyl, chloro or fluoro, more preferably R′ is 4-methylphenyl, 2,3-dichlorophenyl, 3,4-dichlorophenyl, and 2,5-dimethyl-4-chlorophenyl.

Within the above groups A, B, C, D, A(i-viii), (i-viii), and B(i-viii) and groups contained therein a particularly preferred group is that wherein R is hydrogen and R¹ is heteroaryl substituted with R^{a}, R^{b}, or R^{c} independently selected from hydrogen, alkyl, or halo.

E. In another embodiment, the invention is directed to compounds of Formula I where: R and R² are hydrogen, R³ is a group of formula (a) wherein R⁴ and R⁷ are attached to the same carbon atom and are combined together with the carbon atom to which they are attached to form a saturated or unsaturated monocyclic (C₃-C₈)cycloalkyl optionally substituted with R^{d}, R^{e}, and R^{f} as defined in the Summary of the Invention; or a saturated or unsaturated monocyclic heterocyclyl ring containing three to six ring atom wherein one or two ring atoms are selected from —(O)—, —C(—NOH), —O—, —S—, —SO—, —SO₂—, or —NH— and wherein the heterocyclic ring is substituted with R^{m}, Rⁿ and R^{o} where R^{m} and Rⁿ are independently selected from hydrogen, alkyl, hydroxyl, alkoxy, halo, haloalkyl, haloalkoxy, carboxy, or alkoxycarbonyl and R^{o} is selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy and where the aryl, heteroaryl, or heterocyclyl ring in R^{o} is optionally substituted with R^{d}, R^{e}, and R^{f} as defined in the Summary of the Invention; or

R² and R³ together with the nitrogen atom to which they are attached form spiro heterocycloamino substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, Or heterocyclyl ring in R^{g}, R^{h}, and Rⁱ is optionally substituted with R^{j}, R^{k}, or R^{l} independently selected from alkyl, halo, alkoxy, haloalkyl, hydroxyl, carboxy, alkoxycarbonyl, haloalkoxy, or cyano.

Within this embodiment, E, one group of compounds is that wherein R¹ is phenyl substituted with R^{a}, R^{b}, or R^{c} independently selected from hydrogen, alkyl, or halo, preferably methyl, chloro or fluoro, more preferably R¹ is 4-methylphenyl, 2,3-dichlorophenyl, 3,4-dichlorophenyl, and 2,5-dimethyl-4-chlorophenyl.

Within this embodiment, E another group of compounds is that wherein R′ is heteroaryl substituted with R^{a}, R^{b}, or R^{c} independently selected from hydrogen, alkyl, or halo, preferably methyl, chloro or fluoro.

F. In another embodiment, the invention is directed to compounds of Formula I where R and R² are hydrogen, R¹ is 4-methylphenyl, 2,3-dichlorophenyl, 3,4-dichlorophenyl, and 2,5-dimethyl-4-chlorophenyl, and R³ is 3,3-dimethylpiperidin-4-yl, 1-benzyl-3,3-dimethylpiperidin-4-yl, 1-methyl-3,3-dimethylpiperidin-4-yl, 1-(isobutyl)-3,3-dimethylpiperidin-4-yl, 1-(pyridin-2-ylmethyl)-3,3-dimethylpiperidin-4-yl, 1-(2-propyl)-3,3-dimethylpiperidin-4-yl, 1-(pyridin-4-ylmethyl)-3,3-dimethylpiperidin-4-yl, 1-(benzyl)-3,3-dimethylpiperidin-4-yl, 1-(acetyl)-3,3-dimethylpiperidin-4-yl, 1-(tert-butyoxycarbonyl)-3,3-dimethyl-piperidin-4-yl, 1-cyclopropyl-3,3-dimethylpiperidin-4-yl, 1-(pyridin-3-ylmethyl)-3,3-dimethylpiperidin-4-yl, 1-(2-methoxyethyl)-3,3-dimethylpiperidin-4-yl, 3,3-dimethyl-1-(2,2,2-trifluoroethyl)piperidin-4-yl, 1-ethyl-3,3-dimethylpiperidin-4-yl, 1-n-propyl-3,3-dimethylpiperidin-4-yl, 1-2,2-dimethylpropyl-3,3-dimethylpiperidin-4-yl, 1-cyclobutyl-3,3-dimethylpiperidin-4-yl, or 1-cyclopentyl-3,3-dimethylpiperidin-4-yl wherein the stereochemistry at the carbon atom at the 4-position of the piperidin-4-yl ring is (R), (S), or (RS), preferably (R) or (S).

### General Synthetic Procedures

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1994); Rodd's Chemistry of Carbon Compounds, Volumes 1-5 and Supplementals (Elsevier Science Publishers, 1989); Organic Reactions, Volumes 1-40 (John Wiley and Sons, 2003), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) can be prepared as shown in Schemes A and B below.

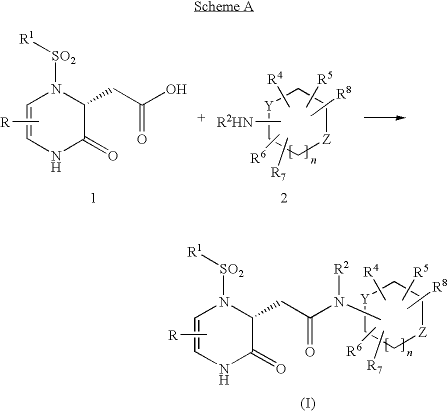

Reaction of an acid of formula 1 with an amine of formula 2 where n, Z, R, and R¹-R⁸ are as defined in the Summary of the invention under standard peptidic coupling reaction conditions provide a compound of Formula (I). The reaction is carried out in the presence of a coupling agent such as are coupled with the substituted amine 2 using standard peptide coupling conditions coupling agent (e.g., benzotriazol-1-yloxy-trispyrrolidinophosphonium hexafluorophosphate (PyBOP®.), 1-(3-dimethylamino-propyl)-3-ethylcarbodiimide hydrochloride (EDCI), O-(7-azabenzotrizol-1-yl)-1,1,3,3, tetra-methyluronium-hexafluoro-phosphate (HATU), O-benzotriazol-1-yl-N,N,N′,N′-tetramethyl-uronium hexafluorophosphate (HBTU), 1,3-dicyclohexylcarbodiimide (DCC), or the like) and optionally an appropriate catalyst (e.g., 1-hydroxybenzotriazole (HOBt), 1-hydroxy-7-azabenzotriazole (HOAt), or the like) and non-nucleophilic base (e.g., triethylamine, N-methylmorpholine, and the like, or any suitable combination thereof) at ambient temperature. Suitable reaction solvents include, but are not limited to, dimethylformamide, methylene chloride, and the like.

Amines of formula 2 are commercially available or may be prepared by methods well known in the art. For example, (R)-2,2-dimethylcyclohexanamine can be prepared according to the literature procedure (Moss, Neil; Gauthier, Jean; Ferland, Jean-Marie. Synlett (1995), 2, 142-4) from 2,2-dimethylcyclohexanone and R-(+)-alpha-Phenylethylamine. Similarly, (S) 2,2-dimethylcyclohexanamine can be prepared from S-(+)-alpha-phenylethylamine. Detailed descriptions of syntheses of amines is provided in working examples below. A few representative examples of commercially available amines are: 2-methylcyclohexanamine, 1-phenylpiperidin-4-amine, 1-benzylpiperidin-4-amine, tert-butyl 4-aminoazepane-1-carboxylate, tert-butyl 4-aminocyclohexylcarbamate, 2,7-diaza-spiro[4.4]nonane-2-carboxylic acid tert-butyl ester, 3,9-diazaspiro[5.5]undecane-3-carboxylic acid t-butyl ester, (R)-tert-butyl pyrrolidin-3-ylcarbamate, and (S)-tert-butyl pyrrolidin-3-ylcarbamate.

Compounds of formula 1 where R is hydrogen can be prepared as described in Scheme B below.

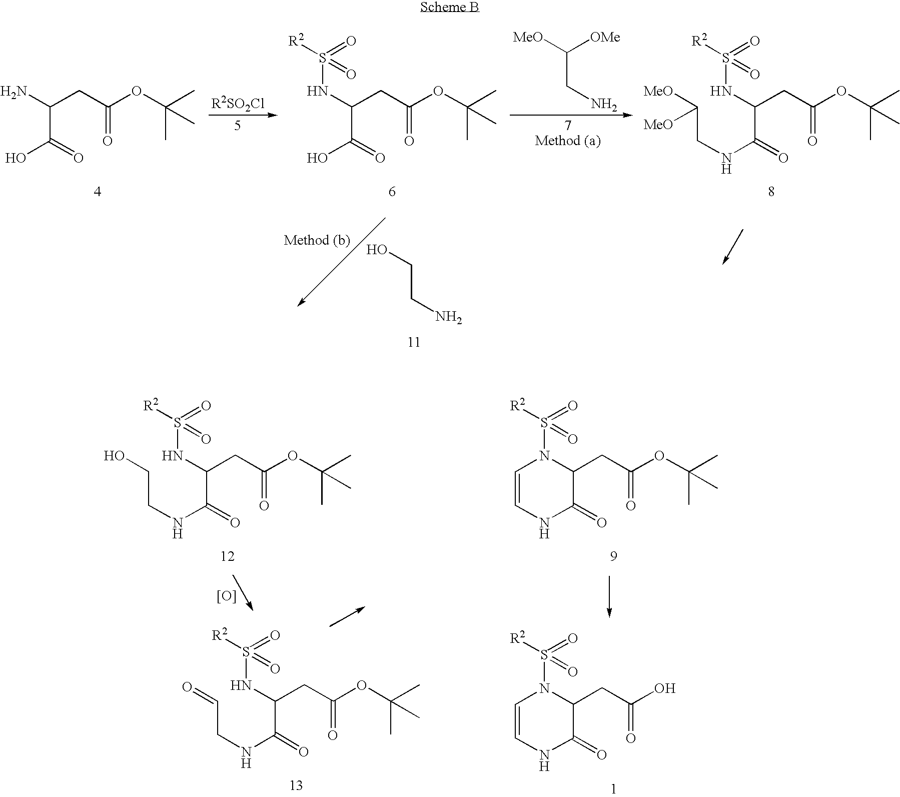

Sulfonylation of 2-amino-4-tert-butoxy-4-oxobutanoic acid with a sulfonyl chloride of formula 5 provides a compound of formula 6. The reaction is carried out in the presence of a base, preferably an inorganic base such as sodium carbonate and a suitable organic solvent optionally in the presence of a base such as dioxane and water. Compound 4 such as (R)-2-amino-4-tert-butoxy-4-oxobutanoic acid is commercially available from a number of vendors including Chem-Impex international, Inc.) or prepared it can be prepared according to literature procedures such as Schabbert, S.; Pierschbacher, M. D.; Mattern, R.; Goodman, M. Bioorg. Med. Chem. 2002, 10, 3331-7; and Bold, G.; Duthaler, R. O.; Riediker, M. Angew. Chem. 1989, 101, 491-3. Compounds of Formula 5 are commercially available or can be prepared by methods well known in the art. Compounds of formula 1 where R is alkyl can be prepared by utilizing compounds of formula 4 substituted with alkyl group.

Compound 6 can be converted to a compound of formula 1 via Method (a) or (b) shown above. In method (a), compound 6 is reacted with the amine 7 under coupling reaction conditions described above to afford a compound of formula 8. Compound 8 is cyclized to compound 9 in the presence of a catalytic amount of an acid such as TsOH under elevated temperature such as at 60° C. Hydrolysis of the ester group in 10 under acidic hydrolysis reaction conditions such as TFA in DCM yields the acid 10.

In Method (b), compound 6 is coupled with the amino alcohol 11 under coupling reaction conditions described above to afford a compound of formula 12. Alcohol 12 is then oxidized to the corresponding carbonyl compound 13 with an oxidizing agent such as Dess-Martin periodinane (see J. Org. Chem. 1983, 48, 4155), and the like. The carbonyl compound 13 is then cyclized to compound 1 under acidic conditions described above.

Compound of Formula (I) can be converted to other compounds of Formula (I). For example, the free amino group in compound of Formula (I) can be alkylated with electrophiles (e.g. alkyl halids) or aldehydes (through reductive aminations). Examples of these transformations are illustrated in the experimental section.

Salts of a compound of Formula I with a salt-forming group may be prepared in a manner known per se. Acid addition salts of compounds of Formula I may thus be obtained by treatment with an acid or with a suitable anion exchange reagent. A salt with two acid molecules (for example a dihalogenide of a compound of Formula I) may also be converted into a salt with one acid molecule per compound (for example a monohalogenide); this may be done by heating to a melt, or for example by heating as a solid under a high vacuum at elevated temperature, for example from 130-170° C., one molecule of the acid being expelled per molecule of a compound of Formula I.

Salts can usually be converted to free compounds, e.g. by treating with suitable basic agents, for example with alkali metal carbonates, alkali metal hydrogen carbonates, or alkali metal hydroxides, typically potassium carbonate or sodium hydroxide.

### Utility

The compound of Formula (I) are B1 receptor antagonists and hence are useful in the treatment of a disorder such as acute pain, dental pain, back pain, lower back pain, pain from trauma, surgical pain, pain resulting from amputation or abscess, causalgia, fibromyalgia, demyelinating diseases, trigeminal neuralgia, cancer, chronic alcoholism, stroke, thalamic pain syndrome, diabetes, acquired immune deficiency syndrome (“AIDS”), toxins and chemotherapy, general headache, migraine, cluster headache, mixed-vascular and non-vascular syndromes, tension headache, general inflammation, arthritis, rheumatic diseases, lupus, osteoarthritis, inflammatory bowel disorders, inflammatory eye disorders, inflammatory or unstable bladder disorders, psoriasis, skin complaints with inflammatory components, sunburn, carditis, dermatitis, myositis, neuritis, collagen vascular diseases, chronic inflammatory conditions, inflammatory pain and associated hyperalgesia and allodynia, neuropathic pain and associated hyperalgesia and allodynia, diabetic neuropathy pain, sympathetically maintained pain, deafferentation syndromes, asthma, vasomotor or allergic rhinitis, epithelial tissue damage or dysfunction, herpes simplex, post-herpetic neuralgia, disturbances of visceral motility at respiratory, genitourinary, gastrointestinal or vascular regions, wounds, burns, allergic skin reactions, pruritis, vitiligo, general gastrointestinal disorders, colitis, inflammatory bowel disease, gastric ulceration, duodenal ulcers, thalamic pain syndrome, diabetes, toxins and chemotherapy, septic shock, and bronchial disorders.

### Biological Testing

The in vitro binding affinity of the compounds of the invention to the human B1 and B2 bradykinin receptors can be tested using the radioligand binding assay described in Biological Example 1 below. The antagonistic activity of the compounds of the invention for the human B1 and B2 bradykinin receptors can be tested using the calcium flux assay, Rabbit endotbelial cell B1-specific PGI₂ secretion Assay, and umbilical vein Assay described in Biological Examples 2 and 3 below. The antinociceptive activity of the compounds of the invention was determined using the rat and monkey pain models described in Example 4 below. The anti-inflammatory activity of the compounds of the invention was determined using the Green Monkey LPS inflammation model described in Example 5 below.

### Pharmaceutical Compositions and Administration

Also embraced within this invention is a class of pharmaceutical compositions comprising the active compounds of the invention in association with one or more non-toxic, pharmaceutically-acceptable carriers and/or diluents and/or adjuvants (collectively referred to herein as “carrier” materials) and, if desired, other active ingredients. The active compounds of the present invention can be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, preferably from about 1 to 500 mg or 5 to 1000 mg. A suitable daily dose for a human or other mammal may vary widely depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, preferably between about 0.1 and about 50 mg/kg, and more preferably about 0.1 and about 20 mg/kg body weight may be appropriate. The daily dose can be administered in one to four doses per day.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In the case of psoriasis and other skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, or pastes) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include DMSO and related analogs.

The compounds of this invention can also be administered by a transdermal device. Preferably transdermal administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (i.e., propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

While the compounds of the invention can be administered as the sole active pharmaceutical agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

The present compounds may also be used in combination therapies with opioids and other anti-pain analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists, COX-2 inhibitors such as celecoxib, rofecoxib, valdecoxib, parecoxib, and darecoxib, NSAID's, and sodium channel blockers, among others. More preferred would be combinations with compounds selected from morphine, meperidine, codeine, pentazocine, buprenorphine, butorphanol, dezocine, meptazinol, hydrocodone, oxycodone, methadone, tetrahydrocannibinol, pregabalin, Tramadol [(+) enantiomer], DuP 747, Dynorphine A, Enadoline, RP-60180, HN-11608, E-2078, ICI-204448, acetominophen (paracetamol), propoxyphene, nalbuphine, E-4018, filenadol, mirtentanil, amitriptyline, DuP631, Tramadol [(−) enantiomer], GP-531, acadesine, AKI-1, AKI-2, GP-1683, GP-3269, 4030W92, tramadol racemate, Dynorphine A, E-2078, AXC3742, SNX-111, ADL2-1294, ICI-204448, CT-3, CP-99,994, and CP-99,994.

Alternatively, the present compounds may also be used in co-therapies with other treatments for inflammation, e.g. steroids, NSAIDs, iNOS inhibitors, p38 inhibitors, TNF inhibitors, 5-lipoxygenase inhibitors, LTB₄ receptor antagonists and LTA4 hydrolase inhibitors.

### EXAMPLES

In order that the invention described herein may be more readily understood, the following preparations of compounds of Formula (I) and intermediates (References) are set forth. It should be understood that these examples are for illustrative purposes only and are not to be construed as limiting this invention in any manner. Unless otherwise noted, all materials were obtained from commercial suppliers and used without further purification. All parts are by weight unless otherwise indicated. All compounds showed NMR spectra consistent with their assigned structures. Melting points were determined on a Buchi apparatus and are uncorrected. Mass spectral data was determined by electrospray ionization technique. All examples were purified to >90% purity as determined by high-performance liquid chromatography.

### Synthetic Examples

### Reference 1

### Synthesis of (R)-2-(3-oxo-1-tosyl-1,2,3,4-tetrahydropyrazin-2-yl)acetic acid

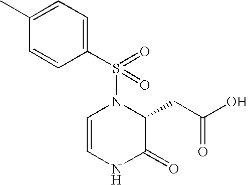

### Step 1

To (D)-Aspartic acid β-tert-butyl ester (Chem-impex, 29.5 g, 0.142 mol) in dioxane (500 mL) and water (500 mL) at room temperature was added sodium carbonate (38.7 g) followed by p-toluenesulfonyl chloride (28 g, 0.146 mol) portion wise. The reaction mixture was stirred overnight and then carefully acidified with 10% HCl and brine. The mixture was extracted with EtOAc. The combined organic layer was washed with brine, dried, and evaporated to give (R)-4-tert-butoxy-2-(4-methylphenylsulfonamido)-4-oxobutanoic acid.

### Step 2

(R)-4-tert-Butoxy-2-(4-methylphenylsulfonamido)-4-oxobutanoic acid (60 g, 0.124 mol) was dissolved in anhydrous DME (400 mL) and HOBt (Aldrich, 19.2 g) and aminoacetaldehyde dimethyl acetal (Aldrich, 16 mL) were added followed by EDCI (Aldrich, 30 g). The reaction mixture was stirred at room temperature overnight. EtOAc (1000 mL) and water (1000 mL) were added. The EtOAc layer was washed with brine, diluted with HCl/brine, and 10% sodium carbonate/brine, dried, and evaporated to give (R)-tert-butyl 4-(2,2-dimethoxyethylamino)-3-(4-methylphenylsulfonamido)-4-oxobutanoate.

### Step 3

(R)-tert-Butyl 4-(2,2-dimethoxyethylamino)-3-(4-methylphenylsulfonamido)-4-oxobutanoate (54 g) was dissolved in anhydrous dioxane (IL) and p-toluenesulfonic acid (6.6 g) was added. The reaction mixture was heated at 60° C. for 17 h until LC/MS indicated that the starting material was consumed. The reaction mixture was cooled to RT and concentrated to about 100 mL. EtOAc (IL) was added and the solution was washed with sodium bicarbonate solution/brine, dried and evaporated. Column chromatograph (20-50% EtAOAc/hexanes, silica gel) gave (R)-tert-butyl 2-(3-oxo-1-tosyl-1,2,3,4-tetrahydropyrazin-2-yl)acetate.

### Step 4

(R)-tert-Butyl 2-(3-oxo-1-tosyl-1,2,3,4-tetrahydropyrazin-2-yl)acetate (33.5 g) was dissolved in DCM (400 mL) and TFA (160 mL) was added. The reaction mixture was stirred at RT until TLC (50% EtOAc/hexanes) indicated the reaction was complete. The reaction mixture was evaporated. DCM (200,mL) was added and the solution was evaporated again. The resulting residue was stirred with ether (500 mL) for 1 h. The solids were filtered and washed with ether to give (R)-2-(3-oxo-1-tosyl-1,2,3,4-tetrahydropyrazin-2-yl)acetic acid.

Following the procedure described above, the following compounds were prepared:
(R)-2-(3-oxo-1-(phenylsulfonyl)-1,2,3,4-tetrahydropyrazin-2-yl)acetic acid;
(R)-2-(1-(2-methylphenylsulfonyl)-3-oxo-1,2,3,4-tetrahydropyrazin-2-yl)acetic acid;
(R)-2-(1-(3-methylphenylsulfonyl)-3-oxo-1,2,3,4-tetrahydropyrazin-2-yl)acetic acid;
(R)-2-(1-(3-chlorophenylsulfonyl)-3-oxo-1,2,3,4-tetrahydropyrazin-2-yl)acetic acid;
(R)-2-(1-(4-chlorophenylsulfonyl)-3-oxo-1,2,3,4-tetrahydropyrazin-2-yl)acetic acid;
(R)-2-(1-(4-methoxyphenylsulfonyl)-3-oxo-1,2,3,4-tetrahydropyrazin-2-yl)acetic acid;
(R)-2-(1-(3-trifluorophenylsulfonyl)-3-oxo-1,2,3,4-tetrahydropyrazin-2-yl)acetic acid;
(R)-2-(1-(3,4-dichlorophenylsulfonyl)-3-oxo-1,2,3,4-tetrahydropyrazin-2-yl)acetic acid;
(R)-2-(1-(2,3-dichlorophenylsulfonyl)-3-oxo-1,2,3,4-tetrahydropyrazin-2-yl)acetic acid;
(R)-2-(1-(4-chloro-2,5-dimethylphenylsulfonyl)-3-oxo-1,2,3,4-tetrahydropyrazin-2-yl)acetic acid; and
(R)-2-(1-(cyclopropylsulfonyl)-3-oxo-1,2,3,4-tetrahydropyrazin-2-yl)acetic acid.

### Reference 2

### Synthesis of (R)-2-(5-methyl-3-oxo-1-tosyl-1,2,3,4-tetrahydropyrazin-2-yl)acetic acid

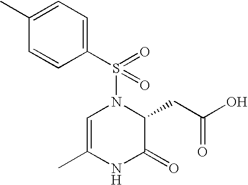

### Step 1

(3R)-tert-Butyl 4-(1-hydroxypropan-2-ylamino)-3-(4-methylphenylsulfonamido)-4-oxobutanoate was prepared from (R)-4-tert-butoxy-2-(4-methylphenylsulfonamido)-4-oxobutanoic acid and 2-aminopropan-1-ol according to Example 1, Step 1.

### Step 2

To (3R)-tert-butyl 4-(1-hydroxypropan-2-ylamino)-3-(4-methylphenylsulfonamido)-4-oxobutanoate (1.5 g, 3.7 mmol) in wet dichloromethane (in a separatory funnel dichloromethane was shaken with water, and separated) added Dess-Martin periodinane (Aldrich, 3.2 g, 7.5 mmol) and the reaction mixture was stirred for one h. Additional 9 ml of wet DCM was added and stirring was continued for an additional 1 h. The reaction mixture was then concentrated. EtOAc was added and the mixture was washed with 10% Na₂S₂O₃ and sat NaHCO₃ (20 mL), brine, dried, and evaporated to give crude (3R)-tert-butyl 3-(4-methylphenylsulfonamido)-4-oxo-4-(1-oxopropan-2-ylamino)butanoate.

### Step 3

To (3R)-tert-butyl 3-(4-methylphenylsulfonamido)-4-oxo-4-(1-oxopropan-2-ylamino)butanoate (1.1 g, 3 mmol) in dioxane was added p-toluenesulfonic acid monohydrate (0.3 g, 2 mmol) and the reaction mixture was heated at 60° C. for 23 h and then concentrated. EtOAc was added and the mixture was washed with NaHCO₃/brine, brine, dried and evaporated. Column chromatograph purification (silica gel, 20-33% EtOAc/hexanes) of the residue gave (R)-tert-butyl 2-(5-methyl-3-oxo-1-tosyl-1,2,3,4-tetrahydropyrazin-2-yl)acetate.

### Step 4

To (R)-tert-butyl 2-(5-methyl-3-oxo-1-tosyl-1,2,3,4-tetrahydropyrazin-2-yl)acetate (0.27 g, 0.71 mmol) in DCM (10 mL) was added TFA (5 mL). The reaction mixture was stirred for 3 h and concentrated and the residue was then co-evaporated with DCM. The residue was trituated with ether to give the title compound. MS: 323 (M−1).

### Reference 3

### Synthesis of trans-4-methoxycyclohexylamine

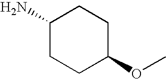

### Step 1

To a 100-mL round-bottomed flask containing (1R,4R)-4-aminocyclohexanol hydrochloride (4.00 g, 26.4 mmol, Aldrich), was added di-tert-butyldicarbonate (5.76 g, 26.4 mmol, Aldrich) and sodium carbonate (3.91 g, 36.9 mmol, J. T. Baker) in THF/H₂O (20 mL/20 mL). The reaction mixture was stirred at RT for 10 h. The product was extracted with EtOAc (20 mL), and the organic phase was washed with 5% brine (15 mL), and dried over Na₂SO₄, filtered, and concentrated in vacuo to yield tert-butyl (1R,4R)-4-hydroxycyclohexylcarbamate.

### Step 2

To a 100-mL round-bottomed flask containing tert-butyl (1R,4R)-4-hydroxy-cyclohexylcarbamate (0.500 g, 2.32 mmol), was added silver dioxide (0.753 g, 3.25 mmol, Aldrich) and iodomethane (0.659 g, 4.64 mmol, Aldrich) in MeCN (10 mL). The reaction mixture was stirred at 120° C. under N₂ for 2.5 h. The reaction mixture was quenched with 5% brine (10 mL), and the product was extracted with EtOAc (15 mL), and the organic phase was washed with 5% brine (15 mL), dried over Na₂SO₄, filtered, and concentrated to yield tert-butyl (1R,4R)-4-methoxycyclohexylcarbamate.

### Step 3

To a 100-mL round-bottomed flask containing tert-butyl (1R,4R)-4-methoxy-cyclohexylcarbamate (0.50 g, 2.2 mmol) was added hydrochloride acid (11 ml, 44 mmol, 4 M, Aldrich) in 1,4-dioxane (Aldrich). The reaction mixture was stirred at RT for 1 h. Evaporation of the solvent gave (1R,4R)-4-methoxycyclohexylamine hydrochloride salt.

### Reference 4

### Synthesis of tert-butyl 4-amino-3,3-dimethylpiperidine-1-carboxylate

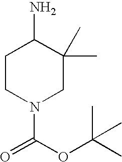

### Step 1

To a solution of tert-butyl 4-oxopiperidine-1-carboxylate (10.12 g, 50.8 mmol) in dry THF at 0° C. was added NaH (2.56 g, 107 mmol, 60% in mineral oil, 4.27 g) and then iodomethane (7.91 ml, 127 mmol). The reaction mixture was stirred at RT for 48 h. The solvent was evaporated and the residue was extracted with ether, washed with water and brine. The organic phase was concentrated, treated with small amount of hexanes to precipitate out tert-butyl 3,3-dimethyl-4-oxopiperidine-1-carboxylate.

### Step 2

To a mixture of tert-butyl 3,3-dimethyl-4-oxopiperidine-1-carboxylate (2.98 g, 13.1 mmol) and benzylamine (5.73 ml, 52.4 mmol) in 10 mL of 1,2-dichloroethane was added titanium (4+) isopropoxide (4.60 ml, 15.7 mmol) and the resulting mixture was stirred at RT for 12 h. The mixture was diluted with MeOH (15 mL) and sodium borohydride (1.39 ml, 39.3 mmol) was added. After stirring at RT for 20 min, the reaction mixture was quenched with 5 mL of sat. NH₄Cl and the solvent was evaporated to dryness. The residue was flash chromatographed (SiO₂, EtOAc/Hexane=1:1 to 2:1 to pure EtOAc) to give tert-butyl 4-(benzylamino)-3,3-dimethylpiperidine-1-carboxylate as a sticky oil which was used directly in the next step.

### Step 3

A suspension of tert-butyl 4-(benzylamino)-3,3-dimethylpiperidine-1-carboxylate (2.5 g, 8 mmol) and palladium, 10 wt. % on activated carbon (250 mg) in EtOAc (80 mL) was degassed and purged with hydrogen. After stirring under hydrogen atmosphere for 48 h, the reaction mixture was filtered through a silica gel pad with the help of EtOAc/2M NH₃ in MeOH=100:30 to give tert-butyl 4-amino-3,3-dimethylpiperidine-1-carboxylate.

Similarly 3,3-dimethyltetrahydro-2H-pyran-4-amine (from tetrahydropyran-4-one) was prepared.

### Reference 5

### Synthesis of tert-butyl 3,3-dimethyl-4-(methylamino)piperidine-1-carboxylate

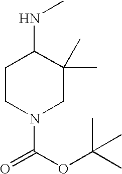

To a solution of methanamine (14.3 mL of 2M in THF) and tert-butyl 3,3-dimethyl-4-oxopiperidine-1-carboxylate (1.30 g, 5.719 mmol) in dichloroethane was added acetic acid, 99.5% (4.954 ml, 85.790 mmol) and sodium triacetoxyborohydride (2.424 g, 11.439 mmol) and the resulting solution was stirred at RT overnight. After stirring at RT for 48 h, the solvent was evaporated to dryness and the reaction mixture was diluted with EtOAc, quenched with Sat. NaHCO₃, extracted with EtOAc, dried over Na₂SO₄ and evaporated to dryness. Column chromatography (SiO2,EtOAc to EtOAc/2M NH₃ in MeOH=100:10 to 100:20) gave tert-butyl 3,3-dimethyl-4-(methylamino)piperidine-1-carboxylate.

### Reference 6

### Synthesis of tert-butyl 4-(ethylamino)piperidine-1-carboxylate

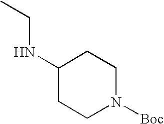

To a solution of ethanamine (30.0 ml, 60.0 mmol, 2M solution in THF) and tert-butyl 4-oxopiperidine-1-carboxylate (30.0 ml, 60.0 mmol) in THF (30.0 ml, 15.1 mmol) was added sodium triacetoxyborohydride (6.4612 g, 30.5 mmol), and stirred at r.t. under N₂. After 1 h, acetic acid (2.00 ml, 34.9 mmol) was added and the reaction mixture was stirred overnight before diluting with AcOEt (200 ml) and sat'd NaHCO₃ aq. (100 ml). The aqueous layer was extracted with AcOEt and the combined organic layer was washed with sat'd NaHCO₃ aq. (100 ml) and sat'd NaCl (100 ml), and dried over Na₂SO₄. Major portion of the product was found in the aqueous layer, so it was extracted with CH₂Cl 2 and dried over Na₂SO₄. The solvent was removed under reduced pressure and chromatographed on silica (CH₂Cl₂→CH₂Cl₂/MeOH+2N NH₃=10/1) to yield the desired product as a colorless liquid.

### Reference 7

### Synthesis of 1-tert-butyl 4(R/S)-aminoazepane-1-carboxylate

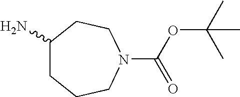

### Step 1

To a solution of tert-butyl 4-oxoazepane-1-carboxylate (2 g, 9 mmol) and benzylamine (4.00 ml, 37 mmol) in tetrahydrofuran (15.0 ml, 9 mmol) and was added acetic acid (1.00 ml, 17 mmol) followed by sodium triacetoxyborohydride (4.018 g, 19 mmol. After 18 h, H₂O (20 ml) was added. The solution was partitioned into CH₂Cl₂ (200 ml) and saturated NaHCO₃ (100 ml), and the aqueous layer was extracted with CH₂Cl₂. The combined organic layer was washed with saturated NaHCO₃ (100 ml) and saturated NaCl (100 ml), and dried over Na₂SO₄. The solvent was removed under reduced pressure and chromatographed on silical (ISCO(40 g): CH₂Cl₂ to CH₂Cl₂/MeOH+2NNH₃=10/1) to yield tert-butyl 4-(benzylamino)azepane-1-carboxylate.

### Step 2

tert-Butyl 4-(benzylamino)azepane-1-carboxylate (2.3591 g) was dissolved in ethyl acetate (50.0 ml) and 10% Pd/C (0.94 g). The reaction mixture was stirred under hydrogen atmosphere. After 36 h, the reaction mixture was filtered through Celite, the catalyst was washed with ethyl acetate, and concentrated under reduced pressure to yield the title compound.

### Reference 8

### Synthesis of tert-butyl 4 (R/S)-(ethylamino)azepane-1-carboxylate

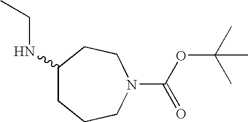

### Step 1

To a solution of ethanamine (10.0 ml, 20 mmol) and tert-butyl 4-oxoazepane-1-carboxylate (1 g, 5 mmol) in THF (10.0 ml, 5 mmol) and acetic acid (0.650 ml, 11 mmol) was added sodium triacetoxyborohydride (˜2.4 g, 11 mmol). After 24 h, the reaction mixture was diluted into CH₂Cl₂ (100 ml) and sat'd NaHCO₃ aq. (50 ml) [11:50], and the organic layer was extracted with CH₂Cl₂. The combined organic phase was washed with sat'd NaHCO₃ aq. (50 ml) and sat'd NaCl aq. (50 ml), dried over Na₂SO₄. The solvent was removed under reduced pressure and chromatographed on silica (ISCO(12 g): CH₂Cl₂->CH₂Cl₂/MeOH+2NNH₃=10/1) to yield the title compound:

### Reference 9

### Synthesis of N,N-dimethylazepan-4(R/S)-amine dihydrochloride

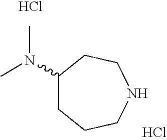

### Step 1

To a solution of tert-butyl 4-aminoazepane-1-carboxylate (0.3042 g, 1.42 mmol), formaldehyde (37w % in water) (1.50 ml, 20.1 mmol) in THF (15.0 ml, 1.42 mmol) was added sodium triacetoxy borohydride (0.6117 g, 2.89 mmol). After 23 h, the reaction mixture was evaporated and the residue was partitioned into CH₂Cl₂ (50 ml) and sat'd NaHCO₃ aq. (50 ml). The organic phase was collected and the aqueous layer was extracted with CH₂Cl₂ (50 ml×2) and the combined organic layer was washed with sat'd NaCl aq. (50 ml), dried over Na₂SO₄. The solvent was removed under reduced pressure and dried in vacuo to yield tert-butyl 4-(R/S)-(dimethylamino)azepane-1-carboxylate.

### Step 2

To a solution of tert-butyl 4-(dimethylamino)azepane-1′-carboxylate (0.3032 g, 1.25 mmol) in 1,4-dioxane (2.00 ml, 23.4 mmol) was added hydrogen chloride (4M in 1,4-dioxane) (10.0 ml, 40.0 mmol). After 24 h, methanol (2.00 ml, 1.25 mmol) was added and evaporated. The residue was treated with Et₂O and sonicated and triturated to give the title compound.

### Reference 10

### Synthesis of cyclooctane-1,5-diamine

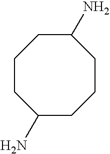

A mixture of N1,N5-dibenzylcyclooctane-1,5-diamine (0.350 g, 1.09 mmol, prepared from cyclooctane-1,5-dione and benzyl amine according to procedures similar to Reference 7) and 10% Pd/C (0.0231 g, 0.217 mmol), and acetic acid (0.0130 g, 0.217 mmol) in EtOAc/EtOH was stirred under H₂ atmosphere for 14 h. Filtration of the reaction mixture through the celite, followed by removal of the solvent gave the title compound.

### Reference 11

### Synthesis of tert-butyl 5-aminoazocane-1-carboxylate and tert-butyl 4-aminoazocane-1-carboxylate

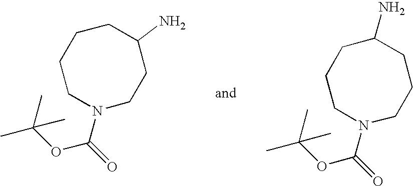

### Step 1

To a solution of tert-butyl 4-oxoazepane-1-carboxylate (6.47 g, 30 mmol) in THF (20 ml), cooled to −35° C. with a cooling bath (dry ice/isopropanol), was added simultaneously with a solution of ethyl diazoacetic acid (4.1 ml, 39 mmol) in 5 ml of THF and BF₃ ether (3.8 ml, 30 mmol) in 5 ml of THF over a period of 15 min. After the addition, the reaction mixture was stirred at the temperature for additional 1 h, and then slowly warmed to RT and stirred at RT for 1.5 h. The reaction mixture was quenched with sat. Na₂CO₃, and the product was extracted with EtOAc, washed with water, dried over Na₂SO₄, filtered, and concentrated to give the crude product. Silica gel chromatography with 3:1 hexane/EtOAc gave 1-tert-butyl 5-ethyl 4-oxoazocane-1,5-dicarboxylate and 1-tert-butyl 4-ethyl 5-oxoazocane-1,4-dicarboxylate.

### Step 2

A mixture of 1-tert-butyl 4-ethyl 5-oxoazocane-1,4-dicarboxylate (2.50 g, 8.35 mmol) and lithium hydroxide hydrate (1.40 g, 33.4 mmol) in MeOH/H₂O (5 ml/5 ml) was refluxed at 80° C. for 2 h. The solvent was evaporated and the product was extracted with EtOAc, dried over Na₂SO₄, concentrated to give tert-butyl 5-oxoazocane-1-carboxylate.

Similarly tert-butyl 4-oxoazocane-1-carboxylate was prepared from 1-tert-butyl 4-ethyl 5-oxoazocane-1,4-dicarboxylate. Both tert-butyl 5-oxoazocane-1-carboxylate and tert-butyl 4-oxoazocane-1-carboxylate were converted to the corresponding amines, tert-butyl 5-aminoazocane-1-carboxylate and tert-butyl 4-aminoazocane-1-carboxylate, according to the procedures in Reference 7.

### Reference 12

### Synthesis of (S)-tert-butyl 4-amino-3,3-dimethylpiperidine-1-carboxylate

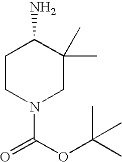

### Step 1

To a solution of tert-butyl 4-oxopiperidine-1-carboxylate (150 g) in dry THF (750 ml) at 5° C. was added sodium hydride (66 g) and the reaction mixture was warmed to 20° C. Methyl iodide (113 ml) was added between 25-35° C. in about 45 min. The reaction was stirred for additional 2 h. The solvent was evaporated and the residue extracted with ether (3×600 ml), washed with water and brine. The organic phase was concentrated and treated with hexane (350 ml) to precipitate tert-butyl 3,3-dimethyl-4-oxopiperidine-1-carboxylate.

### Step 2

Titanium tetrachloride (18 ml) was added drop wise to a vigorously stirred solution of S-phenylethylamine (48 g) and triethyl amine (275 mL) in dry CH₂Cl₂ (450 ml) under nitrogen atmosphere. tert-Butyl 3,3-dimethyl-4-oxopiperidine-1-carboxylate (75 g) dissolved in CH₂Cl₂ (225 mL) was added to the above mixture over 10 min. The reaction mixture was stirred and refluxed for 4 h. After 4 h, the reaction mixture was cooled, diluted with diethyl ether (600 ml) and filtered through celite and was concentrated in vacuo to afford the crude (S,E)-tert-butyl 3,3-dimethyl-4-(1-phenylethylimino)piperidine-1-carboxylate which was used directly in the next reaction.

### Step 3

To the above solution of the imine (105 g) in absolute ethanol (600 ml) was added sodium borohydride (6 g) at −78° C. The reaction mixture was allowed to warm up to −20° C. and then quenched with 6N HCl. Ethanol was removed and the residue was mixed with water (500 ml) and then extracted with ether. The organic layer was washed with brine, dried and concentrated to provide the crude mass. This material was chromatographed (10% EtOAc in hexane) over silica gel to obtain (S)-tert-butyl 3,3-dimethyl-4-((S)-1-phenylethylamino)-piperidine-1-carboxylate as pure liquid.

### Step 4

(S)-tert-Butyl 3,3-dimethyl-4-((S)-1-phenylethylamino)-piperidine-1-carboxylate (45 g) was dissolved in methanol (350 ml) and was stirred under hydrogen atmosphere (1 atm.). When TLC revealed no starting material was left, the reaction mixture was filtered and concentrated. The crude product was purified by column chromatography over silica gel (eluent, EtOAc, followed by 5% MeOH/EtOAc) to give (S)-tert-butyl 4-amino-3,3-dimethylpiperidine-1-carboxylate as a liquid. The product stereochemistry was assigned based on the literature (Moss, Neil; Gauthier, Jean; Ferland, Jean-Marie. Synlett 1995, (2), 142-4).

Similarly, (R)-tert-butyl 4-amino-3,3-dimethylpiperidine-1-carboxylate was prepared using (R)-phenylethylamine. (R)-3,3-dimethyl-tetrahydro-2H-pyran-4-amine and (S)-3,3-dimethyl-tetrahydro-2H-pyran-4-amine were prepared from 3,3-dimethyl-tetrahydropyran-4-one which was prepared from tetrahydropyran-4-one.

### Example 1

### Synthesis of tert-butyl 3,3-dimethyl-4-(2-((R)-3-oxo-1-tosyl-1,2,3,4-tetrahydropyrazin-2-yl)acetamido)piperidine-1-carboxylate

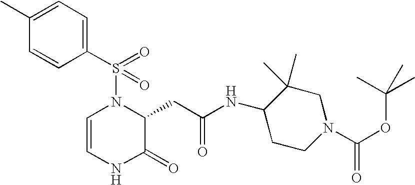

A solution of (R)-2-(3-oxo-1-tosyl-1,2,3,4-tetrahydropyrazin-2-yl)acetic acid (4.0 g, 13 mmol), tert-butyl 4-amino-3,3-dimethylpiperidine-1-carboxylate (3.50 g, 15 mmol), 1-hydroxybenzotriazole (1.7 g, 13 mmol) and N-(3-dimethylaminopropyl)-N′-ethylcarbodiimide hydrochloride (2.5 g, 13 mmol) in DMF (1.5 mL) was stirred at RT for 12 h. The reaction mixture was quenched with sat. NaHCO₃, extracted with EtOAc/hexane=2:1, washed with brine, dried over Na₂SO₄, filtered and evaporated to dryness. Column chromatograph (SiO₂, EtOAc/hexane=1:1 to 2:1 to pure EtOAc) afforded tert-butyl 3,3-dimethyl-4-(2-((R)-3-oxo-1-tosyl-1,2,3,4-tetrahydropyrazin-2-yl)acetamido)piperidine-1-carboxylate. MS 521 (M+1).

### Example 2

### Synthesis of N-(3,3-dimethylpiperidin-4-yl)-2-((R)-3-oxo-1-tosyl-1,2,3,4-tetrahydropyrazin-2-yl)acetamide

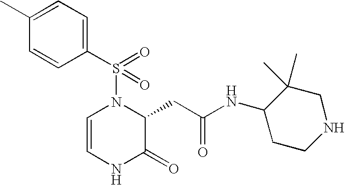

To a solution of tert-butyl 3,3-dimethyl-4-(2-((R)-3-oxo-1-tosyl-1,2,3,4-tetrahydropyrazin-2-yl)acetamido)piperidine-1-carboxylate (2.50 g, 4.8 mmol) in DCM (20 mL) was added 1N HCl (24 mL) in ether and the resulting solution was stirred overnight. The solvent was evaporated and the residue was solidified with 100 mL of ethyl ether to afford the titled product as a white solid. MS 421 (M+1).

### Example 3

### Synthesis of N-(1-acetyl-3,3-dimethylpiperidin-4-yl)-2-((R)-3-oxo-1-tosyl-1,2,3,4-tetrahydropyrazin-2-yl)acetamide

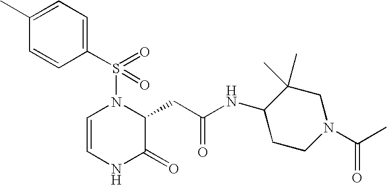

A solution or N-(3,3-dimethylpiperidin-4-yl)-2-((R)-3-oxo-1-tosyl-1,2,3,4-tetrahydropyrazin-2-yl)acetamide (42 mg, 100 μmol), acetyl anhydride (12 μl, 130 μmol) and triethylamine (28 μl, 200 μmol) in DCM was stirred for 30 min. The solvent was diluted with EtOAc, washed with brine, dried over Na₂SO₄, filtered and evaporated to dryness. Flash chromatography (SiO₂, hexane/EtOAc=1:1 to pure EtOAc to EtOAc/MeOH=100:10) gave N-(1-acetyl-3,3-dimethylpiperidin-4-yl)-2-((R)-3-oxo-1-tosyl-1,2,3,4-tetrahydropyrazin-2-yl)acetamide as a white solid. MS 463 (M+1).

### Example 4

### Synthesis of N-(3,3-dimethyl-1-(pyridin-4-ylmethyl)piperidin-4-yl)-2-((R)-3-oxo-1-tosyl-1,2,3,4-tetrahydropyrazin-2-yl)acetamide

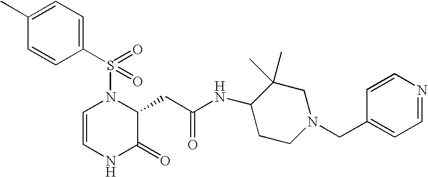

To a solution of 4-pyridinecarboxaldehyde (27 μl, 285 μmol) and N-(3,3-dimethylpiperidin-4-yl)-2-((R)-3-oxo-1-tosyl-1,2,3,4-tetrahydropyrazin-2-yl)acetamide (40 mg, 95 μmol) in dichloroethane was added sodium triacetoxyborohydride (40 mg, 190 μmol) and the resulting solution was stirred at RT overnight. The reaction was quenched with sat. NaHCO₃, extracted with EtOAc, dried over Na₂SO₄ and evaporated to dryness. Flash chromatography (SiO₂, EtOAc to EtOAc/2M NH₃ in MeOH=100:10 to 100:20) gave N-(3,3-dimethyl-1-(pyridin-4-ylmethyl)piperidin-4-yl)-2-((R)-3-oxo-1-tosyl-1,2,3,4-tetrahydropyrazin-2-yl)acetamide. MS 512 (M+1).

### Example 5

### Synthesis of N-(3,3-dimethyl-1-(2,2,2-trifluoroethyl)piperidin-4-yl)-2-((R)-3-oxo-1-tosyl-1,2,3,4-tetrahydropyrazin-2-yl)acetamide

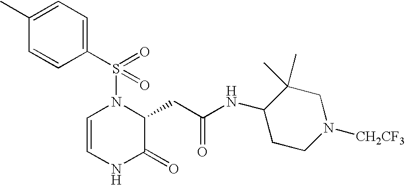

N-(3,3-dimethylpiperidin-4-yl)-2-((R)-3-oxo-1-tosyl-1,2,3,4-tetrahydropyrazin-2-yl)acetamide hydrochloride (65 mg, 142 μmol), monosodium hydrogen carbonate (17 μl, 427 μmol), and 2,2,2-trifluoroethyl trichloromethanesulphonate (60 μl, 213 μmol) were combined in CH₃CN, and the reaction mixture was refluxed overnight. The mixture was then concentrated. Water and ethyl acetate were added, and the ethyl acetate layer was separated, dried with sodium sulfate, filtered and concentrated. The mixture was then purified column chromatograph (silica gel, with 0 to 10% MeOH in DCM) to give the title product. MS 503 (M+1).

### Example 6

### Synthesis of (R)-tert-butyl 4-(N-ethyl-2-(3-oxo-1-tosyl-1,2,3,4-tetrahydropyrazin-2-yl)acetamido)piperidine-1-carboxylate

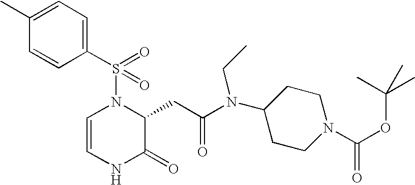

To a solution of (R)-2-(3-oxo-1-tosyl-1,2,3,4-tetrahydropyrazin-2-yl)acetic acid (0.804 g, 2.59 mmol) and tert-butyl 4-(ethylamino)piperidine-1-carboxylate (0.6477 g, 2.84 mmol) in DIPEA (0.680 ml, 3.89 mmol) and DMF (5.00 ml, 2.59 mmol) was added (1-(chloro-1-pyrrolidinylmethylene)pyrrolidinium hexafluorophosphate) (0.9484 g, 2.85 mmol), and stirred for overnight before another portion of tert-butyl 4-(ethylamino)piperidine-1-carboxylate (0.2654 g, 1.16 mmol) was added. The reaction mixture was stirred for overnight and partitioned into AcOEt (200 ml) and sat'd NaHCO₃ aq. (100 ml), and the aqueous layer was extracted with AcOEt. The combined organic layer was washed with sat'd NaHCO₃ aq. (100 ml) and sat'd NaCl aq. (100 ml), and dried over Na₂SO₄. The solvent was removed under reduced pressure and chromatographed on silica (CH₂C₂→CH₂Cl₂/MeOH=10/1) then (CH₂Cl₂+>AcOEt) to yield title product as an off-white solid.

### Example 7

### Synthesis of (R)-N-ethyl-2-(3-oxo-1-tosyl-1,2,3,4-tetrahydropyrazin-2-yl)-N-(piperidin-4-yl)acetamide

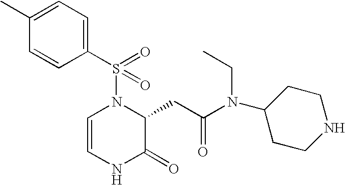

A solution of (R)-tert-butyl 4-(N-ethyl-2-(3-oxo-1-tosyl-1,2,3,4-tetrahydropyrazin-2-yl)acetamido)piperidine-1-carboxylate (0.0203 g, 0.0390 mmol) in hydrogen chloride solution (4M in 1,4-dioxane, 1.00 ml, 4.00 mmol) and 1,4-dioxane (0.500 ml) was stirred overnight. The reaction mixture was condensed under reduced pressure and the remaining solid was suspended with Et₂O and the resulting solid was washed with Et₂O and dried in vacuo to yield the title product as HCl salt.

### Example 8

### Synthesis of N-((S)-1-cyclopropyl-3,3-dimethylpiperidin-4-yl)-2-((R)-3-oxo-1-tosyl-1,2,3,4-tetrahydropyrazin-2-yl)acetamide N-oxide

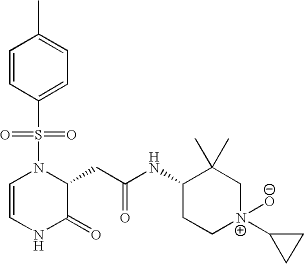

To a solution of N-((S)-1-cyclopropyl-3,3-dimethylpiperidin-4-yl)-2-((R)-3-oxo-1-tosyl-1,2,3,4-tetrahydropyrazin-2-yl)acetamide (110 mg, 239 μmol) in MeCN (10 ml) was added 3-chloroperoxybenzoic acid (49 mg, 287 μmol). After stirring at RT for 1 h, the solvent was evaporated to dryness and the residue was directly submitted to column chromatography (SiO₂, EtOAc/MeOH=5:1 to EtOAc/2M NH₃ in MeOH=5:1 to 3:1 to 2:1) to give the title compound as a white solid. MS: 477 (M+1).

### Example 9

### Synthesis of (R)-4-(4-chloro-2,5-dimethylphenylsulfonyl)-3-(2-(4-(hydroxymethyl)piperidin-1-yl)-2-oxoethyl)-3,4-dihydropyrazin-2(1H)-one

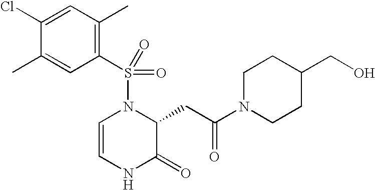

A solution of (R)-2-(1-(4-chloro-2,5-dimethylphenylsulfonyl)-3-oxo-1,2,3,4-tetrahydropyrazin-2-yl)acetic acid (100 mg, 279 Amos), piperidin-4-ylmethanol (64 mg, 557 mmol), 1-hydroxybenzotriazole (38 mg, 279 μmol) and n-(3-dimethylaminopropyl)-n′-ethylcarbodiimide hydrochloride (53 mg, 279 Amok) in 1 mL of DMF for 12 h. The reaction mixture was quenched with sat. NaHCO₃, extracted with EtOAc/hexane=2:1, washed with brine, dried over Na₂SO₄, filtered and evaporated to dryness. Flash chromatography (SiO₂, EtOAc to EtOAc/MeOH 100:5 to 100:10) afforded the title compound as a sticky oil. MS: 456 (M⁺).

### Example 10

### Synthesis of (R)-4-(4-chloro-2,5-dimethylphenylsulfonyl)-3-(2-oxo-2-(4-(pyrrolidin-1-ylmethyl)piperidin-1-yl)ethyl)-3,4-dihydropyrazin-2(1H)-one

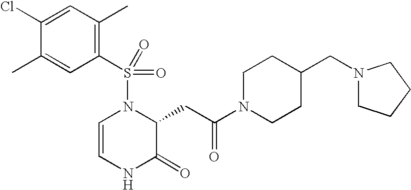

### Step 1

To a solution of (R)-4-(4-chloro-2,5-dimethylphenylsulfonyl)-3-(2-(4-(hydroxymethyl)-piperidin-1-yl)-2-oxoethyl)-3,4-dihydropyrazin-2(1H)-one (46 mg, 101 μmol) in DCM (10 ml) was added triethylamine (31 mg, 303 μmol) and methanesulfonyl chloride (23 mg, 202 μmol). After stirring at RT for 10 min, the reaction mixture was directly loaded onto column (SiO2, EtOAc to EtOAc/MeOH=100:7) to give (R)-(1-(2-(1-(4-chloro-2,5-dimethylphenyl-sulfonyl)-3-oxo-1,2,3,4-tetrahydropyrazin-2-yl)acetyl)piperidin-4-yl)-methyl methanesulfonate as a film. MS: 534 (M⁺).

### Step 2

A solution of (R)-(1-(2-(1-(4-chloro-2,5-dimethylphenylsulfonyl)-3-oxo-1,2,3,4-tetrahydropyrazin-2-yl)acetyl)piperidin-4-yl)methyl methanesulfonate (40 mg, 75 μmol) and pyrrolidine (53 mg, 749 μmol) in DCM was stirred at RT for 36 h. The solvent was evaporated and the residue was loaded on prep TLC (SiO₂, EtOAc/MeOH=100:30) to give the title compound as a film. MS: 509 (M⁺).

### Formulations

The following are representative pharmaceutical formulations containing a compound of formula (I).

### Tablet Formulation

The following ingredients are mixed intimately and pressed into single scored tablets.

| | Quantity per |
|---|---|
| Ingredient | tablet, mg |
| compound of this invention | 400 |
| corn starch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution | 12.85 | g |
| Veegum K (Vanderbilt Co. | 1.0 | g |
| flavoring | 0.035 | ml |
| colorings | 0.5 | mg |
| distilled water | q.s. to 100 | ml |

### Injectable Formulation

The following ingredients are mixed to form an injectable formulation.

| Ingredient | Amount | |
|---|---|---|
| compound of this invention | 0.4 | mg |
| sodium acetate buffer solution, 0.4 M | 2.0 | ml |
| HCl (1N) or NaOH (1N) | q.s. to suitable pH | |
| water (distilled, sterile) | q.s. to 20 | ml |

### Biological Testing

### Example 1

### Radioligand Binding Assay for Human B1 and Human B2 Bradykinin Receptor

Step 1 Preparation of membranes expressing human B1 bradykinin receptor:

Membranes were prepared from CHO-d-AQN cells stably transfected with human bradykinin B1 receptor cDNA. For large-scale production of membranes, cells were grown in 100 L suspension culture to 1.0E8 cells/mL then harvested using the Viafuge at continuous centrifugation of 1000 g. For pilot studies, cells were grown in 2 L spinner culture and harvested by centrifugation (1900 g, 10 min, 4° C.). The cell pellet was washed with PBS, centrifuged (1900 g, 10 min, 4° C.), then the cells resuspended in lysis buffer (25 mM HEPES, pH 7.4, 5 mM EDTA, 5 mM EGTA, 3 mM MgCl₂, 10% (w/v) sucrose, Complete Protease Inhibitor tablets (EDTA-free)) to a density of 14% w/v for passage through a microfluidizer (Microfluidics 110S, 3 passes, 6,000 psi). The resulting cell lysate was centrifuged (1900 g, 10 min, 4° C.), and the crude particulate fraction isolated by centrifugation (142,000 g, 1 h, 4° C.) of the low-speed supernatant. The resulting pellet was resuspended in ⅓ the original lysis buffer volume, homogenized, and recentrifuged as above. The membrane pellet was resuspended by homogenization in storage buffer (25 mM HEPES, pH 7.4, 3 mM MgCl₂, 10% (w/v) sucrose and Complete Protease Inhibitor tablets (EDTA-free)). Single-use aliquots were made and flash-frozen in liquid N₂ prior to storage at −80° C.

Membranes containing human bradykinin B2 receptor were purchased from Receptor Biology (now Perkin Elmer Life Sciences). They were derived from a CHO-K1 line stably expressing the human B2 receptor developed by Receptor Biology and subsequently purchased by Amgen. For some studies, membranes were prepared in-house from this same cell line using the method described for human B1 receptor membranes, except cells were grown in roller bottles and harvested using Cellmate.

Step 2 Human B1 receptor binding assay was performed in 96-well polypropylene plates (Costar 3365) by adding 50 μl [³H]des-arg¹⁰ kallidin (NET1064; Perkin Elmer Life Sciences) to 10 μL test compound diluted in 90 μL assay buffer (24 mM TES, pH 6.8, 1 mM 1,10 o-phenanthroline, 0.3% BSA, 0.5 mM Pefabloc SC, 2 μg/mL aprotinin, 5 μg/mL leupeptin, and 0.7 μg/mL pepstatin A). Membranes (50 μL) were added last. [³H] des-arg¹⁰ kallidin was diluted from stock into assay buffer to yield a final concentration of ˜0.3 nM in the assay but was adjusted as, needed to ensure a concentration at or below the K_{d} determined for each batch of receptor membranes. Nonspecific binding was defined with 2 μM des-Arg¹⁰Leu⁹ kallidin. Membranes were diluted in assay buffer to yield a final concentration of 0.068 nM hB1 receptor in the assay. Compounds were solubilized in either DMSO or ddH₂0, plated into polypropylene plates (Costar 3365), then serially diluted in either DMSO or dilution buffer (20 mM Hepes, pH 7.6, 0.1% BSA) to yield a final concentration of either 5% DMSO or no DMSO in the assay. The assay reaction mixture was incubated with shaking for 1 h at RT and then filtered through GF/C plates presoaked in 0.5% polyethyleneimine (Unifilter; Perkin Elmer Life Sciences) using a Filtermate 96-well harvester (Perkin Elmer Life Sciences). Filter plates were rapidly washed 6 times with 200 μL ice-cold buffer (50 mM Tris, pH 7.4), dried in a vacuum oven at 55° C. for 15-20 min, backed, and 40 μL per well of Microscint 20 was added. The plates were sealed and activity read on Topcount (Perkin Elmer Life Sciences) using a count time of 3 min per channel.

For human B2 bradykinin receptor, the same procedure was followed with the following exceptions: [³H] bradykinin (NET706; Perkin Elmer Life Sciences) was used at a final concentration of ˜0.2 nM and non-specific binding was defined with 2 μM bradykinin. Human B2 receptor concentration was 0.068 nM final in the assay.

### Data Analysis

Data was analyzed in XLFit with the four-parameter logistic y=A+((B−A)/(1+((C/x)̂D))) and fit with the Levenburg-Marquardt algorithm. Raw cpm were converted to percent of control values prior to analysis (POC=((compound cpm−nonspecific cpm)/(no-compound cpm-nonspecific cpm)*100)). K; values were determined from the IC₅₀ using the Cheng-Prusoff and K_{d} values determined by direct saturation binding of the radioligands.

### Example 2

### In vitro B—Inhibition Activity

In vitro Assay of human B1 Receptor Function using Calcium Flux

Activation of the G_{q} linked B1 receptor results in an increase in intracellular calcium. The calcium sensitive photoprotein aequorin can, therefore, be used as an indicator of B1 receptor activation. Aequorin is a 21-kDa photoprotein that forms a bioluminescent complex when linked to the chromophore cofactor coelenterazine. Following the binding of calcium to this complex, an oxidation reaction of coelenterazine results in the production of apoaequorin, coelenteramide, CO₂, and light that can be detected by conventional luminometry.

A stable CHO D-/hB1/Aequorin cell line was established and the cells were maintained in suspension in spinner bottles containing a 1:1 ratio of DMEM and HAM F12 (Gibco 11765-047), high glucose (Gibco 11965-084), 10% Heat Inactivated Dialyzed serum (Gibco 26300-061), 1× Non-Essential Amino Acids (Gibco 11140-050), 1× Glutamine-Pen-Strep (Gibco 10378-016), and Hygromycin, 300 μg/mL (Roche 843555). 15-24 h prior to the luminometer assay, 25,000 cells/well (2.5E6 cells/10 mL/plate) were plated in 96-well black-sided clear bottom assay plates (Costar #3904).

Media was removed from the wells and replaced with 60 μL of serum free HAM's F12 with 30 mM HFPES (pH 7.5) and 15CM coelenterazine (Coelenterazine h Luciferin #90608 from Assay Designs). The plates were incubated for 1.5-2 h. Ten point IC₅₀ compound plates containing 1:3 or 1:5 dilutions of antagonist compounds and an agonist activator plate (20 nM des-Arg10-Kallidin final concentration, EC₈₀) were prepared using Ham's F12 with 30 mM HEPES, pH 7.5. Following coelenterazine incubation, an automated flash-luminometer platform was used to dispense the B1 antagonist compounds (dissolved in DMSO and diluted with buffer to the desired concentration (final DMSO concentration <1% DMSO)) to the cell plate, a CCD camera situated underneath the cell plate took 12 images of the cell plate at 5 second intervals to determine if there was any agonist activity with the compounds. The hB1 agonist, des-Arg₁₀-Kallidin, was added to the cell plate and another 12 images were recorded to determine the IC₅₀ of the antagonist(s).

In vitro Assay of hB2 Receptor Function using Calcium Flux

The intracellular calcium flux induced by hB2 receptor activation was analyzed using an hB2 recombinant cell line (CHO-K1) purchased from PerkinElmer (Catalog Number: RBHB2C000EA) on a fluorometric imaging plate reader (FLIPR). The cells were cultured in T225 flask containing Ham's F12 Nutrient Mixture (Invitrogen Corp., Cat # 11765-047), 10% Fetal Clone II Bovine Serum (HyClone, Cat # SH3006603), 1 mM Sodium pyruvate (100 mM stock, Invitrogen Corp., Cat# 12454-013), and 0.4 mg/mL Geneticin (G418; 50 mg/mL active geneticin, Invitrogen, Cat# 10131-207). Culture medium was changed every other day. 24 h prior to the FLIPR assay, the hB2/CHO cells were washed once with PBS (Invitrogen) and 10 mL of Versene (1:5000, Invitrogen, Cat# 15040-066) was added to each flask. After 5 min incubation at 37° C., Versene was removed and cells were detached from the flask and resuspended in culture medium. Cells were counted and 25,000 cells/well were plated in 96-well black-sided clear bottom assay plates (Costar #3904). Cells were incubated in a 37° C. CO₂ incubator overnight.

The media was aspirated from the cells and replaced with 65 μL of dye-loading buffer. The loading buffer was prepared by diluting a stock solution of 0.5 mM Fluo-4 AM (Molecular Probes, dissolved in DMSO containing 10% [w/v] pluronic acid) to a concentration of 1 μM in Clear Dulbecco's Modified Eagle Medium (DMEM) containing 0.1% BSA, 20 mM HEPES, and 2.5 mM probenecid. The cells were dye-loaded for 1 h at RT. The excess dye was removed by washing the cells 2× with assay buffer. The assay buffer consists of Hank's Balanced Salt Solution (HBSS) containing 20 mM HEPES, 0.1% BSA, and 2.5 mM probenecid. After the wash cycles, a volume of 100 μL was left in each well, and the plate was ready to be assayed in the FLIPR System. Single point (10 μM final concentration) POC antagonist compound plates or ten point IC₅₀ compound plates containing 1:3 or 1:5 dilutions of antagonist compounds (dissolved in DMSO and diluted with buffer to the desired concentration (final DMSO concentration <1% DMSO)) and an agonist activator plate (0.3 nM bradykinin final concentration, EC₈₀) were prepared using assay buffer. The cell plate and the compound plates were loaded onto the FLIPR and during the assay, fluorescence readings are taken simultaneously from all 96 wells of the cell plate. Ten 1-second readings were taken to establish a stable baseline for each well, then 25 μL from the B1 antagonist plate was rapidly (50 μL/sec.) added. The fluorescence signal was measured in 1-second (1 min) followed by 6-second (2 min) intervals for a total of 3 min to determine if there is any agonist activity with the compounds. The B2 agonist, bradykinin, was added to the cell plate and another 3 min were recorded to determine the percent inhibition at 10 μM (POC plates) or the IC₅₀ of the antagonist. The activity of some of the compounds of this invention in this assay is given in the table below.

| Cpd. # | Data (um) | Cpd. # | Data (um) |
|---|---|---|---|
| T-1-7 | 0.70 | T-1-8 | 0.17 |
| T-1-65 | 2.7 | T-1-11 | 0.53 |
| T-1-71 | 2.0 | T-1-15 | 0.43 |
| T-1-17 | 0.001 | T-1-18 | 0.015 |
| T-1-21 | 0.0011 | T-1-25 | 0.00121 |
| T-1-42 | 1.98 | T-3-43 | 2.6 |
| T-1-44 | 2.3 | T-3-1 | 0.85 |
| T-1-46 | >4 | T-3-47 | 0.39 |
| T-3-2 | 0.13 | T-3-3 | 1.0 |
| T-3-48 | 1.6 | T-3-50 | >4 |
| T-1-48 | 0.4 | T-1-59 | 0.025 |
| T-1-30 | 0.19 | T-1-31 | 0.047 |
| T-1-34 | 0.14 | | |
| T-2-1 | 0.003 | T-1-35 | 0.42 |
| T-1-100 | >4 | T-1-37 | 0.019 |
| T-1-40 | 0.2 | T-1-111 | 0.0014 |
| T-1-41 | 0.002 | T-1-43 | 0.014 |
| T-1-45 | 0.001 | T-1-46 | 0.0037 |
| T-1-47 | 0.0008 | | |
| T-1-50 | 0.0013 | T-1-57 | 0.001 |
| T-1-67 | 2.2 | T-1-68 | 2.2 |
| T-3-5 | 0.13 | T-1-120 | 0.001 |
| T-1-15 | 0.15 | T-1-77 | 0.02 |
| T-1-114 | na | T-3-8 | 0.27 |
| T-3-10 | 0.27 | T-3-15 | 0.94 |
| T-3-18 | 0.16 | T-3-22 | 0.038 |
| T-3-24 | 0.036 | T-3-26 | 0.015 |
| T-3-28 | 0.032 | T-3-30 | 0.061 |
| T-3-32 | 0.0015 | T-3-38 | 0.0051 |
| T-1-2 | 0.092 | T-1-58 | 0.020 |
| T-1-3 | 0.021 | T-1-4 | 0.021 |
| T-3-40 | >4 | T-1-107 | 0.42 |

For the purposes of this table, T-1-7 means Table 1, compound 7.

### Example 3

### Cell and Tissue based In Vitro Assays of hB1 Receptor Binding

These studies established the antagonist activity of several compounds at the bradykinin B1 receptors in in vitro cell-based and isolated organ assays.

1. Rabbit endothelial cell B1-specific PGI₂ secretion Assay
2. B1 and B2 umbilical vein Assay
In vitro B1—Inhibition Activity:

The effectiveness of the compounds as inhibitors of B1 activity (i.e., B1 “neutralization”) can be evaluated by measuring the ability of each compound to block B1 stimulated CGRP and substance P release and calcium signaling in Dorsal Root Ganglion (DRG) neuronal cultures.

### Dorsal Root Ganglion Neuronal Cultures:

Dorsal root ganglia are dissected one by one under aseptic conditions from all spinal segments of embryonic 19-day old (E19) rats that are surgically removed from the uterus of timed-pregnant, terminally anesthetized Sprague-Dawley rats (Charles River, Wilmington, Mass.). DRG are collected in ice-cold L-15 media (GibcoBRL, Grand Island, N.Y.) containing 5% heat inactivated horse serum (GibcoBRL), and any loose connective tissue and blood vessels are removed. The DRG are rinsed twice in Ca²⁺- and Mg²⁺-free Dulbecco's phosphate buffered saline (DPBS), pH 7.4 (GibcoBRL). The DRG are dissociated into single cell suspension using a papain dissociation system (Worthington Biochemical Corp., Freehold, N.J.). Briefly, DRG are incubated in a digestion solution containing 20 U/mL of papain in Earle's Balanced Salt Solution (EBSS) at 37° C. for fifty minutes. Cells are dissociated by trituration through fire-polished Pasteur pipettes in a dissociation medium consisting of MEM/Ham's F12, 1:1, 1 mg/mL ovomucoid inhibitor and 1 mg/mL ovalbumin, and 0.005% deoxyribonuclease I (DNase). The dissociated cells are pelleted at 200×g for 5 min and re-suspended in EBSS containing 1 mg/mL ovomucoid inhibitor, 1 mg/mL ovalbumin and 0.005% DNase. Cell suspension is centrifuged through a gradient solution containing 10 mg/mL ovomucoid inhibitor, 10 mg/mL ovalbumin at 200×g for 6 min to remove cell debris, then filtered through a 88-μM nylon mesh (Fisher Scientific, Pittsburgh, Pa.) to remove any clumps. Cell number is determined with a hemocytometer, and cells are seeded into poly-ornithine 100 μg/mL (Sigma, St. Louis, Mo.) and mouse laminin 1 mg/mL (GibcoBRL)-coated 96-well plates at 10×10³ cells/well in complete medium. The complete medium consists of minimal essential medium (MEM) and Ham's F12, 1:1, penicillin (100 U/mL), streptomycin (100 μg/mL), and 10% heat inactivated horse serum (GibcoBRL). The cultures are kept at 37° C., 5% CO₂ and 100% humidity. For controlling the growth of non-neuronal cells, 5-fluoro-2′-deoxyuridine (75CM) and uridine (180 μM) are included in the medium.

Two hours after plating, cells are treated with recombinant human β-bl or recombinant rat β-b1 at a concentration of 10 mg/ml (0.38 nm a). Positive controls comprising serial-diluted anti-b1 antibody (r&d systems, Minneapolis, mn) are applied to each culture plate. Compounds are added at ten concentrations using 3.16-fold serial dilutions. All samples are diluted in complete medium before being added to the cultures. Incubation time is generally around 40 h prior to measurement of vrl expression.

### Measurement of VR1 Expression in DRG Neurons:

Cultures are fixed with 4% paraformaldehyde in Hanks' balanced salt solution for 15 min, blocked with Superblock (Pierce, Rockford, Ill.), and permeabilized with 0.25% Nonidet P-40 (Sigma) in Tris.HCl (Sigma)-buffered saline (TBS) for 1 h at RT. Cultures are rinsed once with TBS containing 0.1% Tween 20 (Sigma) and incubated with rabbit anti-VR1 IgG (prepared at Amgen) for 1.5 h at RT, followed by incubation of Eu-labeled anti-rabbit second antibody (Wallac Oy, Turku, Finland) for 1 h at RT. Washes with TBS (3× five min with slow shaking) are applied after each antibody incubation. Enhance solution (150 mL/well, Wallac Oy) is added to the cultures. The fluorescence signal is measured in a time-resolved fluorometer (Wallac Oy). VR1 expression in samples treated with the compounds is determined by comparing to a standard curve of B1 titration from 0-1000 ng/mL. Percent inhibition (compared to maximum possible inhibition) of B1 effect on VR1 expression in DRG neurons is determined by comparing to controls that are not B1-treated.

### Example 4

### In Vivo Antinociceptive Activity in Rat Model

### Rat Neuropathic Pain Model

Male Sprague-Dawley rats (200 g) are anesthetized with isoflurane inhalant anesthesia and the left lumbar spinal nerves at the level of L5 and L6 are tightly ligated (4-0 silk suture) distal to the dorsal root ganglion and prior to entrance into the sciatic nerve, as first described by Kim and Chung (Kim, S. H.; Chung, J. M. An experimental model for peripheral neuropathy produced by segmental spinal nerve ligation in the rat. Pain 50:355-363, (1992)). The incisions are closed and the rats are allowed to recover. This procedure results in mechanical (tactile) allodynia in the left hind paw as assessed by recording the pressure at which the affected paw (ipsilateral to the site of nerve injury) was withdrawn from graded stimuli (von Frey filaments ranging from 4.0 to 148.1 mN) applied perpendicularly to the plantar surface of the paw (between the footpads) through wire-mesh observation cages. A paw withdrawal threshold (PWT) was determined by sequentially increasing and decreasing the stimulus strength and analyzing withdrawal data using a Dixon non-parametric test, as described by Chaplan et al. (Chaplan, S.R.; Bach, F. W.; Pogrel, J. W.; Chung, J. M.; Yaksh, T. L. Quantitative assessment of tactile allodynia in the rat paw. J. Neurosci. Meth., 53:55-63 (1994)).

Normal rats and sham surgery rats (nerves isolated but not ligated) withstand at least 148.1 mN (equivalent to 15 g) of pressure without responding. Spinal nerve ligated rats respond to as little as 4.0 mN (equivalent to 0.41 g) of pressure on the affected paw. Rats are included in the study only if they did not exhibit motor dysfunction (e.g., paw dragging or dropping) and their PWT was below 39.2 in N (equivalent to 4.0 g). At least seven days after surgery rats are treated with compounds (usually a screening dose of 60 mg/kg) or control diluent (PBS) once by s.c. injection and PWT was determined each day thereafter for 7 days.

### Rat CFA Inflammatory Pain Model

Male Sprague-Dawley rats (200 g) are lightly anesthetized with isoflurane inhalant anesthesia and the left hind paw is injected with complete Freund's adjuvant (CFA), 0.15 mL. This procedure results in mechanical (tactile) allodynia in the left hind paw as assessed by recording the pressure at which the affected paw is withdrawn from graded stimuli (von Frey filaments ranging from 4.0 to 148.1 mN) applied perpendicularly to the plantar surface of the paw (between the footpads) through wire-mesh observation cages. PWT is determined by sequentially increasing and decreasing the stimulus strength and analyzing withdrawal data using a Dixon non-parametric test, as described by Chaplan et al. (1994). Rats are included in the study only if they do not exhibit motor dysfunction (e.g., paw dragging or dropping) or broken skin and their PWT is below 39.2 mN (equivalent to 4.0 g). At least seven days after CFA injection rats are treated with compounds (usually a screening dose of 60 mg/kg) or control solution (PBS) once by s.c. injection and PWT is determined each day thereafter for 7 days. Average paw withdrawal threshold (PWT) is converted to percent of maximum possible effect (% MPE) using the following formula: % MPE=100*(PWT of treated rats−PWT of control rats)/(15-PWT of control rats). Thus, the cutoff value of 15 g (148.1 mN) is equivalent to 100% of the MPE and the control response is equivalent to 0% MPE.

At the screening dose of 60 mg/kg, compounds in vehicle are expected to produce an antinociceptive effect with a PD relationship.

The foregoing is merely illustrative of the invention and is not intended to limit the invention to the disclosed compounds. All mentioned references, patents, applications and publications, are hereby incorporated by reference in their entirety, as if here written.

1. A compound of Formula (I): 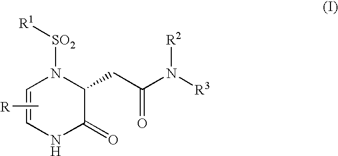
wherein:
R is hydrogen or alkyl;
R¹ is aryl or heteroaryl optionally substituted with R^{a}, R^{b}, and R^{c} independently selected from alkyl, halo, haloalkyl, haloalkoxy, hydroxy, cyano, alkylamino, dialkylamino, or alkoxy;
R² is hydrogen, alkyl, haloalkyl, cycloalkyl, cycloalkylalkyl, hydroxyalkyl, alkoxyalkyl, or aminoalkyl;
R³ is bridged heterocyclyl, bridged cycloalkyl wherein bridged heterocyclyl or bridged cycloalkyl is optionally substituted with one, two or three substitutents independently selected from alkyl, alkoxy, hydroxyl, hydroxyalkyl, or alkoxyalkyl; or R³ is a group of formula (a): 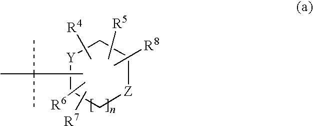
where:
n is 0, 1,2, or 3;
Y is —CH₂— or —NH—;
Z is —C(O)—, —C(═NOH), —CONH—, —O—, —C(O)O—, —S—, —SO—, —SO₂—, —NH—, or —CH₂—;
R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano; and
R⁷ and R⁸ are independently hydrogen or alkyl; or
R⁴ and R⁷, when attached to the same carbon atom, can combine together with the carbon atom to which they are attached to form a saturated or unsaturated monocyclic (C₃-C₈)cycloalkyl optionally substituted with R^{d}, R^{e}, and R^{f} as defined above; or a saturated or unsaturated monocyclic heterocyclyl ring containing three to six ring atom wherein one or two ring atoms are selected from —C(O)—, —C(═NOH), —O—, —S—, —SO—, —SO₂—, or —NH— and wherein the heterocyclyl ring is substituted with R^{m}, Rⁿ and R^{o} where R^{m} and Rⁿ are independently selected from hydrogen, alkyl, hydroxyl, alkoxy, halo, haloalkyl, haloalkoxy, carboxy, or alkoxycarbonyl and R^{o} is selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy and where the aryl, heteroaryl, or heterocyclyl ring in R^{o} is optionally substituted with R^{d}, R^{e}, and R^{f} as defined above; provided that when Y and Z are —CH₂— and R⁴ and R⁷ do not form a cycloalkyl or heterocyclyl ring, then at least one of R⁴, R⁵, R⁶, R⁷, and R⁸ is not hydrogen; or
R² and R³ together with the nitrogen atom to which they are attached form monocyclic heterocyclyl or spiro heterocycloamino each of which is substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R^{g}, R^{h}, and Rⁱ is optionally substituted with R^{j}, R^{k}, or R^{l} independently selected from alkyl, halo, alkoxy, haloalkyl, hydroxyl, carboxy, alkoxycarbonyl, haloalkoxy, or cyano provided that when R² and R³ together with the nitrogen atom to which they are attached form monocyclic heterocyclyl, then at least one of R^{g}, R^{h}, and Rⁱ is not hydrogen;
or an N-oxide; and/or a pharmaceutically acceptable salt thereof provided that: (i) when R is hydrogen, R¹ is 3,4-dichlorophenyl then R² and R³ together with the nitrogen atom to which they are attached do not form 2- and 3-phenylpyrrolidin-1-yl, 2- and 3-benzylpyrrolidin-1-yl, 2-phenylpiperidin-1-yl, 4-phenylpiperidin-1-yl, 4-phenylpiperazin-1-yl, 2-, 3- and 4-hydroxymethylpiperidin-1-yl, 2-, 3-, and 4-benzylpiperidin-1-yl, 2- 3- and 4-methylpiperidin-1-yl, 3-phenylpiperidin-1-yl, azabicyclo[3.2.2]non-3-yl, azabicyclo[3.2.1]oct-6-yl, 1,3,3-trimethylazabicyclo[3.2.1]oct-6-yl, 2-phenylazepin-1-yl, 4-(pyridin-2-yl)piperazin-1-yl, 3-, or 4-(cyanomethyl)piperazin-1-yl, 4-hydroxyazepan-1-yl, or 4-hydroxy-4-methylazepan-1-yl (ii) when R is hydrogen, R¹ is 4-methylphenyl then R² and R³ together with the nitrogen atom to which they are attached do not form 4-(2-hydroxyethyl)piperazin-1-yl, 2-(4-methoxycarbonylphenyl)pyrrolidin-1-yl, 2-(4-methoxycarbonylphenyl)piperidin-1-yl, 2-(4-hydroxymethylphenyl)piperidin-1-yl, 4-(4-methylpiperazin-1-yl)piperidin-1-yl, 4-(4-phenylpiperidin-1-yl)piperidin-1-yl, 2-(4-piperidin-1-ylmethylphenyl)piperidin-1-yl, or 5-oxa-2-aza-bicyclo[2.2.1]heptan-2-yl (iii) when R is hydrogen, R¹ is 2,3-dichlorophenyl, then R² and R³ together with the nitrogen atom to which they are attached do not form 4-benzylpiperazin-1-yl, 2-phenylpyrrolidin-1-yl, 2-(4-piperidin-1-ylmethylphenyl)piperidin-1-yl, 2-(3-piperidin-1-ylmethylphenyl)piperidin-1-yl, 2-(4-piperidin-1-ylmethylphenyl)-pyrrolidin-1-yl, 2-(3-piperidin-1-ylmethylphenyl)pyrrolidin-1-yl, or 2-phenyl-piperidin-1-yl (iv) when R is hydrogen, R¹ is 4-methylphenyl, and R² is hydrogen then R³ is not 1-benzylpiperidin-4-yl, 2- 3-, and 4-methylcyclohexyl, 2- 3-, and 4-hydroxycyclohexyl, 2-benzyloxycyclohexyl, 2-ethyloxycarbonylcyclohexyl, tricyclo[3.3.1.1˜3,7]dec-1-yl, 3-hydroxytricyclo[3.3.1.1˜3,7]dec-1-yl, admant-1-yl, or 2- or 3-hydroxymethylcyclohexyl, and (v) when R is hydrogen, R¹ is 2,5-dimethyl-phenyl, and R² is hydrogen then R³ is not 2-methylcyclohexyl.

2. The compound of claim 1 wherein:
R¹ is aryl or heteroaryl optionally substituted with R^{a}, R^{b}, and R^{c} independently selected from alkyl, or halo;
R² is hydrogen, alkyl, cycloalkyl, cycloalkylalkyl, hydroxyalkyl, alkoxyalkyl, or aminoalkyl;
R³ is bridged heterocyclyl, bridged cycloalkyl wherein bridged heterocyclyl, or bridged cycloalkyl ring is optionally substituted with one, two or three substitutents independently selected from alkyl, alkoxy, hydroxyl, hydroxyalkyl, or alkoxyalkyl; or a group of formula (a): 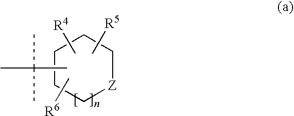
where:
n is 0, 1, 2, or 3;
Z is —C(O)—, —C(═NOH), —CONH—, —O—, —C(O)O—, —S—, —SO—, —SO₂—, —NH—, or —CH₂—; and
R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano provided that when Z is —CH₂— then at least one of R⁴, R⁵, and R⁶ is not hydrogen; or
R² and R³ together with the nitrogen atom to which they are attached form monocyclic heterocyclyl optionally substituted with R^{g}, R^{h}, and Rⁱ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R^{g}, R^{h}, and Rⁱ is optionally substituted with R^{j}, R^{k}, and R^{l} independently selected from alkyl, halo, alkoxy, haloalkyl, hydroxyl, carboxy, alkoxycarbonyl, haloalkoxy, or cyano provided that at least one of R^{g}, R^{h}, and Rⁱ is not hydrogen.

3. The compound of claim 1 wherein R² is hydrogen.

4. (canceled)

5. The compound of claim 2 wherein R² is hydrogen.

6. The compound of claim 3 wherein R³ is bridged heterocyclyl optionally substituted with one, two or three substitutents independently selected from alkyl, alkoxy, hydroxyl, hydroxyalkyl, or alkoxyalkyl.

7. The compound of claim 5 wherein R³ is cyclopentyl or cyclohexyl substituted with R⁴, R⁵, and R⁶ independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano and R⁷ and R⁸ are hydrogen.

8. The compound of claim 3 wherein R³ is a group of formula: 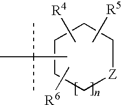
where n is 0, 1, 2 or 3 and Z is —NH— and R⁴, R⁵, and R⁶ are independently selected from hydrogen, alkyl, hydroxyl, alkoxy, halo, haloalkyl, haloalkoxy, acyl, alkoxycarbonyl, alkoxyalkyl, alkoxyalkyloxy, hydroxyalkyl, hydroxyalkyloxy, aminoalkyl, aminoalkoxy, carboxyalkyl, alkoxycarbonylalkyl, cyanoalkyl, cycloalkyl, cycloalkylalkyl, amino, monosubstituted amino, disubstituted amino, aryl, aralkyl, aryloxy, aralkyloxy, heteroaryl, heteroaryloxy, heteroaralkyl, heteroaralkyloxy, heterocyclyl, heterocyclyloxy, heterocyclylalkyl or heterocyclylalkyloxy where the aryl, heteroaryl, or heterocyclyl ring in R⁴, R⁵, and R⁶ is optionally substituted with R^{d}, R^{e}, and R^{f} independently selected from alkyl, halo, alkoxy, haloalkyl, hydroxyl, carboxy, alkoxycarbonyl, haloalkoxy, or cyano and R⁷ and R⁸ are hydrogen.

9. The compound of claim 3 wherein R³ is a group of formula: 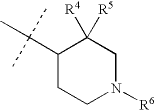
wherein R⁴ and R⁵ are independently hydrogen or alkyl provided that at least one is alkyl.

10. The compound of claim 9 wherein R⁶ is hydrogen, alkyl, haloalkyl, alkoxycarbonyl, cycloalkyl, cycloalkylalkyl, aralkyl, heteroaralkyl, heteroaryl, aryl, acyl, aminoalkyl, or alkoxyalkyl.

11. (canceled)

12. (canceled)

13. (canceled)

14. (canceled)

15. (canceled)

16. The compound of claim 3 wherein R³ is a group of formula (a) where R⁴ and R⁷ combine together with the carbon atom to which they are attached to form a saturated or unsaturated monocyclic heterocyclyl ring containing three to six ring atom wherein one or two ring atoms are selected from —C(O)—, —O—, —S—, —SO—, —SO₂—, or —NH— and wherein the heterocyclic ring is substituted with R^{m}, Rⁿ and R^{o} as defined above.

17. (canceled)

18. The compound of claim 3 wherein R² and R³ together with the nitrogen atom to which they are attached form spiro heterocycloamino each of which is substituted with R^{g}, R^{h}, and Rⁱ as defined above.

19. The compound of claim 8 wherein R is hydrogen and R¹ is phenyl substituted with R^{a}, R^{b}, or R^{c} independently selected from hydrogen, alkyl, or halo.

20. (canceled)

21. (canceled)

22. The compound of claim 2 wherein R and R² are hydrogen and R¹ is 4-methylphenyl, 2,3-dichlorophenyl, 3,4-dichlorophenyl, and 2,5-dimethyl-4-chlorophenyl, and R³ is 3,3-dimethylpiperidin-4-yl, 1-methyl-3,3-dimethylpiperidin-4-yl, 1-(isobutyl)-3,3-dimethylpiperidin-4-yl, 1-(pyridin-2-yl-methyl)-3,3-dimethylpiperidin-4-yl, 1-(2-propyl)-3,3-dimethylpiperidin-4-yl, 1-(pyridin-4-ylmethyl)-3,3-dimethylpiperidin-4-yl, 1-(acetyl)-3,3-dimethylpiperidin-4-yl, 1-(tert-butyoxycarbonyl)-3,3-dimethyl-piperidin-4-yl, 1-cyclopropyl-3,3-dimethylpiperidin-4-yl, 1-(pyridin-3-ylmethyl)-3,3-dimethylpiperidin-4-yl, 1-(2-methoxyethyl)-3,3-dimethylpiperidin-4-yl, 3,3-dimethyl-1-(2,2,2-trifluoroethyl)-piperidin-4-yl, 1-(tert-butyoxy-carbonyl)piperidin-3-yl, 1-ethyl-3,3-dimethyl-piperidin-4-yl, 1-n-propyl-3,3-dimethylpiperidin-4-yl, 1-2,2-dimethylpropyl-3,3-dimethylpiperidin-4-yl, 1-cyclobutyl-3,3-dimethylpiperidin-4-yl, or 1-cyclopentyl-3,3-dimethylpiperidin-4-yl wherein the stereochemistry at the carbon atom at the 4-position of the piperidin-4-yl ring is (R), (S), or (RS).

23. A compound selected from:
(3R)-3-(2-(3-azabicyclo[3.2.2]non-3-yl)-2-oxoethyl)-4-((3,4-dichlorophenyl)-sulfonyl)-3,4-dihydro-2(1H)-pyrazinone;
2-((2R)-1-((4-methylphenyl)sulfonyl)-3-oxo-1,2,3,4-tetrahydro-2-pyrazinyl)-N-((1R,2R,4R)-1,7,7-trimethylbicyclo[2.2.1]hept-2-yl)acetamide; and 2-((2R)-1-((4-methylphenyl)sulfonyl)-3-oxo-1,2,3,4-tetrahydro-2-pyrazinyl)-N-((1R,2S,4R)-1,7,7-trimethylbicyclo[2.2.1]hept-2-yl)acetamide;
2-((2R)-1-((4-methylphenyl)sulfonyl)-3-oxo-1,2,3,4-tetrahydro-2-pyrazinyl)-N-(1-phenyl-4-piperidinyl)acetamide;
N-((1S,2S)-2-(1,1-dimethylethyl)cyclohexyl)-2-((2R)-1-((4-methylphenyl)-sulfonyl)-3-oxo-1,2,3,4-tetrahydro-2-pyrazinyl)acetamide; N-((1R,2R)-2-(1,1-dimethylethyl)cyclohexyl)-2-((2R)-1-((4-methylphenyl)-sulfonyl)-3-oxo-1,2,3,4-tetrahydro-2-pyrazinyl)acetamide; N-((1S,2R)-2-(1,1-dimethylethyl)cyclohexyl)-2-((2R)-1-((4-methylphenyl)-sulfonyl)-3-oxo-1,2,3,4-tetrahydro-2-pyrazinyl)acetamide; and N-((1R,2S)-2-(1,1-dimethylethyl)cyclohexyl)-2-((2R)-1-((4-methylphenyl)-sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-((3S)-1-(phenylmethyl)-3-pyrrolidinyl)acetamide; and 2-((2R)-1-((4-methyl-phenyl)sulfonyl)-3-oxo-1,2,3,4-tetrahydro-2-pyrazinyl)-N-((3R)-1-(phenylmethyl)-3-pyrrolidinyl)acetamide;
N-((3R)-1-azabicyclo[2.2.2]oct-3-yl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(2-pyrimidinyl)-4-piperidinyl)acetamide;
1,1-dimethylethyl (3R)-3-((((2R)-1-((4-methylphenyl)sulfonyl)-3-oxo-1,2,3,4-tetrahydro-2-pyrazinyl)acetyl)amino)-1-piperidinecarboxylate; and 1,1-dimethylethyl (3S)-3-((((2R)-1-((4-methylphenyl)sulfonyl)-3-oxo-1,2,3,4-tetrahydro-2-pyrazinyl)-acetyl)amino)-1-piperidinecarboxylate;
2-((2R)-1-((4-methylphenyl)sulfonyl)-3-oxo-1,2,3,4-tetrahydro-2-pyrazinyl)-N-(1-(4-pyridinyl)-4-piperidinyl)acetamide;
2-((2R)-1-((4-methylphenyl)sulfonyl)-3-oxo-1,2,3,4-tetrahydro-2-pyrazinyl)-N-((3R)-1-(phenylmethyl)-3-piperidinyl)acetamide; and 2-((2R)-1-((4-methylphenyl)-sulfonyl)-3-oxo-1,2,3,4-tetrahydro-2-pyrazinyl)-N-((3S)-1-(phenylmethyl)-3-piperidinyl)acetamide;
N-((3R)-1-(1-methylethyl)-3-piperidinyl)-2-((2R)-1-((4-methyl-phenyl)sulfonyl)-3-oxo-1,2,3,4-tetrahydro-2-pyrazinyl)acetamide; and N-((3S)-1-(1-methylethyl)-3-piperidinyl)-2-((2R)-1-((4-methylphenyl)sulfonyl)-3-oxo-1,2,3,4-tetrahydro-2-pyrazinyl)acetamide;
1,1-dimethylethyl 4-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(1-(4-pyridinylmethyl)-4-piperidinyl)acetamide;
2-((2R)-1-((4-methylphenyl)sulfonyl)-3-oxo-1,2,3,4-tetrahydro-2-pyrazinyl)-N-((3S)-1-(4-pyridinylmethyl)-3-piperidinyl)acetamide; and 2-((2R)-1-((4-methylphenyl)sulfonyl)-3-oxo-1,2,3,4-tetrahydro-2-pyrazinyl)-N-((3R)-1-(4-pyridinylmethyl)-3-piperidinyl)acetamide;
1,1-dimethylethyl (4R)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate; and 1,1-dimethylethyl (4S)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(1-methyl-4-piperidinyl)acetamide;
N-((4S)-3,3-dimethyl-4-piperidinyl)-2-((2R)-1-((4-methylphenyl)sulfonyl)-3-oxo-1,2,3,4-tetrahydro-2-pyrazinyl)acetamide; and N-((4R)-3,3-dimethyl-4-piperidinyl)-2-((2R)-1-((4-methylphenyl)sulfonyl)-3-oxo-1,2,3,4-tetrahydro-2-pyrazinyl)acetamide;
N-(1-acetyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4S)-3,3-dimethyl-1-(2-methylpropyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-methylpropyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(phenylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(phenylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1,3,3-trimethyl-4-piperidinyl)acetamide;
N-((4R)-3,3-dimethyl-1-(1-methylethyl)-4-piperidinyl)-2-((2R)-1-((4-methyl-phenyl)sulfonyl)-3-oxo-1,2,3,4-tetrahydro-2-pyrazinyl)acetamide; and N-((4S)-3,3-dimethyl-1-(1-methylethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(4-pyridinylmethyl)-4-piperidinyl)-2-((2R)-1-((4-methylphenyl)sulfonyl)-3-oxo-1,2,3,4-tetrahydro-2-pyrazinyl)acetamide; and N-((4S)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(phenylcarbonyl)-4-piperidinyl)acetamide;
N-((4S)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide; and N-((4R)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-(trans-4-(methyloxy)cyclohexyl)-2-((2R)-1-((4-methylphenyl)sulfonyl)-3-oxo-1,2,3,4-tetrahydro-2-pyrazinyl)acetamide;
N-(cis-4-aminocyclohexyl)-2-((2R)-1-((4-methylphenyl)sulfonyl)-3-oxo-1,2,3,4-tetrahydro-2-pyrazinyl)acetamide;
N-(trans-4-aminocyclohexyl)-2-((2R)-1-((4-methylphenyl)sulfonyl)-3-oxo-1,2,3,4-tetrahydro-2-pyrazinyl)acetamide;
N-(trans-4-aminocyclohexyl)-2-((2R)-1-((2,3-dichlorophenyl)sulfonyl)-3-oxo-1,2,3,4-tetrahydro-2-pyrazinyl)acetamide;
N-((3-endo)-8-methyl-8-azabicyclo[3.2.1]oct-3-yl)-2-((2R)-1-((4-methylphenyl)-sulfonyl)-3-oxo-1,2,3,4-tetrahydro-2-pyrazinyl)acetamide; and N-((3-exo)-8-methyl-8-azabicyclo[3.2.1]oct-3-yl)-2-((2R)-1-((4-methylphenyl)-sulfonyl)-3-oxo-1,2,3,4-tetrahydro-2-pyrazinyl)acetamide;
N-(cis-4-((1-methylethyl)amino)cyclohexyl)-2-((2R)-1-((4-methyl-phenyl)sulfonyl)-3-oxo-1,2,3,4-tetrahydro-2-pyrazinyl)acetamide;
N-(trans-4-((1-methylethyl)amino)cyclohexyl)-2-((2R)-1-((4-methylphenyl)-sulfonyl)-3-oxo-1,2,3,4-tetrahydro-2-pyrazinyl)acetamide;
N-((1R)-2,2-dimethyl-4-oxocyclohexyl)-2-((2R)-1-((4-methyl-phenyl)sulfonyl)-3-oxo-1,2,3,4-tetrahydro-2-pyrazinyl)acetamide; and N-((1S)-2,2-dimethyl-4-oxocyclohexyl)-2-((2R)-1-((4-methylphenyl)sulfonyl)-3-oxo-1,2,3,4-tetrahydro-2-pyrazinyl)acetamide;
N-(2,3-dimethylcyclohexyl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(4-methyl-1-piperazinyl)acetamide;
N-1,1′-bi(cyclohexyl)-2-yl-2-((2R)-1-((4-methylphenyl)sulfonyl)-3-oxo-1,2,3,4-tetrahydro-2-pyrazinyl)acetamide;
N-(4-(3-fluoro-1-piperidinyl)-2,2-dimethylcyclohexyl)-2-((2R)-1-((4-methyl-phenyl)sulfonyl)-3-oxo-1,2,3,4-tetrahydro-2-pyrazinyl)acetamide;
N-((1R,4S)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1R,4R)-4-(4-fluoro-1-piperidinyl)-2,2-dimethylcyclohexyl)-2-((2R)-1-((4-methylphenyl)-sulfonyl)-3-oxo-1,2,3,4-tetrahydro-2-pyrazinyl)acetamide; N-((1S,4S)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-(4-fluoro-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide;
N-((1S,4R)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide;
N-((1S,4S)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide; N-((1R,4R)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide; and N-((1R,4S)-4-(cyclopropylamino)-2,2-dimethylcyclohexyl)-2-((2R)-1-((4-methyl-phenyl)sulfonyl)-3-oxo-1,2,3,4-tetrahydro-2-pyrazinyl)acetamide;
N-((1R,4S)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; N-(((1R,4R)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; N-((1S,4S)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-((2,2-dimethylpropyl)amino)-2,2-dimethylcyclohexyl)-2-((2R)-1-((4-methylphenyl)sulfonyl)-3-oxo-1,2,3,4-tetrahydro-2-pyrazinyl)acetamide;
N-((4S)-1-cyclopropyl-3,3-dimethyl-4-piperidinyl)-2-((2R)-1-((4-methylphenyl)-sulfonyl)-3-oxo-1,2,3,4-tetrahydro-2-pyrazinyl)acetamide; and N-((4R)-1-cyclopropyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4R)-3,3-dimethyl-1-(2,2,2-trifluoroethyl)-4-piperidinyl)-2-((2R)-1-((4-methyl-phenyl)sulfonyl)-3-oxo-1,2,3,4-tetrahydro-2-pyrazinyl)acetamide; and N-((4S)-3,3-dimethyl-1-(2,2,2-trifluoroethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-((4S)-3,3-dimethyl-4-piperidinyl)acetamide; and 2-((2R)-1-((2,3-dichlorophenyl)sulfonyl)-3-oxo-1,2,3,4-tetrahydro-2-pyrazinyl)-N-((4R)-3,3-dimethyl-4-piperidinyl)acetamide;
2-((2R)-1-((4-chloro-2,5-dimethylphenyl)sulfonyl)-3-oxo-1,2,3,4-tetrahydro-2-pyrazinyl)-N-((4S)-3,3-dimethyl-4-piperidinyl)acetamide; and 2-((2R)-1-((4-chloro-2,5-dimethylphenyl)sulfonyl)-3-oxo-1,2,3,4-tetrahydro-2-pyrazinyl)-N-((4R)-3,3-dimethyl-4-piperidinyl)acetamide;
2-((2R)-1-((3,4-dichlorophenyl)sulfonyl)-3-oxo-1,2,3,4-tetrahydro-2-pyrazinyl)-N-((4R)-3,3-dimethyl-4-piperidinyl)acetamide; and 2-((2R)-1-((3,4-dichlorophenyl)sulfonyl)-3-oxo-1,2,3,4-tetrahydro-2-pyrazinyl)-N-((4S)-3,3-dimethyl-4-piperidinyl)acetamide;
N-((1R,4S)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide; and N-((1S,4R)-2,2-dimethyl-4-(1-pyrrolidinyl)cyclohexyl)-2-((2R)-1-((4-methyl-phenyl)sulfonyl)-3-oxo-1,2,3,4-tetrahydro-2-pyrazinyl)acetamide;
N-((1R,4S)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(1-piperidinyl)-cyclohexyl)-2-((2R)-1-((4-methyl-phenyl)sulfonyl)-3-oxo-1,2,3,4-tetrahydro-2-pyrazinyl)acetamide; and N-((1S,4R)-2,2-dimethyl-4-(1-piperidinyl)cyclohexyl)-2-((2R)-1-((4-methyl-phenyl)sulfonyl)-3-oxo-1,2,3,4-tetrahydro-2-pyrazinyl)acetamide;
N-((1S,4R)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; N-((1S,4S)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; N-((1R,4R)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide; and N-((1R,4S)-2,2-dimethyl-4-(4-methyl-1-piperidinyl)cyclohexyl)-2-((2R)-1-((4-methylphenyl)sulfonyl)-3-oxo-1,2,3,4-tetrahydro-2-pyrazinyl)acetamide;
N-((1R,4S)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1R,4R)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; N-((1S,4S)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide; and N-((1S,4R)-4-(3,3-dimethyl-1-piperidinyl)-2,2-dimethylcyclohexyl)-2-((2R)-1-((4-methylphenyl)sulfonyl)-3-oxo-1,2,3,4-tetrahydro-2-pyrazinyl)acetamide;
N-((1S,4R)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; N-((1S,4S)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; N-((1R,4R)-2,2-dimethyl-4-(4-morpholinyl)cyclohexyl)-2-((2R)-1-((4-methyl-phenyl)sulfonyl)-3-oxo-1,2,3,4-tetrahydro-2-pyrazinyl)acetamide; and N-((1R,4S)-2,2-dimethyl-4-(4-morpholinyl)-cyclohexyl)-2-((2R)-1-((4-methyl-phenyl)sulfonyl)-3-oxo-1,2,3,4-tetrahydro-2-pyrazinyl)acetamide;
N-((1S,4R)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; N-((1S,4S)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; N-((1R,4R)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide; and N-((1R,4S)-4-hydroxy-2,2-dimethylcyclohexyl)-2-((2R)-1-((4-methylphenyl)-sulfonyl)-3-oxo-1,2,3,4-tetrahydro-2-pyrazinyl)acetamide;
N-((4S)-3,3-dimethyl-4-piperidinyl)-2-((2R)-1-((4-methylphenyl)sulfonyl)-3-oxo-1,2,3,4-tetrahydro-2-pyrazinyl)acetamide; 123
N-((4R)-3,3-dimethyl-4-piperidinyl)-2-((2R)-1-((4-methylphenyl)sulfonyl)-3-oxo-1,2,3,4-tetrahydro-2-pyrazinyl)acetamide;
N-(1-(2-phenethyl)-4-piperidinyl)-2-((2R)-1-((4-methylphenyl)sulfonyl)-3-oxo-1,2,3,4-tetrahydro-2-pyrazinyl)acetamide;
N-((4S)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-((4R)-3,3-dimethyltetrahydro-2H-pyran-4-yl)-2-((2R)-1-((4-methylphenyl)-sulfonyl)-3-oxo-1,2,3,4-tetrahydro-2-pyrazinyl)acetamide;
N-((3R)-3-piperidinyl)-2-((2R)-1-((4-methylphenyl)sulfonyl)-3-oxo-1,2,3,4-tetrahydro-2-pyrazinyl)acetamide; and N-((3S)-3-piperidinyl)-2-((2R)-1-((4-methylphenyl)sulfonyl)-3-oxo-1,2,3,4-tetrahydro-2-pyrazinyl)acetamide;
1,1-dimethylethyl 4-methyl-4-((((2R)-1-((4-methylphenyl)sulfonyl)-3-oxo-1,2,3,4-tetrahydro-2-pyrazinyl)acetyl)amino)-1-piperidinecarboxylate;
N-(4-methyl-4-piperidinyl)-2-((2R)-1-((4-methylphenyl)sulfonyl)-3-oxo-1,2,3,4-tetrahydro-2-pyrazinyl)acetamide;
N-(3-(hydroxymethyl)bicyclo[2.2.1]hept-2-yl)-2-((2R)-1-((4-methylphenyl)sulfonyl)-3-oxo-1,2,3,4-tetrahydro-2-pyrazinyl)acetamide;
N-((1RS,2RS)-2-(1-hydroxy-1-methylethyl)cyclohexyl)-2-((2R)-1-((4-methylphenyl)sulfonyl)-3-oxo-1,2,3,4-tetrahydro-2-pyrazinyl)acetamide;
1,1-dimethylethyl 4-((((2R)-1-((4-methylphenyl)sulfonyl)-3-oxo-1,2,3,4-tetrahydro-2-pyrazinyl)acetyl)amino)-1-piperidinecarboxylate;
N-((4RS)-2,2-dimethyl-6-oxotetrahydro-2H-pyran-4-yl)-2-((2R)-1-((4-methylphenyl)sulfonyl)-3-oxo-1,2,3,4-tetrahydro-2-pyrazinyl)acetamide;
N-((4RS)-3,3-dimethyl-1,4′-bipiperidin-4-yl)-2-((2R)-1-((4-methylphenyl)-sulfonyl)-3-oxo-1,2,3,4-tetrahydro-2-pyrazinyl)acetamide;
N-((1RS,2RS)-2-(hydroxymethyl)cyclohexyl)-2-((2R)-1-((4-methylphenyl)-sulfonyl)-3-oxo-1,2,3,4-tetrahydro-2-pyrazinyl)acetamide;
1,1-dimethylethyl (3RS)-3-((((2R)-1-((4-methylphenyl)sulfonyl)-3-oxo-1,2,3,4-tetrahydro-2-pyrazinyl)acetyl)amino)-1-piperidinecarboxylate;
2-((2R)-1-((4-methylphenyl)sulfonyl)-3-oxo-1,2,3,4-tetrahydro-2-pyrazinyl)-N-(tetrahydro-2H-pyran-4-yl)acetamide;
N-((4R)-3,3-dimethyl-1-(2-methylpropyl)-4-piperidinyl)-2-((2R)-1-((4-methylphenyl)sulfonyl)-3-oxo-1,2,3,4-tetrahydro-2-pyrazinyl)acetamide;
N-((4R/S)-4-azepanyl)-2-((2R)-1-((4-chloro-2,5-dimethylphenyl)sulfonyl)-3-oxo-1,2,3,4-tetrahydro-2-pyrazinyl)acetamide trifluoroacetic acid salt;
N-((4R)-4-azepanyl)-2-((2R)-1-((4-methylphenyl)sulfonyl)-3-oxo-1,2,3,4-tetrahydro-2-pyrazinyl)acetamide and N-((4S)-4-azepanyl)-2-((2R)-1-((4-methyl-phenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-((3R)-3-pyrrolidinyl)acetamide;
N-((1R/S,4R/S)-4-hydroxy-2,2,4-trimethylcyclohexyl)-2-((2R)-1-((4-methylphenyl)sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1,2,2,6,6-pentamethyl-4-piperidinyl)acetamide;
N-((4R/S)-1-methyl-4-azepanyl)-2-((2R)-1-((4-methylphenyl)sulfonyl)-3-oxo-1,2,3,4-tetrahydro-2-pyrazinyl)acetamide;
N-(trans-4-(dimethylamino)cyclohexyl)-2-((2R)-1-((4-methylphenyl)-sulfonyl)-3-oxo-1,2,3,4-tetrahydro-2-pyrazinyl)acetamide;
N-(cis/trans-5-aminocyclooctyl)-2-((2R)-1-((2,3-dichlorophenyl)sulfonyl)-3-oxo-1,2,3,4-tetrahydro-2-pyrazinyl)acetamide;
N-((4R)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(4-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(3-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4S)-3,3-dimethyl-1-(3-pyridinylmethyl)-4-piperidinyl)-2-((2R)-1-((4-methyl-phenyl)sulfonyl)-3-oxo-1,2,3,4-tetrahydro-2-pyrazinyl)acetamide;
2-((2R)-1-((2,3-dichlorophenyl)sulfonyl)-3-oxo-1,2,3,4-tetrahydro-2-pyrazinyl)-N-(4-methyl-1-piperazinyl)acetamide 3,3,3-trifluoropropanoic acid salt;
N-(4,4-difluorocyclohexyl)-2-((2R)-1-((4-methylphenyl)sulfonyl)-3-oxo-1,2,3,4-tetrahydro-2-pyrazinyl)acetamide;
2-((2R)-1-((4-chloro-2,5-dimethylphenyl)sulfonyl)-3-oxo-1,2,3,4-tetrahydro-2-pyrazinyl)-N-(4-methyl-1-piperazinyl)acetamide;
1,1-dimethylethyl (4S)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
1,1-dimethylethyl (4R)-3,3-dimethyl-4-((((2R)-1-((4-methylphenyl)sulfonyl)-3-oxo-1,2,3,4-tetrahydro-2-pyrazinyl)acetyl)amino)-1-piperidinecarboxylate;
N-(1-cyclopropyl-4-piperidinyl)-2-((2R)-1-((4-methylphenyl)sulfonyl)-3-oxo-1,2,3,4-tetrahydro-2-pyrazinyl)acetamide;
N-((4R/S)-1-cyclopropyl-4-azepanyl)-2-((2R)-1-((4-methylphenyl)sulfonyl)-3-oxo-1,2,3,4-tetrahydro-2-pyrazinyl)acetamide;
2-((2R)-1-((4-chloro-2,5-dimethylphenyl)sulfonyl)-3-oxo-1,2,3,4-tetrahydro-2-pyrazinyl)-N-((4R/S)-1-cyclopropyl-4-azepanyl)acetamide trifluoroacetic acid salt;
2-((2R)-1-((4-chloro-2,5-dimethylphenyl)sulfonyl)-3-oxo-1,2,3,4-tetrahydro-2-pyrazinyl)-N-((4S)-3,3-dimethyl-4-piperidinyl)acetamide;
2-((2R)-1-((4-methylphenyl)sulfonyl)-3-oxo-1,2,3,4-tetrahydro-2-pyrazinyl)-N-((4S)-1,3,3-trimethyl-4-piperidinyl)acetamide;
N-((4S)-1-ethyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4S)-3,3-dimethyl-1-propyl-4-piperidinyl)-2-((2R)-1-((4-methylphenyl)-sulfonyl)-3-oxo-1,2,3,4-tetrahydro-2-pyrazinyl)acetamide;
N-((4S)-1-(2,2-dimethylpropyl)-3,3-dimethyl-4-piperidinyl)-2-((2R)-1-((4-methylphenyl)sulfonyl)-3-oxo-1,2,3,4-tetrahydro-2-pyrazinyl)acetamide;
N-((4S)-1-cyclobutyl-3,3-dimethyl-4-piperidinyl)-2-((2R)-1-((4-methylphenyl)-sulfonyl)-3-oxo-1,2,3,4-tetrahydro-2-pyrazinyl)acetamide;
N-((4S)-1-cyclopentyl-3,3-dimethyl-4-piperidinyl)-2-((2R)-1-((4-methylphenyl)-sulfonyl)-3-oxo-1,2,3,4-tetrahydro-2-pyrazinyl)acetamide;
1,1-dimethylethyl (4S)-4-((((2R)-1-((2,3-dichlorophenyl)sulfonyl)-3-oxo-1,2,3,4-tetrahydro-2-pyrazinyl)acetyl)amino)-3,3-dimethyl-1-piperidinecarboxylate;
2-((2R)-1-((4-chloro-2,5-dimethylphenyl)sulfonyl)-3-oxo-1,2,3,4-tetrahydro-2-pyrazinyl)-N-((4S)-1,3,3-trimethyl-4-piperidinyl)acetamide;
2-((2R)-1-((2,3-dichlorophenyl)sulfonyl)-3-oxo-1,2,3,4-tetrahydro-2-pyrazinyl)-N-((4S)-3,3-dimethyl-4-piperidinyl)acetamide;
2-((2R)-1-((2,3-dichlorophenyl)sulfonyl)-3-oxo-1,2,3,4-tetrahydro-2-pyrazinyl)-N-((4S)-1,3,3-trimethyl-4-piperidinyl)acetamide;
N-((4S)-1-(2,2-dimethylpropanoyl)-3,3-dimethyl-4-piperidinyl)-2-((2R)-1-((4-methylphenyl)sulfonyl)-3-oxo-1,2,3,4-tetrahydro-2-pyrazinyl)acetamide;
N-5-azocanyl-2-((2R)-1-((4-methylphenyl)sulfonyl)-3-oxo-1,2,3,4-tetrahydro-2-pyrazinyl)acetamide;
1,1-dimethylethyl (4S)-4-((((2R)-1-((4-chloro-2,5-dimethylphenyl)sulfonyl)-3-oxo-1,2,3,4-tetrahydro-2-pyrazinyl)acetyl)amino)-3,3-dimethyl-1-piperidinecarboxylate;
N-((1RS,2RS)-2-hydroxy-2-methylcyclohexyl)-2-((2R)-1-((4-methylphenyl)-sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-(1-(4-pyridinyl)-4-piperidinyl)acetamide;
2-((2R)-1-((4-methylphenyl)sulfonyl)-3-oxo-1,2,3,4-tetrahydro-2-pyrazinyl)-N-(1-(4-pyridinylmethyl)-4-piperidinyl)acetamide;
2-((2R)-1-((4-methylphenyl)sulfonyl)-3-oxo-1,2,3,4-tetrahydro-2-pyrazinyl)-N-1-piperidinylacetamide;
N-((8RS)-7,7-dimethyl-1,4-dioxaspiro[4.5]dec-8-yl)-2-((2R)-1-((4-methyl-phenyl)-sulfonyl)-3-oxo-1,2,3,4-tetrahydro-2-pyrazinyl)acetamide;
2-((2R)-1-((4-methylphenyl)sulfonyl)-3-oxo-1,2,3,4-tetrahydro-2-pyrazinyl)-N-((3S)-6-oxo-3-piperidinyl)acetamide;
2-((2R)-1-((4-methylphenyl)sulfonyl)-3-oxo-1,2,3,4-tetrahydro-2-pyrazinyl)-N-((3R)-6-oxo-3-piperidinyl)acetamide;
1,1-dimethylethyl (4RS)-4-((((2R)-1-((4-chloro-2,5-dimethylphenyl)sulfonyl)-3-oxo-1,2,3,4-tetrahydro-2-pyrazinyl)acetyl)amino)-1-azepanecarboxylate;
N-((4S)-3,3-dimethyl-1,4′-bipiperidin-4-yl)-2-((2R)-1-((4-methylphenyl)-sulfonyl)-3-oxo-1,2,3,4-tetrahydro-2-pyrazinyl)acetamide;
N-((4R)-3,3-dimethyl-1,4′-bipiperidin-4-yl)-2-((2R)-1-((4-methylphenyl)-sulfonyl)-3-oxo-1,2,3,4-tetrahydro-2-pyrazinyl)acetamide;
N-((4R)-1-cyclopropyl-3,3-dimethyl-4-piperidinyl)-2-((2R)-1-((2,3-dichloro-phenyl)sulfonyl)-3-oxo-1,2,3,4-tetrahydro-2-pyrazinyl)acetamide;
N-((4S)-1-cyclopropyl-3,3-dimethyl-4-piperidinyl)-2-((2R)-1-((2,3-dichloro-phenyl)sulfonyl)-3-oxo-1,2,3,4-tetrahydro-2-pyrazinyl)acetamide;
N-((4S)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-pyridinylmethyl)-4-piperidinyl)-2-((2R)-1-((4-methyl-phenyl)sulfonyl)-3-oxo-1,2,3,4-tetrahydro-2-pyrazinyl)acetamide;
N-((4R)-3,3-dimethyl-1-(2-(methyloxy)ethyl)-4-piperidinyl)-2-((2R)-1-((4-methylphenyl)sulfonyl)-3-oxo-1,2,3,4-tetrahydro-2-pyrazinyl)acetamide;
N-((4S) and 4(R)-1-cyclopropyl-3,3-dimethyl-4-piperidinyl)-2-((2R)-1-((4-methylphenyl)sulfonyl)-3-oxo-1,2,3,4-tetrahydro-2-pyrazinyl)acetamide n-oxide;
2-((2R)-1-((4-methylphenyl)sulfonyl)-3-oxo-1,2,3,4-tetrahydro-2-pyrazinyl)-N-methyl-N-(1-methyl-4-piperidinyl)acetamide;
2-((2R)-1-((2,3-dichlorophenyl)sulfonyl)-3-oxo-1,2,3,4-tetrahydro-2-pyrazinyl)-N-methyl-N-(1-methyl-4-piperidinyl)acetamide;
2-((2R)-1-((3,4-dichlorophenyl)sulfonyl)-3-oxo-1,2,3,4-tetrahydro-2-pyrazinyl)-N-methyl-N-(1-methyl-4-piperidinyl)acetamide;
2-((2R)-1-((4-chloro-2,5-dimethylphenyl)sulfonyl)-3-oxo-1,2,3,4-tetrahydro-2-pyrazinyl)-N-methyl-N-(1-methyl-4-piperidinyl)acetamide;
N-(cis-4-aminocyclohexyl)-N-methyl-2-((2R)-1-((4-methylphenyl)sulfonyl)-3-oxo-1,2,3,4-tetrahydro-2-pyrazinyl)acetamide; and N-(trans-4-aminocyclohexyl)-N-methyl-2-((2R)-1-((4-methylphenyl)sulfonyl)-3-oxo-1,2,3,4-tetrahydro-2-pyrazinyl)-acetamide;
N-((4R)-3,3-dimethyl-4-piperidinyl)-N-methyl-2-((2R)-1-((4-methylphenyl)-sulfonyl)-3-oxo-1,2,3,4-tetrahydro-2-pyrazinyl)acetamide; and N-((4S)-3,3-dimethyl-4-piperidinyl)-N-methyl-2-((2R)-1-((4-methylphenyl)-sulfonyl)-3-oxo-1,2,3,4-tetrahydro-2-pyrazinyl)acetamide;
N-((4RS)-1-cyclopropyl-3,3-dimethyl-4-piperidinyl)-N-methyl-2-((2R)-1-((4-methylphenyl)sulfonyl)-3-oxo-1,2,3,4-tetrahydro-2-pyrazinyl)acetamide;
1,1-dimethylethyl 4-(ethyl(((2R)-1-((4-methylphenyl)sulfonyl)-3-oxo-1,2,3,4-tetrahydro-2-pyrazinyl)acetyl)amino)-1-piperidinecarboxylate;
N-methyl-2-((2R)-1-((4-methylphenyl)sulfonyl)-3-oxo-1,2,3,4-tetrahydro-2-pyrazinyl)-N-4-piperidinylacetamide;
N-ethyl-2-((2R)-1-((4-methylphenyl)sulfonyl)-3-oxo-1,2,3,4-tetrahydro-2-pyrazinyl)-N-4-piperidinylacetamide;
N-propyl-2-((2R)-1-((4-methylphenyl)sulfonyl)-3-oxo-1,2,3,4-tetrahydro-2-pyrazinyl)-N-4-piperidinylacetamide;
2-((2R)-1-((4-chloro-2,5-dimethylphenyl)sulfonyl)-3-oxo-1,2,3,4-tetrahydro-2-pyrazinyl)-N-methyl-N-(1-methyl-4-piperidinyl)acetamide;
1,1-dimethylethyl 4-(methyl(((2R)-1-((4-methylphenyl)sulfonyl)-3-oxo-1,2,3,4-tetrahydro-2-pyrazinyl)acetyl)amino)-1-piperidinecarboxylate;
1,1-dimethylethyl 4-((((2R)-1-((4-methylphenyl)sulfonyl)-3-oxo-1,2,3,4-tetrahydro-2-pyrazinyl)acetyl)(propyl)amino)-1-piperidinecarboxylate;
N-(2-(methyloxy)ethyl)-2-((2R)-1-((4-methylphenyl)sulfonyl)-3-oxo-1,2,3,4-tetrahydro-2-pyrazinyl)-N-(1-methyl-4-piperidinyl)acetamide;
1,1-dimethylethyl (4RS)-4-(methyl(((2R)-1-((4-methylphenyl)sulfonyl)-3-oxo-1,2,3,4-tetrahydro-2-pyrazinyl)acetyl)amino)-1-azepanecarboxylate;
1,1-dimethylethyl (4R/S)-4-(ethyl(((2R)-1-((4-methylphenyl)sulfonyl)-3-oxo-1,2,3,4-tetrahydro-2-pyrazinyl)acetyl)amino)-1-azepanecarboxylate;
N-((4R/S)-4-azepanyl)-N-methyl-2-((2R)-1-((4-methylphenyl)sulfonyl)-3-oxo-1,2,3,4-tetrahydro-2-pyrazinyl)acetamide;
N-((4R/S)-4-azepanyl)-N-ethyl-2-((2R)-1-((4-methylphenyl)sulfonyl)-3-oxo-1,2,3,4-tetrahydro-2-pyrazinyl)acetamide;
1,1-dimethylethyl 4-(cyclopropyl(((2R)-1-((4-methylphenyl)sulfonyl)-3-oxo-1,2,3,4-tetrahydro-2-pyrazinyl)acetyl)amino)-1-piperidinecarboxylate;
N-cyclopropyl-2-((2R)-1-((4-methylphenyl)sulfonyl)-3-oxo-1,2,3,4-tetrahydro-2-pyrazinyl)-N-4-piperidinylacetamide;
N-methyl-2-((2R)-1-((4-methylphenyl)sulfonyl)-3-oxo-1,2,3,4-tetrahydro-2-pyrazinyl)-N-(1-methyl-4-piperidinyl)acetamide;
(3R)-3-(2-(2-(4-(methyloxy)phenyl)-4-thiomorpholinyl)-2-oxoethyl)-4-((4-methyl-phenyl)sulfonyl)-3,4-dihydro-2(1H)-pyrazinone;
(3R)-3-(2-(2-(4-chlorophenyl)-1-pyrrolidinyl)-2-oxoethyl)-4-((4-methylphenyl)-sulfonyl)-3,4-dihydro-2(1H)-pyrazinone;
(3R)-4-((2,3-dichlorophenyl)sulfonyl)-3-(2-((3S)-3-(dimethylamino)-1-pyrrolidinyl)-2-oxoethyl)-3,4-dihydro-2(1H)-pyrazinone;
(3R)-4-((2,3-dichlorophenyl)sulfonyl)-3-(2-((3R)-3-(dimethylamino)-1-pyrrolidinyl)-2-oxoethyl)-3,4-dihydro-2(1H)-pyrazinone;
(3R)-3-(2-((3S)-3-(dimethylamino)-1-pyrrolidinyl)-2-oxoethyl)-4-((4-methylphenyl)sulfonyl)-3,4-dihydro-2(1H)-pyrazinone;
(3R)-4-((4-chloro-2,5-dimethylphenyl)sulfonyl)-3-(2-((3R)-3-(dimethylamino)-1-pyrrolidinyl)-2-oxoethyl)-3,4-dihydro-2(1H)-pyrazinone;
(3R)-3-(2-(1,4′-bipiperidin-1′-yl)-2-oxoethyl)-4-((4-chloro-2,5-dimethylphenyl)-sulfonyl)-3,4-dihydro-2(1H)-pyrazinone;
(3R)-4-((4-chloro-2,5-dimethylphenyl)sulfonyl)-3-(2-(4-(dimethylamino)-1-piperidinyl)-2-oxoethyl)-3,4-dihydro-2(1H)-pyrazinone;
(3R)-3-(2-((3S)-3-(amino)-4(R)-4-phenyl-1-pyrrolidinyl)-2-oxoethyl)-4-((4-methylphenyl)sulfonyl)-3,4-dihydro-2(1H)-pyrazinone;
(3R)-4-((4-chloro-2,5-dimethylphenyl)sulfonyl)-3-(2-oxo-2-((3S)-3-(1-pyrrolidinyl)-1-piperidinyl)ethyl)-3,4-dihydro-2(1H)-pyrazinone;
(3R)-3-(2-((3′S)-1,3′-bipyrrolidin-1′-yl)-2-oxoethyl)-4-((4-chloro-2,5-dimethylphenyl)sulfonyl)-3,4-dihydro-2(1H)-pyrazinone;
(3R)-4-((4-chloro-2,5-dimethylphenyl)sulfonyl)-3-(2-((3S)-3-(dimethylamino)-1-piperidinyl)-2-oxoethyl)-3,4-dihydro-2(1H)-pyrazinone;
(3R)-4-((4-chloro-2,5-dimethylphenyl)sulfonyl)-3-(2-((3S)-3-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-(4-(hydroxymethyl)-1-piperidinyl)-2-oxoethyl)-3,4-dihydro-2(1H)-pyrazinone;
(3R/S)-4-((4-chloro-2,5-dimethylphenyl)sulfonyl)-3-(2-((3R)-3-(hydroxymethyl)-1-piperidinyl)-2-oxoethyl)-3,4-dihydro-2(1H)-pyrazinone;
(3R)-4-((4-chloro-2,5-dimethylphenyl)sulfonyl)-3-(2-((2R/S)-2-(hydroxymethyl)-1-piperidinyl)-2-oxoethyl)-3,4-dihydro-2(1H)-pyrazinone;
(3R)-4-((4-chloro-2,5-dimethylphenyl)sulfonyl)-3-(2-oxo-2-(4-(1-pyrrolidinylmethyl)-1-piperidinyl)ethyl)-3,4-dihydro-2(1H)-pyrazinone;
(3R)-4-((4-chloro-2,5-dimethylphenyl)sulfonyl)-3-(2-oxo-2-((3R/S)-3-(1-pyrrolidinylmethyl)-1-piperidinyl)ethyl)-3,4-dihydro-2(1H)-pyrazinone;
(3R)-3-(2-((3S)-3-amino-1-pyrrolidinyl)-2-oxoethyl)-4-((4-chloro-2,5-dimethyl-phenyl)sulfonyl)-3,4-dihydro-2(1H)-pyrazinone;
(3R)-4-((4-chloro-2,5-dimethylphenyl)sulfonyl)-3-(2-((4R/S)-4-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-((3R/S)-3-((dimethylamino)-methyl)-1-piperidinyl)-2-oxoethyl)-3,4-dihydro-2(1H)-pyrazinone;
(3R)-4-((4-chloro-2,5-dimethylphenyl)sulfonyl)-3-(2-oxo-2-((3S)-3-(1-pyrrolidinyl)-1-azepanyl)ethyl)-3,4-dihydro-2(1H)-pyrazinone;
(3R)-4-((4-chloro-2,5-dimethylphenyl)sulfonyl)-3-(2-((3′R/S,4′R/S)-4′-methyl-1,3′-bipyrrolidin-1′-yl)-2-oxoethyl)-3,4-dihydro-2(1H)-pyrazinone;
(3R)-4-((4-chloro-2,5-dimethylphenyl)sulfonyl)-3-(2-oxo-2-((3R/S)-3-(1-pyrrolidinyl-methyl)-1-pyrrolidinyl)ethyl)-3,4-dihydro-2(1H)-pyrazinone;
(3R)-4-((4-chloro-2,5-dimethylphenyl)sulfonyl)-3-(2-(2,7-diazaspiro[4.4]non-2-yl)-2-oxoethyl)-3,4-dihydro-2(1H)-pyrazinone;
(3R)-4-((2,3-dichlorophenyl)sulfonyl)-3-(2-((4R/S)-4-(dimethylamino)-1-azepanyl)-2-oxoethyl)-3,4-dihydro-2(1H)-pyrazinone;
(3R)-4-((4-chloro-2,5-dimethylphenyl)sulfonyl)-3-(2-((4R/S)-4-(cyclopropyl(methyl)amino)-1-azepanyl)-2-oxoethyl)-3,4-dihydro-2(1H)-pyrazinone trifluoroacetic acid salt;
(3R)-4-((4-chloro-2,5-dimethylphenyl)sulfonyl)-3-(2-((4R/S)-4-hydroxy-3,3-dimethyl-1-piperidinyl)-2-oxoethyl)-3,4-dihydro-2(1H)-pyrazinone;
(3R)-4-((4-chloro-2,5-dimethylphenyl)sulfonyl)-3-(2-((4R/S)-3,3-dimethyl-4-(1-pyrrolidinyl)-1-piperidinyl)-2-oxoethyl)-3,4-dihydro-2(1H)-pyrazinone;
(3R)-4-((4-chloro-2,5-dimethylphenyl)sulfonyl)-3-(2-((3R)-3-(dimethylamino)-1-piperidinyl)-2-oxoethyl)-3,4-dihydro-2(1H)-pyrazinone trifluoroacetic acid salt;
(3R)-4-((4-chloro-2,5-dimethylphenyl)sulfonyl)-3-(2-(7-methyl-2,7-diazaspiro-[4.4]non-2-yl)-2-oxoethyl)-3,4-dihydro-2(1H)-pyrazinone;
(3R)-4-((4-chloro-2,5-dimethylphenyl)sulfonyl)-3-(2-((4R)-4-(dimethylamino)-1-azocanyl)-2-oxoethyl)-3,4-dihydro-2(1H)-pyrazinone;
(3R)-4-((2,3-dichlorophenyl)sulfonyl)-3-(2-((4R/S)-4-(dimethylamino)-1-azocanyl)-2-oxoethyl)-3,4-dihydro-2(1H)-pyrazinone;
(3R)-3-(2-(2,7-diazaspiro[4.4]non-2-yl)-2-oxoethyl)-4-((2,3-dichlorophenyl)-sulfonyl)-3,4-dihydro-2(1H)-pyrazinone;
(3R)-4-((2,3-dichlorophenyl)sulfonyl)-3-(2-(7-(1-methylethyl)-2,7-diazaspiro[4.4]non-2-yl)-2-oxoethyl)-3,4-dihydro-2(1H)-pyrazinone;
(3R)-4-((4-chloro-2,5-dimethylphenyl)sulfonyl)-3-(2-(7-(1-methylethyl)-2,7-diazaspiro[4.4]non-2-yl)-2-oxoethyl)-3,4-dihydro-2(1H)-pyrazinone;
(3R)-4-((4-chloro-2,5-dimethylphenyl)sulfonyl)-3-(2-(7-cyclopropyl-2,7-diazaspiro[4.4]non-2-yl)-2-oxoethyl)-3,4-dihydro-2(1H)-pyrazinone;
(3R)-4-((2,3-dichlorophenyl)sulfonyl)-3-(2-(7-methyl-2,7-diazaspiro[4.4]non-2-yl)-2-oxoethyl)-3,4-dihydro-2(1H)-pyrazinone;
N-(1-(2-((2R)-1-((4-methylphenyl)sulfonyl)-3-oxo-1,2,3,4-tetrahydro-2-pyrazinyl)acetyl)-4-piperidinyl)benzamide;
(3R)-3-(2-(4-((1-methylethyl)amino)-1-piperidinyl)-2-oxoethyl)-4-((4-methylphenyl)sulfonyl)-3,4-dihydro-2(1H)-pyrazinone;
(3R)-4-((4-methylphenyl)sulfonyl)-3-(2-oxo-2-(2RS-(phenylmethyl)-1-pyrrolidinyl)ethyl)-3,4-dihydro-2(1H)-pyrazinone;
(3R)-4-((4-methylphenyl)sulfonyl)-3-(2-oxo-2-(2RS-phenyl-1-pyrrolidinyl)-ethyl)-3,4-dihydro-2(1H)-pyrazinone;
(3R)-4-((4-methylphenyl)sulfonyl)-3-(2-oxo-2-(4-(phenyloxy)-1-piperidinyl)-ethyl)-3,4-dihydro-2(1H)-pyrazinone;
(3R)-4-((4-methylphenyl)sulfonyl)-3-(2-oxo-2-(3-oxo-1-piperazinyl)ethyl)-3,4-dihydro-2(1H)-pyrazinone;
(3R)-3-(2-(2-RS-((methyloxy)methyl)-1-pyrrolidinyl)-2-oxoethyl)-4-((4-methyl-phenyl)sulfonyl)-3,4-dihydro-2(1H)-pyrazinone;
(3R)-3-(2-(2-RS-((4-(methyloxy)phenyl)methyl)-1-pyrrolidinyl)-2-oxoethyl)-4-((4-methylphenyl)sulfonyl)-3,4-dihydro-2(1H)-pyrazinone;
(3R)-3-(2-(4-(4-methylphenyl)-1-piperidinyl)-2-oxoethyl)-4-((4-methylphenyl)-sulfonyl)-3,4-dihydro-2(1H)-pyrazinone;
8-(((2R)-1-((4-methylphenyl)sulfonyl)-3-oxo-1,2,3,4-tetrahydro-2-pyrazinyl)-acetyl)-2,8-diazaspiro[4.5]decan-1-one;
(3R)-3-(2-(2-RS-(1H-indol-2-yl)-1-pyrrolidinyl)-2-oxoethyl)-4-((4-methyl-phenyl)sulfonyl)-3,4-dihydro-2(1H)-pyrazinone;
(3R)-3-(2-(4-((2-chlorophenyl)oxy)-1-piperidinyl)-2-oxoethyl)-4-((4-methylphenyl)sulfonyl)-3,4-dihydro-2(1H)-pyrazinone;
(3R)-4-((4-methylphenyl)sulfonyl)-3-(2-oxo-2-(2-RS-phenyl-4-thiomorpholinyl)ethyl)-3,4-dihydro-2(1H)-pyrazinone;
(3R)-4-((4-methylphenyl)sulfonyl)-3-(2-oxo-2-(2-RS-phenyl-4-morpholinyl)-ethyl)-3,4-dihydro-2(1H)-pyrazinone;
(3R)-3-(2-(4-(1H-indol-3-yl)-1-piperidinyl)-2-oxoethyl)-4-((4-methylphenyl)-sulfonyl)-3,4-dihydro-2(1H)-pyrazinone;
(3R)-3-(2-(2-(2-RS-methylphenyl)-1-pyrrolidinyl)-2-oxoethyl)-4-((4-methyl-phenyl)sulfonyl)-3,4-dihydro-2(1H)-pyrazinone;
(3R)-4-((2,3-dichlorophenyl)sulfonyl)-3-(2-(4-methyl-1,4-diazepan-1-yl)-2-oxoethyl)-3,4-dihydro-2(1H)-pyrazinone; and
(3R)-4-((2,3-dichlorophenyl)sulfonyl)-3-(2-((3R)-3-(dimethylamino)-1-pyrrolidinyl)-2-oxoethyl)-3,4-dihydro-2(1H)-pyrazinone; or
a pharmaceutically acceptable salt thereof.

24. (canceled)

25. (canceled)

26. The compound of claim 1 wherein R² is alkyl, haloalkyl, cycloalkyl, cycloalkylalkyl, hydroxyalkyl, alkoxyalkyl, or aminoalkyl.

27. (canceled)

28. (canceled)

29. (canceled)

30. The compound of claim 3 wherein R³ is a group of formula: 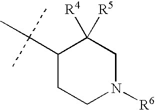
wherein R⁴ and R⁵ are independently alkyl.

31. The compound of claim 30 wherein R⁶ is hydrogen, alkyl, haloalkyl, alkoxycarbonyl, cycloalkyl, cycloalkylalkyl, aralkyl, heteroaralkyl, heteroaryl, aryl, acyl, aminoalkyl, or alkoxyalkyl.

32. (canceled)

33. (canceled)

34. (canceled)

35. (canceled)

36. (canceled)

37. The compound of claim 30 wherein R is hydrogen and R¹ is phenyl substituted with R^{a}, R^{b}, or R^{c} independently selected from hydrogen, alkyl, or halo.

38. The compound of claim 30 wherein R is hydrogen and R¹ is heteroaryl substituted with R^{a}, R^{b}, or R^{c} independently selected from hydrogen, alkyl, or halo.

39. The compound of claim 30 wherein R and R² are hydrogen, R¹ is 4-methylphenyl, 2,3-dichlorophenyl, 3,4-dichlorophenyl, and 2,5-dimethyl-4-chlorophenyl, and R³ is 3,3-dimethylpiperidin-4-yl, 1-methyl-3,3-dimethylpiperidin-4-yl, 1-(isobutyl)-3,3-dimethylpiperidin-4-yl, 1-(pyridin-2-ylmethyl)-3,3-dimethyl-piperidin-4-yl, 1-(2-propyl)-3,3-dimethylpiperidin-4-yl, 1-(pyridin-4-yl-methyl)-3,3-dimethylpiperidin-4-yl, 1-(acetyl)-3,3-dimethylpiperidin-4-yl, 1-(tert-butyoxy-carbonyl)-3,3-dimethyl-piperidin-4-yl, 1-cyclopropyl-3,3-dimethylpiperidin-4-yl, 1-(pyridin-3-ylmethyl)-3,3-dimethylpiperidin-4-yl, 1-(2-methoxyethyl)-3,3-dimethyl-piperidin-4-yl, 3,3-dimethyl-1-(2,2,2-trifluoroethyl)piperidin-4-yl, 1-(tert-butyoxy-carbonyl)piperidin-3-yl, 1-ethyl-3,3-dimethylpiperidin-4-yl, 1-n-propyl-3,3-dimethyl-piperidin-4-yl, 1-2,2-dimethylpropyl-3,3-dimethylpiperidin-4-yl, 1-cyclobutyl-3,3-dimethylpiperidin-4-yl, or 1-cyclopentyl-3,3-dimethylpiperidin-4-yl wherein the stereochemistry at the carbon atom at the 4-position of the piperidin-4-yl ring is (R), (S), or (RS).

40. (canceled)

41. (canceled)

42. (canceled)

43. A pharmaceutical composition comprising a compound of claim 1 and a pharmaceutically acceptable excipient.

44. A method of treating a disease in a patient mediated the B1 receptor comprising administering to the patient a pharmaceutical composition comprising a therapeutically effective amount of a compound claim 1 and a pharmaceutically acceptable excipient.
